SEQUENCE LISTING (165 sequences)

SEQ ID NO: 1 - protein, 11 aa, Artificial Sequence
  vMSP_001
MGARASVLSGG

SEQ ID NO: 2 - protein, 10 aa, Artificial Sequence
  vMSP_002
MGARASVLSG

SEQ ID NO: 3 - protein, 6 aa, Artificial Sequence
  vMSP_003
MGGKLS

SEQ ID NO: 4 - protein, 8 aa, Artificial Sequence
  vMSP_004
MGARASGG

SEQ ID NO: 5 - protein, 6 aa, Artificial Sequence
  vMSP_005
MGARAS

SEQ ID NO: 6 - protein, 9 aa, Artificial Sequence
  vMSP_006
MGARASVLS

SEQ ID NO: 7 - protein, 11 aa, Artificial Sequence
  vMSP_007
MGSKLTCCLGG

SEQ ID NO: 8 - protein, 11 aa, Artificial Sequence
  vMSP_008
MGNILTCCINS

SEQ ID NO: 9 - protein, 11 aa, Artificial Sequence
  vMSP_009
MGSCVSRDLFT

SEQ ID NO: 10 - protein, 10 aa, Artificial Sequence
  vMSP_0010
MGGNHSHKPP

SEQ ID NO: 11 - protein, 12 aa, Artificial Sequence
  vMSP_011
MGSCVSRDLFTS

SEQ ID NO: 12 - protein, 7 aa, Artificial Sequence
  vMSP_012
MGARASG

SEQ ID NO: 13 - protein, 27 aa, Artificial Sequence
  vED_001
KKKKKRFSFKKSFKLSGFSFKKNKKAS

SEQ ID NO: 14 - protein, 9 aa, Artificial Sequence
  vED_002
KKFSFKKFG

SEQ ID NO: 15 - protein, 11 aa, Artificial Sequence
  vED_003
KKFSFKKNKKG

SEQ ID NO: 16 - protein, 11 aa, Artificial Sequence
  vED_004
KKKKFSFKKFG

SEQ ID NO: 17 - protein, 11 aa, Artificial Sequence
  vED_005
KKKKFAFKKFG

SEQ ID NO: 18 - protein, 9 aa, Artificial Sequence
  vED_006
KKFAFKKFG

SEQ ID NO: 19 - protein, 27 aa, Artificial Sequence
  vED_007
KKKKKRFAFKKAFKLAGFAFKKNKKAG

SEQ ID NO: 20 - protein, 26 aa, Artificial Sequence
  vED_008
KKKKRFSFKKPFKLSGLSFKRNRKAS

SEQ ID NO: 21 - protein, 5 aa, Artificial Sequence
  vED_009
KKKKG

SEQ ID NO: 22 - protein, 27 aa, Artificial Sequence
  vED_010
KKKKKRFSFKKSFKLAGFSFKKNKKAS

SEQ ID NO: 23 - protein, 27 aa, Artificial Sequence
  vED_011
KKKKRFSFKKPFKLSGLSFKRNRKASG

SEQ ID NO: 24 - protein, 12 aa, Artificial Sequence
  vED_012
KKKKKFSFKKAS

SEQ ID NO: 25 - protein, 16 aa, Artificial Sequence
  vED_013
KKKKKFLSFKRNRKAS

SEQ ID NO: 26 - protein, 31 aa, Artificial Sequence
  vED_014
KKKKRFSFKKPFKLSGLSFKRNRKASGGGGG

SEQ ID NO: 27 - protein, 26 aa, Artificial Sequence
  vED_015
KKKKKFSFKKPFKLSGLSFKRNRKAS

SEQ ID NO: 28 - protein, 27 aa, Artificial Sequence
  vED_016
KKKKKRFSFKKPFKLSGLSFKRNRKAS

SEQ ID NO: 29 - protein, 12 aa, Artificial Sequence
  vED_017
KKKKRFSFKKAS

SEQ ID NO: 30 - protein, 24 aa, Artificial Sequence
  vED_018
KKKKKFSFKKGGGGSGGGGSGAGL

SEQ ID NO: 31 - protein, 19 aa, Artificial Sequence
  vMYRED_001
MGARASVLSGKKFSFKKFG

SEQ ID NO: 32 - protein, 21 aa, Artificial Sequence
  vMYRED_002
MGARASVLSGKKFSFKKNKKG

SEQ ID NO: 33 - protein, 21 aa, Artificial Sequence
  vMYRED_003
MGARASVLSGKKKKFSFKKFG

SEQ ID NO: 34 - protein, 21 aa, Artificial Sequence
  vMYRED_004
MGARASVLSGKKKKFAFKKFG

SEQ ID NO: 35 - protein, 19 aa, Artificial Sequence
  vMYRED_005
MGARASVLSGKKFAFKKFG

SEQ ID NO: 36 - protein, 38 aa, Artificial Sequence
  vMYRED_006
MGARASVLSGGKKKKKRFAFKKAFKLAGFAFKKNKKAG

SEQ ID NO: 37 - protein, 38 aa, Artificial Sequence
  vMYRED_007
MGARASVLSGGKKKKRFSFKKPFKLSGLSFKRNRKASG

SEQ ID NO: 38 - protein, 11 aa, Artificial Sequence
  vMYRED_008
MGGKLSKKKKG

SEQ ID NO: 39 - protein, 39 aa, Artificial Sequence
  vMYRED_009
MGARASVLSGGKKKKKRFSFKKSFKLAGFSFKKNKKASG

SEQ ID NO: 40 - protein, 35 aa, Artificial Sequence
  vMYRED_010
MGARASGGKKKKRFSFKKPFKLSGLSFKRNRKASG

SEQ ID NO: 41 - protein, 33 aa, Artificial Sequence
  vMYRED_011
MGARASKKKKRFSFKKPFKLSGLSFKRNRKASG

SEQ ID NO: 42 - protein, 36 aa, Artificial Sequence
  vMYRED_012
MGARASVLSKKKKRFSFKKPFKLSGLSFKRNRKASG

SEQ ID NO: 43 - protein, 24 aa, Artificial Sequence
  vMYRED_013
MGARASVLSGGKKKKKFSFKKASG

SEQ ID NO: 44 - protein, 28 aa, Artificial Sequence
  vMYRED_014
MGARASVLSGGKKKKKFLSFKRNRKASG

SEQ ID NO: 45 - protein, 38 aa, Artificial Sequence
  vMYRED_015
MGSKLTCCLGGKKKKRFSFKKPFKLSGLSFKRNRKASG

SEQ ID NO: 46 - protein, 39 aa, Artificial Sequence
  vMYRED_016
MGNILTCCINSGKKKKRFSFKKPFKLSGLSFKRNRKASG

SEQ ID NO: 47 - protein, 40 aa, Artificial Sequence
  vMYRED_017
MGSCVSRDLFTSGKKKKRFSFKKPFKLSGLSFKRNRKASG

SEQ ID NO: 48 - protein, 39 aa, Artificial Sequence
  vMYRED_018
MGGNHSHKPPGGKKKKRFSFKKPFKLSGLSFKRNRKASG

SEQ ID NO: 49 - protein, 38 aa, Artificial Sequence
  vMYRED_019
MGARASVLSGGKKKKKFSFKKPFKLSGLSFKRNRKASG

SEQ ID NO: 50 - protein, 51 aa, Artificial Sequence
  vMYRED_020
MGARASVLSGGGGSGGGGSKKKKKRFSFKKPFKLSGLSFKRNRKASGGGGG

SEQ ID NO: 51 - protein, 48 aa, Artificial Sequence
  vMYRED_021
MGARASGGGGSGGGGSKKKKKRFSFKKPFKLSGLSFKRNRKASGGGGG

SEQ ID NO: 52 - protein, 55 aa, Artificial Sequence
  vMYRED_022
MGARASVLSGGGGSGGGGSGGGGKKKKKRFSFKKPFKLSGLSFKRNRKASGGGGG

SEQ ID NO: 53 - protein, 53 aa, Artificial Sequence
  vMYRED_023
MGARASGGGGSGGGGSGGGGSKKKKKRFSFKKPFKLSGLSFKRNRKASGGGGG

SEQ ID NO: 54 - protein, 39 aa, Artificial Sequence
  vMYRED_024
MGARASVLSGGKKKKKRFSFKKSFKLSGFSFKKNKKASG

SEQ ID NO: 55 - protein, 69 aa, Artificial Sequence
  vMYRED_025
MGARASVLSGGGGSGAGLLKMFNKATDAVSKMGGGGSKKKKKRFSFKKSFKLSGFSFKKNKKASGGGGG

SEQ ID NO: 56 - protein, 38 aa, Artificial Sequence
  vMYRED_026
MGARASVLSGGGGSGAGLLKMFNKATDAVSKMGGGGSG

SEQ ID NO: 57 - protein, 27 aa, Artificial Sequence
  vMYRED_027
MGSCVSRDLFTSGGKKKKKFSFKKASG

SEQ ID NO: 58 - protein, 21 aa, Artificial Sequence
  vMYRED_028
MGARASGGKKKKKFSFKKASG

SEQ ID NO: 59 - protein, 19 aa, Artificial Sequence
  vMYRED_029
MGARASKKKKKFSFKKASG

SEQ ID NO: 60 - protein, 21 aa, Artificial Sequence
  vMYRED_030
MGARASGGKKKKRFSFKKASG

SEQ ID NO: 61 - protein, 17 aa, Artificial Sequence
  vMYRED_031
MGSCVSRDLFTSGGASG

SEQ ID NO: 62 - protein, 35 aa, Artificial Sequence
  vMYRED_032
MGARASGGGGSGGGGSGGGGSGKKKKKFSFKKASG

SEQ ID NO: 63 - protein, 45 aa, Artificial Sequence
  vMYRED_033
MGARASGKKKKKFSFKKGGGGSGGGGSGAGLLKMFNKATDAVSKM

SEQ ID NO: 64 - protein, 50 aa, Artificial Sequence
  vMYRED_034
MGARASGGGGSGKKKKKFSFKKGGGGSGGGGSGAGLLKMFNKATDAVSKM

SEQ ID NO: 65 - protein, 41 aa, Artificial Sequence
  vMYRED_035
MGARASVLSGGKKKKKFSFKKASGGGGRHRQPRGWEQLKSG

SEQ ID NO: 66 - protein, 37 aa, Artificial Sequence
  vMYRED_036
MGARASVLSGGKKKKKFSFKKASGGGGPRRARSVKSG

SEQ ID NO: 67 - protein, 37 aa, Artificial Sequence
  vMYRED_037
MGARASVLSGGKKKKKFSFKKASGGGGSRRKREVKSG

SEQ ID NO: 68 - protein, 35 aa, Artificial Sequence
  vMYRED_038
MGARASVLSGGKKKKKFSFKKASGGGKSRREVESG

SEQ ID NO: 69 - protein, 35 aa, Artificial Sequence
  vMYRED_039
MGARASVLSGGKKKKKFSFKKASGGGKKRKKRESG

SEQ ID NO: 70 - protein, 33 aa, Artificial Sequence
  vMYRED_040
MGARASVLSGGKKKKKFSFKKASGGGRARRESG

SEQ ID NO: 71 - protein, 28 aa, Artificial Sequence
  vMYRED_041
MGARASVLSGGKKKKKFSFKKRARRESG

SEQ ID NO: 72 - protein, 28 aa, Artificial Sequence
  vMYRED_042
MGARASVLSGGKKKKKFSFKKRKKRESG

SEQ ID NO: 73 - protein, 17 aa, Artificial Sequence
  vMYRED_043
MGSRVSRDLFTSGGASG

SEQ ID NO: 74 - protein, 13 aa, Artificial Sequence
  vMYRED_044
MGSAVSRDLFTSG

SEQ ID NO: 75 - protein, 44 aa, Artificial Sequence
  vMYRED_045
MGSCVSRDLFTSGGKKKKKFSFKKASGGGGRHRQPRGWEQLKSG

SEQ ID NO: 76 - protein, 40 aa, Artificial Sequence
  vMYRED_046
MGSCVSRDLFTSGGKKKKKFSFKKASGGGGPRRARSVKSG

SEQ ID NO: 77 - protein, 40 aa, Artificial Sequence
  vMYRED_047
MGSCVSRDLFTSGGKKKKKFSFKKASGGGGSRRKREVKSG

SEQ ID NO: 78 - protein, 38 aa, Artificial Sequence
  vMYRED_048
MGSCVSRDLFTSGGKKKKKFSFKKASGGGKSRREVESG

SEQ ID NO: 79 - protein, 38 aa, Artificial Sequence
  vMYRED_049
MGSCVSRDLFTSGGKKKKKFSFKKASGGGKKRKKRESG

SEQ ID NO: 80 - protein, 36 aa, Artificial Sequence
  vMYRED_050
MGSCVSRDLFTSGGKKKKKFSFKKASGGGRARRESG

SEQ ID NO: 81 - protein, 31 aa, Artificial Sequence
  vMYRED_051
MGSCVSRDLFTSGGKKKKKFSFKKRARRESG

SEQ ID NO: 82 - protein, 31 aa, Artificial Sequence
  vMYRED_052
MGSCVSRDLFTSGGKKKKKFSFKKRKKRESG

SEQ ID NO: 83 - protein, 36 aa, Artificial Sequence; identical to SEQ ID NO: 80
  vMYRED_053

SEQ ID NO: 84 - protein, 48 aa, Artificial Sequence
  vMYRED_054
MGSCVSRDLFTSGGKKKKKFSFKKASGGGSGGGGSEDQVDPRLIDGKG

SEQ ID NO: 85 - protein, 48 aa, Artificial Sequence
  vMYRED_055
MGSRVSRDLFTSGGKKKKKFSFKKASGGGSGGGGSEDQVDPRLIDGKG

SEQ ID NO: 86 - protein, 48 aa, Artificial Sequence
  vMYRED_056
MGSCVSRDLFTSGGKRKRKFSFKRASGGGSGGGGSEDQVDPRLIDGKG

SEQ ID NO: 87 - protein, 47 aa, Artificial Sequence
  vMYRED_057
MGGLFSRWRTSGGKKKKKFSFKKASGGGSGGGGSEDQVDPRLIDGKG

SEQ ID NO: 88 - protein, 47 aa, Artificial Sequence
  vMYRED_058
MGQNLSTSNPLGGKKKKKFSFKKASGGGSGGGGSEDQVDPRLIDGKG

SEQ ID NO: 89 - protein, 54 aa, Artificial Sequence
  vMYRED_059
MKLSLVAAMLLLLSAARAGGKKKKKFSFKKASGGGSGGGGSEDQVDPRLIDGKG

SEQ ID NO: 90 - protein, 65 aa, Artificial Sequence
  vMYRED_060
MKLSLVAAMLLLLSAARAGSCVSRDLFTSGGKKKKKFSFKKASGGGSGGGGSEDQVDPRLIDGKG

SEQ ID NO: 91 - protein, 66 aa, Artificial Sequence
  vMYRED_061
MGLQACLLGLFALILSGKCGSCVSRDLFTSGGKKKKKFSFKKASGGGSGGGGSEDQVDPRLIDGKG

SEQ ID NO: 92 - protein, 63 aa, Artificial Sequence
  vMYRED_062
MGVRHPLCSRRLLAVCALVSLATAALLGGKKKKKFSFKKASGGGSGGGGSEDQVDPRLIDGKG

SEQ ID NO: 93 - protein, 51 aa, Artificial Sequence
  vMYRED_063
MGKSESQMDITDINTGGKKKKKFSFKKASGGGSGGGGSEDQVDPRLIDGKG

SEQ ID NO: 94 - protein, 57 aa, Artificial Sequence
  vMYRED_064
MGNEASLEGGAGEGPLPPGGSGGKKKKKFSFKKASGGGSGGGGSEDQVDPRLIDGKG

SEQ ID NO: 95 - protein, 63 aa, Artificial Sequence
  vMYRED_065
MGQQPAKSMDVRRIEGGELLLNQLAGRGGKKKKKFSFKKASGGGSGGGGSEDQVDPRLIDGKG

SEQ ID NO: 96 - protein, 94 aa, Artificial Sequence
  vMYRED_066
MGQIVTFFQEVPHVIEEVMNIVLIALSVLAVLKGLYNFATCGLVGLVTFLLLCGRSCTGGKKKKKFSFKKASGGGSGGGGSEDQVDPRLIDGKG

SEQ ID NO: 97 - protein, 94 aa, Artificial Sequence
  vMYRED_067
MGQLISFFQEIPVFLQEALNIALVAVSLIAVIKGIINLYKSGLFQFIFFLLLAGRSCSGGKKKKKFSFKKASGGGSGGGGSEDQVDPRLIDGKG

SEQ ID NO: 98 - protein, 39 aa, Artificial Sequence
  vMYRED_068
MGSCVSRDLFTSGGKKKKKFSFKKASGGGGRRARSVKSG

SEQ ID NO: 99 - protein, 39 aa, Artificial Sequence
  vMYRED_069
MGSCVSRDLFTSGGKKKKKFSFKKASGGGGPRRARSVSG

SEQ ID NO: 100 - protein, 52 aa, Artificial Sequence
  vMYRED_070
MGSCVSRDLFTSGGKKKKKFSFKKASGGGGPRRARSVKSEDQVDPRLIDGKG

SEQ ID NO: 101 - protein, 48 aa, Artificial Sequence
  vMYRED_071
MGNEASLEGGAGGGKKKKKFSFKKASGGGSGGGGSEDQVDPRLIDGKG

SEQ ID NO: 102 - protein, 62 aa, Artificial Sequence
  vMYRED_072
MGNEVSLEGGAGDGPLPPGGAGPGPGPGPGSHPQLPAAKKRKSLASRKPPVEDPAAEDLRTR

SEQ ID NO: 103 - protein, 66 aa, Artificial Sequence
  vMYRED_073
MGNEASLEGGAGEGPLPPGGSGGKKKKKFSFKKASGGGGPRRARSVKSGGGGSEDQVDPRLIDGKG

SEQ ID NO: 104 - protein, 46 aa, Artificial Sequence
  vMYRED_074
MGQQPAKSMDGGKKKKKFSFKKASGGGSGGGGSEDQVDPRLIDGKG

SEQ ID NO: 105 - protein, 35 aa, Artificial Sequence
  vMYRED_075
MKKKKKFSFKKASGGGSGGGGSEDQVDPRLIDGKG

SEQ ID NO: 106 - protein, 91 aa, Artificial Sequence
  vMYRED_076
MGCKWSKSSVIGWPAVRERMRRAEPAADGVGAVSRDLEKHGAITSSNTAANNAACAWLEAQEEEEVGFPGGGSGGGGSEDQVDPRLIDGKG

SEQ ID NO: 107 - protein, 107 aa, Artificial Sequence
  vMYRED_077
MGCKWSKSSVIGWPAVRERMRRAEPAADGVGAVSRDLEKHGAITSSNTAANNAACAWLEAQEEEEVGFPGGGSKKKKKFSFKKASGGGSGGGGSEDQVDPRLIDGKG

SEQ ID NO: 108 - protein, 33 aa, Artificial Sequence
  vMYRED_078
ASGGGGSEDQVDPRLIDGKSGGGKKKKKFSFKK

SEQ ID NO: 109 - protein, 64 aa, Artificial Sequence
  vMYRED_079
MKLSLVAAMLLLLSAARAGARASVLSGGGGKKKKKFSFKKASGGGSGGGGSEDQVDPRLIDGKG

SEQ ID NO: 110 - protein, 76 aa, Artificial Sequence
  vMYRED_080
MKLSLVAAMLLLLSAARAGNEASLEGGAGEGPLPPGGSGGGGKKKKKFSFKKASGGGSGGGGSEDQVDPRLIDGKG

SEQ ID NO: 111 - protein, 61 aa, Artificial Sequence
  vMYRED_081
ASGGGGSEDQVDPRLIDGKSGGGKKKKKFSFKKGGGGSGGGGAITCLSFITFYVAAFMVLL

SEQ ID NO: 112 - protein, 47 aa, Artificial Sequence
  vMYRED_082
ASGGGGSEDQVDPRLIDGKSGGGSGGGGAITCLSFITFYVAAFMVLL

SEQ ID NO: 113 - protein, 73 aa, Artificial Sequence
  vMYRED_083
ASGGGGSEDQVDPRLIDGKSGGGKKKKKFSFKKGGGGSGGGGARIKFSTGSHCHGSFSLIFLSLWAVIFVLYQ

SEQ ID NO: 114 - protein, 78 aa, Artificial Sequence
  vMYRED_084
ASGGGGSEDQVDPRLIDGKSGGGKKKKKFSFKKGGGGSGGGGKLLNATACDGELSSSGTSSSKGIIFYVLFSILYLIF

SEQ ID NO: 115 - protein, 66 aa, Artificial Sequence
  vMYRED_085
ASGGGGSEDQVDPRLIDGKSGGGKKKKKFSFKKGGGGSGGGGRRNVARYGRTVVVSGSHAAAVAPG

SEQ ID NO: 116 - protein, 82 aa, Artificial Sequence
  vMYRED_086
ASGGGGSEDQVDPRLIDGKSGGGKKKKKFSFKKGGGGSGGGGLGQLEASCRTNYGYSAAPSLHLPPGSLLASLVPLLLLSLP

SEQ ID NO: 117 - protein, 74 aa, Artificial Sequence
  vMYRED_087
ASGGGGSEDQVDPRLIDGKSGGGKKKKKFSFKKGGGGSGGGGRTNYGYSAAPSLHLPPGSLLASLVPLLLLSLP

SEQ ID NO: 118 - protein, 47 aa, Artificial Sequence
  vMYRED_088
ASGGGGSEDQVDPRLIDGKSGGGKHKEKMSKDGKKKKKKSKTKCVIM

SEQ ID NO: 119 - protein, 51 aa, Artificial Sequence
  vMYRED_089
ASGGGGSEDQVDPRLIDGKSGGGKHKEKMSKDGKKKKKFSFKKSKTKCVIM

SEQ ID NO: 120 - protein, 41 aa, Artificial Sequence
  vMYRED_090
ASGGGGSEDQVDPRLIDGKSGGGKKKKKFSFKKSKTKCVIM

SEQ ID NO: 121 - protein, 37 aa, Artificial Sequence
  vMYRED_091
ASGGGGSEDQVDPRLIDGKSGGGKKKKKFSFKKCVIM

SEQ ID NO: 122 - protein, 38 aa, Artificial Sequence
  vMYRED_092
ASGGGGSEDQVDPRLIDGKSGGGKKKKKFSFKKRCVIM

SEQ ID NO: 123 - protein, 46 aa, Artificial Sequence
  vMYRED_093
ASGGGGSEDQVDPRLIDGKSGGGKKKKKFSFKKGGGSGGGGSCVIM

SEQ ID NO: 124 - protein, 46 aa, Artificial Sequence
  vMYRED_094
ASGGGGSEDQVDPRLIDGKSGGGKKKKKFSFKKGGGSGGGGSCCLL

SEQ ID NO: 125 - protein, 59 aa, Artificial Sequence
  vMYRED_095
MGNEASLEGGAGEGPLPPGGSGGKKKKKFSFKKASGGGKSRREVESGGGGDYKDDDDKG

SEQ ID NO: 126 - protein, 48 aa, Artificial Sequence
  vMYRED_096
MGNEASLEGGGGKKKKKFSFKKASGGGKSRREVESGGGGDYKDDDDKG

SEQ ID NO: 127 - protein, 28 aa, Artificial Sequence
  vMYRED_097
MGNEASLEGGGGKKKKKFSFKKASGGGG

SEQ ID NO: 128 - protein, 65 aa, Artificial Sequence
  vMYRED_098
MGQQPAKSMDVRRIEGGELLLNQLAGRGGKKKKKFSFKKASGGGKSRREVESGGGGDYKDDDDKG

SEQ ID NO: 129 - protein, 54 aa, Artificial Sequence
  vMYRED_099
MGQQPAKSMDVRRIEGGGKKKKKFSFKKASGGGKSRREVESGGGGDYKDDDDKG

SEQ ID NO: 130 - protein, 34 aa, Artificial Sequence
  vMYRED_100
MGQQPAKSMDVRRIEGGGKKKKKFSFKKASGGGG

SEQ ID NO: 131 - protein, 50 aa, Artificial Sequence
  vMYRED_101
MGNEASLEGGAGEGPLPPGGSGGKKKKKFSFKKASGGGKSRREVESGGGG

SEQ ID NO: 132 - protein, 49 aa, Artificial Sequence
  vMYRED_27b
MGSCVSRDLFTSGGKKKKKFSFKKASPRRARSVKSSGGGGDYKDDDDKG

SEQ ID NO: 133 - protein, 50 aa, Artificial Sequence
  vMYRED_27c
MGSCVSRDLFTSGGKKKKKFSFKKASGGGKSRREVESGGGGDYKDDDDKA

SEQ ID NO: 134 - protein, 62 aa, Artificial Sequence
  vMYRED_64b
MGNEASLEGGAGEGPLPPGGSGGKKKKKFSFKKASGGGGPRRARSVKSSGGGGDYKDDDDKA

SEQ ID NO: 135 - protein, 59 aa, Artificial Sequence
  vMYRED_64c
MGNEASLEGGAGEGPLPPGGSGGKKKKKFSFKKASGGGKSRREVESGGGGDYKDDDDKA

SEQ ID NO: 136 - protein, 46 aa, Artificial Sequence
  vMYRED_64d
MGNEASLEGGAGEGPLPPGGSGGKKKKKFSFKKASGGGKSRREVEA

SEQ ID NO: 137 - protein, 65 aa, Artificial Sequence
  vMYRED_65c
MGQQPAKSMDVRRIEGGELLLNQLAGRGGKKKKKFSFKKASGGGKSRREVESGGGGDYKDDDDKA

SEQ ID NO: 138 - protein, 52 aa, Artificial Sequence
  vMYRED_65d
MGQQPAKSMDVRRIEGGELLLNQLAGRGGKKKKKFSFKKASGGGKSRREVEA

SEQ ID NO: 139 - protein, 70 aa, Artificial Sequence
  vMYRED_81b
ASGGGGSDYKDDDDKRPRAVPTQSGGGSGGGGKKKKKFSFKKGGGGSGGGGAITCLSFITFYVAAFMVLL

SEQ ID NO: 140 - protein, 56 aa, Artificial Sequence
  vMYRED_88b
ASGGGGSDYKDDDDKRPRAVPTQSGGGSGGGGKHKEKMSKDGKKKKKKSKTKCVIM

SEQ ID NO: 141 - protein, 60 aa, Artificial Sequence
  vMYRED_89b
ASGGGGSDYKDDDDKRPRAVPTQSGGGSGGGGKHKEKMSKDGKKKKKFSFKKSKTKCVIM

SEQ ID NO: 142 - protein, 54 aa, Artificial Sequence
  vMYRED_93b
AGGGGSDYKDDDDKRPRAVPTQSGGGSGGGGKKKKKFSFKKGGGSGGGGSCVIM

SEQ ID NO: 143 - protein, 1 aa, Artificial Sequence
  vLINKER_01
G

SEQ ID NO: 144 - protein, 2 aa, Artificial Sequence
  vLINKER_02
SG

SEQ ID NO: 145 - protein, 2 aa, Artificial Sequence
  vLINKER_03
GG

SEQ ID NO: 146 - protein, 8 aa, Artificial Sequence
  vLINKER_04
GGSGGGGS

SEQ ID NO: 147 - protein, 12 aa, Artificial Sequence
  vLINKER_05
GGSGGGGSGGGG

SEQ ID NO: 148 - protein, 13 aa, Artificial Sequence
  vLINKER_06
GGSGGGGSGGGGS

SEQ ID NO: 149 - protein, 26 aa, Artificial Sequence
  vLINKER_07
GGSGAGLLKMFNKATDAVSKMGGGGS

SEQ ID NO: 150 - protein, 27 aa, Artificial Sequence
  vLINKER_08
GGSGAGLLKMFNKATDAVSKMGGGGSG

SEQ ID NO: 151 - protein, 3 aa, Artificial Sequence
  vLINKER_09
SSG

SEQ ID NO: 152 - protein, 14 aa, Artificial Sequence
  vLINKER_10
GGSGGGGSGGGGSG

SEQ ID NO: 153 - protein, 4 aa, Artificial Sequence
  vLINKER_11
GGSG

SEQ ID NO: 154 - protein, 15 aa, Artificial Sequence
  vSNX1_01
LLKMFNKATDAVSKM

SEQ ID NO: 155 - protein, 18 aa, Artificial Sequence
  vBIP_01
MKLSLVAAMLLLLSAARA

SEQ ID NO: 156 - protein, 28 aa, Artificial Sequence
  MBIP
MKLSLVAAMLLLLSLVAAMLLLLSAARA

SEQ ID NO: 157 - protein, 25 aa, Artificial Sequence
  MBIP2
MKLSLVAAMLLLLWVALLLLSAARA

SEQ ID NO: 158 - protein, 26 aa, Artificial Sequence
  MBIP3
MKLSLVAAMLLLLSLVALLLLSAARA

SEQ ID NO: 159 - protein, 26 aa, Artificial Sequence
  MBIP4
MKLSLVAAMLLLLALVALLLLSAARA

SEQ ID NO: 160 - protein, 276 aa, Artificial Sequence
  vMyrED27-mCherry
MGSCVSRDLFTSGGKKKKKFSFKKASGGGGGSGGGGGSAGSVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 161 - protein, 326 aa, Artificial Sequence
  vMyrED81-mCherry
MKLSLVAAMLLLLSAARAVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYKGGGGSGGGGGSAASGGGGSEDQVDPRLIDGKSGGGKKKKKFSFKKGGGGSGGGGAITCLSFITFYVAAFMVLL

SEQ ID NO: 162 - protein, 312 aa, Artificial Sequence
  vMyrED88-mCherry
MKLSLVAAMLLLLSAARAVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYKGGGGSGGGGGSAASGGGGSEDQVDPRLIDGKSGGGKHKEKMSKDGKKKKKKSKTKCVIM

SEQ ID NO: 163 - protein, 306 aa, Artificial Sequence
  vMyrEd64-mCherry
MGNEASLEGGAGEGPLPPGGSGGKKKKKFSFKKASGGGSGGGGSEDQVDPRLIDGKGGGGGSGGGGGSAGSVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 164 - protein, 316 aa, Artificial Sequence
  vMyrED89-mcherry
MKLSLVAAMLLLLSAARAVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYKGGGGSGGGGGSAASGGGGSEDQVDPRLIDGKSGGGKHKEKMSKDGKKKKKFSFKKSKTKCVIM

SEQ ID NO: 165 - protein, 4 aa, Artificial Sequence
  KRAS4B
CVIM

(19) United States
(12) Patent Application Publication
(10) Pub. No.: US 2024/0043494 A1
(43) Pub. Date: Feb. 8, 2024

(54) Vesicle Targeting Proteins And Uses Of Same

(71) Applicant: Amicus Therapeutics, Inc., Philadelphia, PA (US)

(72) Inventors: Joseph Bauman, Philadelphia, PA (US); Xiaoyang Wu, Edison, NJ (US); Yuliya McAnany, Philadelphia, PA (US)

(73) Assignee: Amicus Therapeutics, Inc., Philadelphia, PA (US)

(21) Appl. No.: 18/040,866

(22) PCT Filed: Aug. 6, 2021
(86) PCT No.: PCT/US21/45012

§ 371 Date: Feb. 7, 2023

Related U.S. Application Data
(60) Provisional application No. 63/160,215, filed on Mar. 12, 2021.
(60) Provisional application No. 63/062,844, filed on Aug. 7, 2020.

(51) Int. Cl. C07K 14/705; C07K 14/47

(52) U.S. Cl. CPC C07K 14/705; C07K 14/47; C07K 2319/02; C07K 2319/03

(57) ABSTRACT

The disclosure provides a novel method for treating genetic disorders where a peptide sequence targets proteins produced via gene therapy into exosomes. These protein-loaded exosomes can enter into non-transduced cells and correct pathology. Also, gene therapy compositions, protein replacement therapy composition, pharmaceutical compositions, methods of treatment, and uses of the gene therapy compositions and the recombinant proteins are also disclosed. The method can also be used to improve in vitro recombinant protein yield.

### TECHNICAL FIELD

The present disclosure generally relates to gene therapy and treating genetic disorders.

### BACKGROUND

Nucleic acids are routinely used in gene therapy for the replacement of non-functional genes and for neutralization of disease-causing mutations, and can be in the form of RNA, including interference (RNAi) effector molecules, and DNA. Despite this utility, naked DNA and RNA are known to be difficult to deliver in vivo due to rapid clearance, nucleases, lack of organ-specific distribution and low efficacy of cellular uptake; therefore, specialized gene delivery vehicles are usually used for delivery. Viral vectors and cationic liposomes have also been developed as delivery vehicles.

Despite these existing technologies, there remain significant limitations that restrict gene therapy, including immune recognition for most viral vectors and mutagenic integration for viruses such as lentiviruses; and inflammatory toxicity and rapid clearance for liposomes. Recognition by the innate immune system leads to acute inflammatory responses, which may require the use of immunosuppression strategies to overcome uptake and re-administration issues of current strategies potentially exposing patients to unwarranted risks of opportunistic infections. Antibodies generated against the delivery vehicles also dramatically decrease transgene expression on subsequent administration.

In particular, adeno-associated virus (AAV) vectors are one type that has been used to deliver transgenes for gene therapy. AAV is a helper-dependent virus of the family parvoviridae, subfamily parvovirinae, genus dependovirus, species adeno-associated virus. It requires a helper virus for replication, so natural infections take place in the context of infection with a helper virus such as adenovirus. AAV vectors are scalable, efficient, non-cytopathic gene delivery vehicles used primarily for the treatment of genetic diseases. Several AAV-based gene transfer products are now in clinical testing for a number of indications, and in some cases clinical development is approaching late-phase and licensing, including the US FDA approval of voretigene neparvovec (Luxturna), a novel gene therapy for the treatment of Leber's congenital amaurosis. Despite the promise of AAV based gene therapy approaches for treatment of a variety of disorders, immune responses occur following exposure to adeno-associated virus or AAV vectors, and these protective responses may limit therapeutic efficacy of AAV vectors. Humoral responses (anti-AAV neutralizing antibodies, or NAb) often give rise to viral neutralization causing a significant reduction in viral transduction of the target cell, thereby limiting the amount of therapeutic polypeptide delivered. Anti-AAV neutralizing antibodies can efficiently neutralize AAV vectors; this has been reported in humans (Manno C. S., et al., Nat Med. 2006; 12:342-347), mice (Scallan C. D., et al., Blood. 2006; 107:1810-1817), and non-human primates (Jiang H., et al., Blood. 2006; 108:3321-3328). Humans are also naturally exposed to wild type AAV, thus they develop antibodies that cross react with all AAV serotypes.

In addition to antibody responses, individuals exposed to AAV vectors can develop cytotoxic T cell (CTL) responses directed specifically against the AAV vector capsid (Li H., et al., Nature. 475, 217-221 (2011); Arruda V. R., et al., Blood. 115, 4678-4688 (2010); Chen Y. H., et al., Nat. Med. 15, 1215-1218 (2009); Boutin S., et al., Hum. Gene. Ther. 21, 704-712 (2010); Calcedo R., et al., J. Infect. Dis. 199, 381-390 (2009)). These CTL responses are responsible for the clearance of AAV vector transduced cells with consequent limited therapeutic efficacy. CTL responses are triggered in a vector dose-dependent manner (Li H., et al., Nature. 475, 217-221 (2011); Chen Y. H., et al., Nat. Med. 15, 1215-1218 (2009); Calcedo R., et al., J. Infect. Dis. 199, 381-390 (2009)), thus underlying the importance of achieving high levels of transduction efficiency at lower vector doses.

Current ways of solving immune response related problem involve pre-screening individuals and excluding those who have a high titer of neutralizing antibodies against the viral capsid. By lowering the necessary viral load for administration, there is less of a chance of an immune response to the capsid.

Another challenge associated with gene therapy is the incomplete correction of target tissue. 100% viral transduction (at least one vector copy per diseased cell-of-interest) is not possible with current methods. Usually, when transducing with genes coding for non-secreted proteins, the disease is ameliorated only in the few cells able to receive and stably express the AAV-delivered gene. This leads to only partial correction in a mosaic pattern. Ideally, the entire diseased tissue would be able to benefit from the transgene product.

Currently, one of the only options to maximize transduction is to increase viral dosage to maximize the chance of a viral vector entering the cell. This is problematic because higher dosage leads to a greater instance of adverse events, including serious adverse events.

Another option to improve transduction efficiency is to choose a more direct route of administration. For central nervous system (CNS) diseases, gene therapy vectors are injected into the cerebrospinal fluid (CSF) or directly into the brain in various routes, such as the intrathecal (IT), intracranial (IC), or intra cisterna magna (ICM) routes. These routes of administration are in contrast to intravenous (IV) delivery. Intravenous delivery is more routine and safer than delivery into the CSF and does not require specialized medical staff to perform. However, viral vectors including AAVs do not easily cross the blood brain barrier (BBB), and instead more easily transduce the liver and other non-CNS tissue.

While gene delivery technologies have improved, there remains issues including some cells (e.g. muscle satellite cells/stem cells) that may not be transduced (Arnett et al 2014 Mol Therapy—Methods & Clin Dev 1:14038). Additionally, effective transduction can be negatively affected by disease in some circumstances, e.g. muscle degeneration and fibrosis. However, with the increasing number of diseases shown to possess a genetic component, including obesity, heart disease and psychiatric illnesses, there is tremendous potential for the modification of susceptible genes for preemptive genetic solutions, but only if the risks are further reduced and long-term sustainability is achieved. Hence, it is imperative to develop technologies that are able to avoid immune recognition and inflammation, while retaining good delivery efficiencies, in order to expand the use of gene therapy beyond lethal diseases.

### SUMMARY

One aspect of the present disclosure pertains to a membrane associated-ED protein comprising one or more of a signal domain and an effector domain (ED). In some embodiments, the signal domain is directly connected to the effector domain. In some embodiments, the signal domain is connected to the effector domain by at least a first linker peptide. In some embodiments, the signal domain is a myristoylation signal domain.

In one or more embodiments of the membrane associated-ED protein, the signal domain amino acid sequence comprises at least 70% sequence similarity to SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 155, SEQ ID NO: 156, SEQ ID NO: 157, SEQ ID NO: 158, SEQ ID NO: 159 or SEQ ID NO: 165. In one or more embodiments, the resulting amino acid sequence of the membrane associated-ED protein is not SEQ ID NO: 38.

In one or more embodiments of the membrane associated-ED protein, the signal domain amino acid sequence comprises at least 90% sequence similarity to SEQ ID NO:1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 155, SEQ ID NO: 156, SEQ ID NO: 157, SEQ ID NO: 158, SEQ ID NO: 159 or SEQ ID NO: 165. In one or more embodiments, the resulting amino acid sequence of the membrane associated-ED protein is not SEQ ID NO: 38.

In one or more embodiments of the membrane associated-ED protein, the signal domain amino acid sequence comprises SEQ ID NO:1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 155, SEQ ID NO: 156, SEQ ID NO: 157, SEQ ID NO: 158, SEQ ID NO: 159 or SEQ ID NO: 165. In one or more embodiments, the resulting amino acid sequence of the membrane associated-ED protein is not SEQ ID NO: 38.

In one or more embodiments of the membrane associated-ED protein, the ED domain amino acid sequence comprises at least 70% sequence similarity to SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, or SEQ ID NO: 30 In one or more embodiments, the resulting amino acid sequence of the membrane associated-ED protein is not SEQ ID NO: 38.

In one or more embodiments of the membrane associated-ED protein, the ED domain amino acid sequence comprises at least 90% sequence similarity to SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, or SEQ ID NO: 30 In one or more embodiments, the resulting amino acid sequence of the membrane associated-ED protein is not SEQ ID NO: 38.

In one or more embodiments of the membrane associated-ED protein, the ED domain amino acid sequence comprises SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, or SEQ ID NO: 30. In one or more embodiments, the resulting amino acid sequence of the membrane associated-ED protein is not SEQ ID NO: 38.

In one or more embodiments, the first linker peptide has an amino sequence comprises at least 70% sequence similarity to SEQ ID NO: 143, SEQ ID NO: 144, SEQ ID NO: 145, SEQ ID NO: 146, SEQ ID NO: 147, SEQ ID NO: 148, SEQ ID NO: 149, SEQ ID NO: 150, SEQ ID NO: 151, SEQ ID NO: 152 or SEQ ID NO: 153.

In one or more embodiments, the first linker peptide has an amino sequence comprises at least 90% sequence similarity to SEQ ID NO: 143, SEQ ID NO: 144, SEQ ID NO: 145, SEQ ID NO: 146, SEQ ID NO: 147, SEQ ID NO: 148, SEQ ID NO: 149, SEQ ID NO: 150, SEQ ID NO: 151, SEQ ID NO: 152 or SEQ ID NO: 153.

In one or more embodiments, the first linker peptide has an amino sequence comprising SEQ ID NO: 143, SEQ ID NO: 144, SEQ ID NO: 145, SEQ ID NO: 146, SEQ ID NO: 147, SEQ ID NO: 148, SEQ ID NO: 149, SEQ ID NO: 150, SEQ ID NO: 151, SEQ ID NO: 152 or SEQ ID NO: 153.

In one or more embodiments of the membrane associated-ED protein, the membrane associated-ED amino acid sequence comprises at least 70% sequence similarity to SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 121, SEQ ID NO: 122, SEQ ID NO: 123, SEQ ID NO: 124, SEQ ID NO: 125, SEQ ID NO: 126, SEQ ID NO: 127, SEQ ID NO: 128, SEQ ID NO: 129, SEQ ID NO: 130, SEQ ID NO: 131, SEQ ID NO: 132, SEQ ID NO: 133, SEQ ID NO: 134, SEQ ID NO: 135, SEQ ID NO: 136, SEQ ID NO: 137, SEQ ID NO: 138, SEQ ID NO: 139, SEQ ID NO: 140, SEQ ID NO: 141 or SEQ ID NO: 142. In one or more embodiments, the membrane associated-ED amino acid sequence is not SEQ ID NO: 38.

In one or more embodiments of the membrane associated-ED protein, the membrane associated-ED amino acid sequence comprises at least 90% sequence similarity to SEQ ID NO:31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 121, SEQ ID NO: 122, SEQ ID NO: 123, SEQ ID NO: 124, SEQ ID NO: 125, SEQ ID NO: 126, SEQ ID NO: 127, SEQ ID NO: 128, SEQ ID NO: 129, SEQ ID NO: 130, SEQ ID NO: 131, SEQ ID NO: 132, SEQ ID NO: 133, SEQ ID NO: 134, SEQ ID NO: 135, SEQ ID NO: 136, SEQ ID NO: 137, SEQ ID NO: 138, SEQ ID NO: 139, SEQ ID NO: 140, SEQ ID NO: 141 or SEQ ID NO: 142. In one or more embodiments, the membrane associated-ED amino acid sequence is not SEQ ID NO: 38.

In one or more embodiments of the membrane associated-ED protein, the membrane associated-ED amino acid sequence comprises SEQ ID NO:31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 121, SEQ ID NO: 122, SEQ ID NO: 123, SEQ ID NO: 124, SEQ ID NO: 125, SEQ ID NO: 126, SEQ ID NO: 127, SEQ ID NO: 128, SEQ ID NO: 129, SEQ ID NO: 130, SEQ ID NO: 131, SEQ ID NO: 132, SEQ ID NO: 133, SEQ ID NO: 134, SEQ ID NO: 135, SEQ ID NO: 136, SEQ ID NO: 137, SEQ ID NO: 138, SEQ ID NO: 139, SEQ ID NO: 140, SEQ ID NO: 141 or SEQ ID NO: 142. In one or more embodiments, the membrane associated-ED amino acid sequence is not SEQ ID NO: 38.

In one or more embodiments, the membrane associated-ED protein further comprises a curvature sensing domain. In one or more embodiments, the curvature sensing domain is on the C-terminus or N-terminus of the membrane associated-ED protein. In one or more embodiments, the curvature sensing domain is a linker peptide between the signal domain and the ED domain.

In one or more embodiments, the N-terminus of the membrane associated-ED protein does not have methionine.

Another aspect of the present disclosure pertains to a polynucleotide comprising a nucleotide sequence encoding the membrane associated-ED protein.

Another aspect of the present disclosure pertains to a fusion protein comprising a vesicle targeting protein and a protein of interest. In one or more embodiments, the vesicle targeting protein comprises a signal domain and an effector domain (ED). In some embodiments, the signal domain comprises a lipid conjugating domain. In some embodiments, the vesicle targeting protein comprises the lipid conjugating domain directly linked to the effector domain (ED). In some embodiments, the vesicle targeting protein comprises the lipid conjugating domain linked to the effector domain (ED) by at least a second linker peptide. In one or more embodiments, the second linker peptide has an amino acid sequence comprising SEQ ID NO: 143, SEQ ID NO: 144, SEQ ID NO: 145, SEQ ID NO: 146, SEQ ID NO: 147, SEQ ID NO: 148, SEQ ID NO: 149, SEQ ID NO: 150, SEQ ID NO: 151, SEQ ID NO: 152 or SEQ ID NO: 153.

In one or more embodiments of the fusion protein, the vesicle targeting protein amino acid sequence comprises at least 70% sequence similarity to SEQ ID NO:31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 121, SEQ ID NO: 122, SEQ ID NO: 123, SEQ ID NO: 124, SEQ ID NO: 125, SEQ ID NO: 126, SEQ ID NO: 127, SEQ ID NO: 128, SEQ ID NO: 129, SEQ ID NO: 130, SEQ ID NO: 131, SEQ ID NO: 132, SEQ ID NO: 133, SEQ ID NO: 134, SEQ ID NO: 135, SEQ ID NO: 136, SEQ ID NO: 137, SEQ ID NO: 138, SEQ ID NO: 139, SEQ ID NO: 140, SEQ ID NO: 141 or SEQ ID NO: 142. In one or more embodiments, the vesicle targeting protein amino acid sequence is not SEQ ID NO: 38.

In one or more embodiments of the fusion protein, the vesicle targeting protein amino acid sequence comprises at least 90% sequence similarity to SEQ ID NO:31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 121, SEQ ID NO: 122, SEQ ID NO: 123, SEQ ID NO: 124, SEQ ID NO: 125, SEQ ID NO: 126, SEQ ID NO: 127, SEQ ID NO: 128, SEQ ID NO: 129, SEQ ID NO: 130, SEQ ID NO: 131, SEQ ID NO: 132, SEQ ID NO: 133, SEQ ID NO: 134, SEQ ID NO: 135, SEQ ID NO: 136, SEQ ID NO: 137, SEQ ID NO: 138, SEQ ID NO: 139, SEQ ID NO: 140, SEQ ID NO: 141 or SEQ ID NO: 142. In one or more embodiments, the vesicle targeting protein amino acid sequence is not SEQ ID NO: 38.

In one or more embodiments of the fusion protein, the vesicle targeting protein amino acid sequence comprises SEQ ID NO:31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 121, SEQ ID NO: 122, SEQ ID NO: 123, SEQ ID NO: 124, SEQ ID NO: 125, SEQ ID NO: 126, SEQ ID NO: 127, SEQ ID NO: 128, SEQ ID NO: 129, SEQ ID NO: 130, SEQ ID NO: 131, SEQ ID NO: 132, SEQ ID NO: 133, SEQ ID NO: 134, SEQ ID NO: 135, SEQ ID NO: 136, SEQ ID NO: 137, SEQ ID NO: 138, SEQ ID NO: 139, SEQ ID NO: 140, SEQ ID NO: 141 or SEQ ID NO: 142. In one or more embodiments, the vesicle targeting protein amino acid sequence is not SEQ ID NO: 38.

In one or more embodiments of the fusion protein, the signal domain comprises one or more of a lipid conjugation domain, e.g. a myristoylation signal domain, an ESCRT binding motif, an MIT motif, a palmitoylation signal domain, a prenylation signal domain, a lysosomal signal domain, a glycosylphosphatidylinsiton anchor protein and an immunoglobulin heavy-chain binding protein (BiP) secretion signal domain. In one or more embodiments, the lipid conjugating domain is a myristoylation signal domain.

In one or more embodiments of the fusion protein, the amino acid sequence of the signal domain has at least 70% similarity to the myristoylation signal domain of one or more of the MARCKS, MARCKSL1, SNX1 or BASP1 family of protein. In one or more embodiments of the fusion protein, the amino acid sequence of the signal domain has at least 90% similarity to the myristoylation signal domain of MARCKS, MARCKSL1 or BASP1 family of protein. In one or more embodiments of the fusion protein, the amino acid sequence of signal domain has 100% similarity to the myristoylation signal domain of MARCKS, MARCKSL1 or BASP1 family of protein.

In one or more embodiments of the fusion protein, the amino acid sequence of the signal domain of the membrane associated-ED protein comprises at least 70% sequence similarity to SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 155, SEQ ID NO: 156, SEQ ID NO: 157, SEQ ID NO: 158, SEQ ID NO: 159 or SEQ ID NO: 165.

In one or more embodiments of the fusion protein, the amino acid sequence of the signal domain of the vesicle targeting protein comprises at least 90% sequence similarity to SEQ ID NO:1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 155, SEQ ID NO: 156, SEQ ID NO: 157, SEQ ID NO: 158, SEQ ID NO: 159 or SEQ ID NO: 165.

In one or more embodiments of the fusion protein, the amino acid sequence of the signal domain of the vesicle targeting protein comprises SEQ ID NO:1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 155, SEQ ID NO: 156, SEQ ID NO: 157, SEQ ID NO: 158, SEQ ID NO: 159 or SEQ ID NO: 165.

In one or more embodiments of the fusion protein, the amino acid sequence of the ED domain of the vesicle targeting protein comprises at least 70% sequence similarity to SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, or SEQ ID NO: 30.

In one or more embodiments of the fusion protein, the amino acid sequence of the ED domain of the vesicle targeting protein comprises at least 90% sequence similarity to SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, or SEQ ID NO: 30.

In one or more embodiments of the fusion protein, the amino acid sequence of the ED domain of the vesicle targeting protein comprises SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, or SEQ ID NO: 30.

In one or more embodiments of the fusion protein, the vesicle targeting protein comprises Nef protein or a variant thereof.

In one or more embodiments of the fusion protein, the protein of interest is a therapeutic protein.

In one or more embodiments of the fusion protein, the therapeutic protein comprises a lysosomal protein.

In one or more embodiments of the fusion protein, the therapeutic protein comprises CDKL5, alpha-galactosidase, β-galactosidase, β-hexosaminidase, galactosylceramidase, arylsulfatase, β-glucocerebrosidase, glucocerebrosidase, lysosomal acid lipase, lysosomal enzyme acid sphingomyelinase, formylglycine-generating enzyme, iduronidase, acetyl-CoA:alpha-glucosaminide N-acetyltransferase, glycosaminoglycan alpha-L-iduronohydrolase, heparan N-sulfatase, N-acetyl-α-D-glucosaminidase (NAGLU), iduronate-2-sulfatase, galactosamine-6-sulfate sulfatase, N-acetylgalactosamine-6-sulfatase, glycosaminoglycan N-acetylgalactosamine 4-sulfatase, β-glucuronidase, hyaluronidase, alpha-N-acetyl neuraminidase (sialidase), ganglioside sialidase, phosphotransferase, alpha-glucosidase, alpha-D-mannosidase, beta-D-mannosidase, aspartylglucosaminidase, alpha-L-fucosidase, battenin, palmitoyl protein thioesterases, and other Batten-related proteins, or an enzymatically active fragment thereof.

In one or more embodiments of the fusion protein, the Batten-related protein of the therapeutic protein comprises ceroid-lipofuscinosis neuronal protein 1, ceroid-lipofuscinosis neuronal protein 2, ceroid-lipofuscinosis neuronal protein 3, ceroid-lipofuscinosis neuronal protein 4, ceroid-lipofuscinosis neuronal protein 5, ceroid-lipofuscinosis neuronal protein 6, ceroid-lipofuscinosis neuronal protein 7, ceroid-lipofuscinosis neuronal protein 8, ceroid-lipofuscinosis neuronal protein 19, ceroid-lipofuscinosis neuronal protein 10, ceroid-lipofuscinosis neuronal protein 11, ceroid-lipofuscinosis neuronal protein 12, ceroid-lipofuscinosis neuronal protein 13 and ceroid-lipofuscinosis neuronal protein 14.

In one or more embodiments of the fusion protein, the therapeutic protein comprises one or more hormones and/or growth and/or differentiation factors.

In one or more embodiments of the fusion protein, the hormones and/or growth and/or differentiation factors of the therapeutic protein comprises one or more of insulin, glucagon, growth hormone (GH), parathyroid hormone (PTH), growth hormone releasing factor (GRF), follicle stimulating hormone (FSH), luteinizing hormone (LH), human chorionic gonadotropin (hCG), vascular endothelial growth factor (VEGF), angiopoietins, angiostatin, granulocyte colony stimulating factor (GCSF), erythropoietin (EPO), connective tissue growth factor (CTGF), basic fibroblast growth factor (bFGF), acidic fibroblast growth factor (aFGF), epidermal growth factor (EGF), platelet-derived growth factor (PDGF), insulin growth factors I (IGF-I), insulin growth factors II (IGF-II), transforming growth factor, heregluin growth factor, neuregulin growth factor, ARIA growth factor, neu differentiation factor (NDF), nerve growth factor (NGF), brain-derived neurotrophic factor (BDNF), neurotrophins NT-3, neurotrophins NT-4/5, ciliary neurotrophic factor (CNTF), glial cell line derived neurotrophic factor (GDNF), neurturin, agrin, semaphorins, collapsins, netrin-1, netrin-2, hepatocyte growth factor (HGF), ephrins, noggin, sonic hedgehog, or tyrosine hydroxylase.

In one or more embodiments of the fusion protein, the transforming growth factor of the hormones and/or growth and/or differentiation factors of the therapeutic protein comprises TGFα, activins, inhibins, bone morphogenic proteins (BMP-1), bone morphogenic proteins (BMP-2), bone morphogenic proteins (BMP-3), bone morphogenic proteins (BMP-4), bone morphogenic proteins (BMP-5), bone morphogenic proteins (BMP-6), bone morphogenic proteins (BMP-7), bone morphogenic proteins (BMP-8), bone morphogenic proteins (BMP-9), bone morphogenic proteins (BMP-10), bone morphogenic proteins (BMP-11), bone morphogenic proteins (BMP-12), bone morphogenic proteins (BMP-13), bone morphogenic proteins (BMP-14), or bone morphogenic proteins (BMP-15).

In one or more embodiments of the fusion protein, the therapeutic protein comprises one or more immune system regulatory proteins.

In one or more embodiments of the fusion protein, the immune system regulatory protein of the therapeutic protein comprises cytokines and lymphokines, wherein cytokines and lymphokines comprises thrombopoietin (TPO), interleukins-1 (IL-1), interleukins-2 (IL-2), interleukins-3 (IL-3), interleukins-4 (IL-4), interleukins-5 (IL-5), interleukins-6 (IL-6), interleukins-7 (IL-7), interleukins-8 (IL-8), interleukins-9 (IL-9), interleukins-10 (IL-10), interleukins-11 (IL-11), interleukins-12 (IL-12), interleukins-13 (IL-13), interleukins-14 (IL-14), interleukins-15 (IL-15), interleukins-16 (IL-16), interleukins-17 (IL-17), interleukins-18 (IL-18), interleukins-19 (IL-19), interleukins-20 (IL-20), interleukins-21 (IL-21), interleukins-22 (IL-22), interleukins-23 (IL-23), interleukins-24 (IL-24), interleukins-25 (IL-25), monocyte chemoattractant protein, leukemia inhibitory factor, granulocyte-macrophage colony stimulating factor, Fas ligand, tumor necrosis factors α, tumor necrosis factors β, interferons α, β, and γ, stem cell factor, or flk-2/flt3 ligand.

In one or more embodiments of the fusion protein, the therapeutic protein comprises one or more immunoglobulins IgG, IgM, IgA, IgD and IgE, chimeric immunoglobulins, humanized antibodies, single chain antibodies, T cell receptors, chimeric T cell receptors, single chain T cell receptors, class I MHC molecules, class II MHC molecules, engineered immunoglobulins, or engineered MHC molecules.

In one or more embodiments of the fusion protein, the therapeutic protein comprises one or more complement regulatory proteins, wherein the complement regulatory proteins comprises membrane cofactor protein (MCP), decay accelerating factor (DAF), CR1, CF2, or CD59.

In one or more embodiments of the fusion protein, the therapeutic protein comprises on or more receptors for the hormones, growth factors, cytokines, lymphokines, regulatory proteins, or immune system proteins.

In one or more embodiments of the fusion protein, the therapeutic protein comprises one or more receptors for cholesterol regulation and/or lipid modulation, including the low density lipoprotein (LDL) receptor, high density lipoprotein (HDL) receptor, the very low density lipoprotein (VLDL) receptor, or scavenger receptors.

In one or more embodiments of the fusion protein, the therapeutic protein comprises one or more members of the steroid hormone receptor superfamily including glucocorticoid receptors and estrogen receptors, Vitamin D receptors, or other nuclear receptors.

In one or more embodiments of the fusion protein, the therapeutic protein comprises one or more transcription factors including jun, fos, max, mad, serum response factor (SRF), AP-1, AP2, myb, MyoD and myogenin, ETS-box containing proteins, TFE3, E2F, ATF1, ATF2, ATF3, ATF4, ZFS, NFAT, CREB, HNF-4, C/EBP, SP1, CCAAT-box binding proteins, interferon regulation factor (IRF-1), Wilms tumor protein, ETS-binding protein, STAT, GATA-box binding proteins, or the forkhead family of winged helix proteins

In one or more embodiments of the fusion protein, the therapeutic protein comprises one or more of carbamoyl synthetase I, ornithine transcarbamylase, arginosuccinate synthetase, arginosuccinate lyase, arginase, fumarylacetacetate hydrolase, phenylalanine hydroxylase, alpha-1 antitrypsin, glucose-6-phosphatase, porphobilinogen deaminase, cystathione beta-synthase, branched chain ketoacid decarboxylase, albumin, isovaleryl-coA dehydrogenase, propionyl CoA carboxylase, methyl malonyl CoA mutase, glutaryl CoA dehydrogenase, insulin, beta-glucosidase, pyruvate carboxylate, hepatic phosphorylase, phosphorylase kinase, glycine decarboxylase, H-protein, T-protein, a cystic fibrosis transmembrane regulator (CFTR) sequence, and a dystrophin gene product.

In one or more embodiments of the fusion protein, the therapeutic protein comprises one or more proteins used for treatment of hemophilia, including hemophilia B and hemophilia A.

In one or more embodiments of the fusion protein, the therapeutic protein comprises one or more non-naturally occurring polypeptides, such as chimeric or hybrid polypeptides having a non-naturally occurring amino acid sequence containing insertions, deletions or amino acid substitutions.

In one or more embodiments of the fusion protein, the therapeutic protein comprises the non-naturally occurring polypeptide comprises single-chain engineered immunoglobulins, antisense molecules and catalytic nucleic acids.

In one or more embodiments of the fusion protein, the therapeutic protein comprises one or more proteins that are useful for treating an individual suffering from autoimmune disease and disorder by conferring a broad based protective immune response against target that is associated with autoimmunity including cell receptor and cell which produce “self”-directed antibody.

In one or more embodiments of the fusion protein, the cell receptor of the therapeutic protein comprises T cell receptor.

In one or more embodiments of the fusion protein, the autoimmune disease comprises Rheumatoid arthritis (RA), multiple sclerosis (MS), Sjögren's syndrome, sarcoidosis, insulin dependent diabetes mellitus (IDDM), autoimmune thyroiditis, reactive arthritis, ankylosing spondylitis, scleroderma, polymyositis, dermatomyositis, psoriasis, vasculitis, Wegener's granulomatosis, Crohn's disease and ulcerative colitis.

In one or more embodiments, the vesicle targeting protein targets one or more of an exosome or an extracellular vesicle.

In one or more embodiments, the fusion protein comprises one or more of an affinity tag, a linker peptide, protease cleavage sites, a secretion signal peptide, cellular targeting domains, reporters, enzymes, or combination thereof.

In one or more embodiments, the fusion protein comprises one or more curvature sensing domains.

In one or more embodiments, the fusion protein further comprising a third linker peptide, wherein the third linker peptide is located between the vesicle targeting protein and the protein of interest or the terminal end of fusion protein. In some embodiments, the third linker peptide comprises one or more of a spacer peptide, a transmembrane domain and extravesicular domain.

In one or more embodiments, the N-terminus of fusion protein does not have methionine.

Another aspect of the disclosure includes a nucleotide sequence encoding the fusion protein as described herein.

In one or more embodiments of the fusion protein, the fusion protein is conjugated with one or more lipids. In one or more embodiments of the fusion protein, the fusion protein is conjugated by myristoylation. In some embodiments of the fusion protein, the fusion protein is conjugated by palmitoylation. In one or more embodiments of the fusion protein, the fusion protein is conjugated by prenylation. In some embodiments of the fusion protein, the fusion protein is conjugated by a glycosylphosphatidylinositol anchor protein.

Another aspect of the present disclosure pertains to reconstituting the fusion protein into a lipid vesicle.

Another aspect of the present disclosure pertains to a method of producing the fusion protein comprises expressing the fusion protein, isolating exosomes, and purifying the fusion protein.

In one or more embodiments, the method of producing the fusion protein further comprises lipid conjugation. In some embodiments of the method of producing the fusion protein, wherein lipid conjugation is performed by myristoylation. In one or more embodiments of the method of producing the fusion protein, wherein lipid conjugation is performed by palmitoylation. In some embodiments of the method of producing the fusion protein, wherein lipid conjugation is performed by prenylation. In one or more embodiments of the method of producing the fusion protein, wherein lipid conjugation is performed by glycosylphosphatidylinositol anchor protein.

In one or more embodiments of the method of producing the fusion protein, the method further includes reconstituting the fusion protein into a lipid vesicle.

In one or more embodiments of the method of producing the fusion protein, the fusion protein is expressed in mesenchymal stem cells, multiple myeloma cells, Expi293F cells, PC12 cells, COST cell, HAP1 cells, Chinese hamster ovary (CHO) cells, HeLa cells, human embryonic kidney (HEK) cells, mouse primary myoblasts, NIH 3T3 cells, Escherichia coli cells, or a variant thereof.

Another aspect of the present disclosure pertains to a pharmaceutical formulation comprises the fusion protein and a pharmaceutically acceptable carrier.

Another aspect of the present disclosure pertains to a method of treating a disease or disorder comprises administering the pharmaceutical formulation to a patient in need thereof. In one or more embodiments of the method of treating a disease or disorder, the pharmaceutical formulation is administered via one or more of intrathecally, intracranially, intra cisterna magnally, intravenously, intracisternally, intracerebroventrically, or intraparenchymally.

Another aspect of the present disclosure pertains to a gene therapy composition comprises a gene therapy delivery system and the polynucleotide encoding an engineered protein. The engineered protein comprises a vesicle targeting protein and a protein of interest. In some embodiments, the engineered protein is the fusion protein.

In one or more embodiments of the gene therapy composition, the gene therapy delivery system comprises one or more of Clustered Regularly Interspaced Short Palindromic Repeat (CRISPR) associated protein 9 (CRISPR-Cas-9), Transcription activator-like effector nuclease (TALEN), or ZNF (Zinc finger protein).

In one or more embodiments of the gene therapy composition, the gene therapy delivery system comprises one or more of a vector, a liposome, a lipid-nucleic acid nanoparticle, an exosome, and a gene editing system.

In one or more embodiments of the gene therapy composition, the gene therapy delivery system comprises a viral vector.

In one or more embodiments of the gene therapy composition, the viral vector comprises one or more of an adenoviral vector, an adeno-associated viral vector, a lentiviral vector, a retroviral vector, a poxviral vector, or a herpes simplex viral vector.

In one or more embodiments of the gene therapy composition, the viral vector comprises a viral polynucleotide operably linked to the polynucleotide encoding the engineered protein.

In one or more embodiments of the gene therapy composition, the viral vector comprises at least one inverted terminal repeat (ITR).

In one or more embodiments of the gene therapy composition, the viral vector comprises one or more of an SV40 intron, a polyadenylation signal, or a stabilizing element.

In one or more embodiments of the gene therapy composition, the gene therapy delivery system comprises a promoter.

In one or more embodiments of the gene therapy composition, the gene therapy delivery system comprises a polynucleotide encoding a leader signal polypeptide.

Another aspect of the present disclosure pertains to a gene therapy composition formulation comprises a pharmaceutically acceptable carrier and the gene therapy composition.

Another aspect of the present disclosure pertains to a method of treating a disease or disorder comprises administering the gene therapy composition formulation to a patient in need thereof.

Another aspect of the present disclosure pertains to a method of preventing a disease or disorder comprising prophylactically administering the gene therapy composition formulation to a subject.

In one or more embodiments of the method of treating a disease or disorder, the gene therapy composition is administered via one or more of intrathecally, intravenously, intracisternally, intracerebroventrically, or intraparenchymally.

In one or more embodiments, the gene therapy composition is administered orally, parenterally, sublingually, transdermally, rectally, transmucosally, topically, via inhalation, via buccal administration, intrapleurally, intravenous, intraarterial, intraperitoneal, subcutaneous, intramuscular, intranasal, intrathecal, and intraarticular or combinations thereof.

### BRIEF DESCRIPTION OF DRAWINGS

FIG. 1. MARCKS truncations and MyrED schematic.

FIG. 2. Truncated MARCKS variants and MyrEd targeted to exosomes. COS-7 cells were transfected with plasmids encoding one of truncated MARCKS variants or MyrED fused to fluorescent protein mCherry. The transfected cells were immune-visualized using mCherry (left) and Anti-CD63 antibody directed against exosomal protein CD63 (right).

FIG. 3. Cross-correction using MARCKS variant or MyrED. Cos-7 cells were transfected with plasmids encoding mCherry fluorescence protein fused to mMARCKS-Through ED, Myr-mMARCKS-ED, or BiPa. The transfected Cos-7 cells (white) were co-cultured with non-transfected cells (black).

FIG. 4. Cross-correction in DIV15 primary cortical neuron cells. Cos-7 cells were transfected with plasmids encoding mCherry fluorescence protein fused to mMARCKS-Through ED, Myr-mMARCKS-ED, or BiPa. The transfected cells (white) were co-cultured with non-transfected DIV15 primary cortical neuron cells (black).

FIG. 5. Sequence alignment of MyrED variants. Alignment of the various MyrED sequences developed for this disclosure. myristoylation signal region, SNX1, and MARCKS or MLP ED domains are shaded.

FIG. 6. MyrED variants targeting exosomes in 48 hours. Expi293F cells were transfected a polynucleotide encoding MyrGagOnly, MyrED6, MyrED7, MyrED8, MyrED9, or 445-MARCKS-ED, fused with mCherry. FIG. 6A shows relative fluorescence in conditioned media after 24 and 48 hours. FIG. 6B shows relative fluorescence in purified exosomes. FIG. 6C shows western blot analysis of purified exosomes with primary antibody against mCherry.

FIG. 7. MyrED variants targeting exosomes in 96 hours. Expi293F cells were transfected a polynucleotide encoding MyrED6, MyrED7, MyrED8, MyrED10, MyrED11, MyrED12, MyrED13, MyrED14, MyrED15, MyrED16, MyrED17, MyrED18, MyrED19, MyrED20, MyrED21, MyrED22, MyrED23, MyrED24, MyrED25, or MyrED26, fused with mCherry. FIG. 7A shows relative fluorescence in conditioned media after 48 and 96 hours.

FIG. 7B shows relative fluorescence in purified exosomes. FIG. 7C shows co-localization of MyrED13 or MyrED17 fused mCherry protein with CD63 protein in Cos-7 cells.

FIG. 8. Application of MyrEDs to GAA and CDKL5. Constructs encoding MyrED13-GAA-HPC4 protein was used to transfect Cos7 cells. FIG. 8A shows co-localization of MyrED13-GAA-HPC4 protein and CD63 protein is Cos-7. Constructs encoding GAA protein fused with MyrED13, MyrED14 and MyrED17 were used to transfect the cells.

FIG. 8B shows western blot analysis of fusion proteins, MyrED13-GAA, MyrED14-GAA and MyrED17-GAA, targeting exosomes. Similarly, constructs encoding CDKL5 protein fused with MyrED13, MyrED14 and MyrED17 were used to transfect the cells. FIG. 8C shows western blot analysis of fusion proteins, MyrED13-CDKL5, MyrED14-CDKL5 and MyrED17-CDKL5 targeting exosomes.

FIG. 9. Western blot analysis of mCherry and GFP presence in liver. Levels of mCherry, GFP, and GAPDH in liver were analyzed using western blot analysis.

FIG. 10. Fluorescent image analysis of MyrED27 transduced embryonic brain cells. Brain sections from embryos transduced with MyrED27 were fluorescently imaged to determine transduction efficiency. GFP is expressed in only transfected cells. Similarly, cross-corrected cells only express mCherry. Accordingly, FIG. 10A shows GFP transfected cells, FIG. 10B shows mCherry cross-corrected cells and FIG. 10C shows a merge image thereof.

FIG. 11. Fluorescent image analysis of MyrED77 transduced embryonic brain cells. Brain sections from embryos transduced with MyrED77 were fluorescently imaged to determine transduction efficiency. GFP is expressed in only transfected cells. Similarly, cross-corrected cells only express mCherry. Accordingly, FIG. 11A shows GFP transfected cells, FIG. 11B shows mCherry cross-corrected cells and FIG. 11C shows a merge image thereof.

FIG. 12. Fluorescent image analysis of MyrED81 transduced embryonic brain cells. Brain sections from embryos transduced with MyrED81 were fluorescently imaged to determine transduction efficiency. GFP is expressed in only transfected cells. Similarly, cross-corrected cells only express mCherry. Accordingly, FIG. 12A shows GFP transfected cells, FIG. 12B shows mCherry cross-corrected cells and FIG. 12C shows a merge image thereof.

FIG. 13. Fluorescent image analysis of MyrED88 transduced embryonic brain cells. Brain sections from embryos transduced with MyrED88 were fluorescently imaged to determine transduction efficiency. GFP is expressed in only transfected cells. Similarly, cross-corrected cells only express mCherry. Accordingly, FIG. 13A shows GFP transfected cells, FIG. 13B shows mCherry cross-corrected cells and FIG. 13C shows a merge image thereof.

FIG. 14. Comparative transduction efficiency analysis. Brain sections from embryos transduced with the fusion proteins, MyrED27, MyrED77, MyrED81 and MyrED88, were fluorescently imaged and compared with a positive control BiP-mCherry to determine relative transduction efficiency. GFP is expressed in only transfected cells. Similarly, cross-corrected cells only express mCherry. Accordingly, FIGS. 14A, 14D, 14G, 14J and 14M show GFP transfected cells, FIGS. 14B, 14E, 14H, 14K and 14N show mCherry cross-corrected cells, and FIGS. 14C, 14F, 14I, 14L and 14O show merge images thereof.

FIG. 15. CDKL5 Targeting with MyrED53-67. FIG. 15A shows a comparison on the components for MyrED27 and MyrED54-67. FIG. 15B is a western blot showing CDKL5 expression (˜115 kDa) in various exosome targeted variants.

FIG. 16. TPP1 Targeting with MyrED59-65. FIG. 16 is a western blot showing TPP1 expression (˜75 kDa) and CD81 expression (Tetraspanin marker) in various exosome targeted variants.

FIG. 17. Testing of C-Terminal Exosome Targeting Tags. FIG. 17A shows a comparison on the components for MyrED81-94. FIG. 17B shows BiP-mCherry-MyrED exosomes normalized to volume for various exosome targeted variants.

FIG. 18. Testing of C-Terminal Exosome Targeting Tags. FIG. 18 shows mCherry fluorescence screen in various C terminally tagged exosome targeted variants.

FIG. 19. GAA Targeting with MyrED. FIG. 19 is a western blot showing GAA (˜105 kDa) and CD9 (˜20 kDa) Tetraspanin marker expression in C-terminally tagged exosome targeted variants

FIG. 20. NAGLU Targeting with MyrED. FIG. 20 is a western blot showing NAGLU (˜85 kDa) and CD9 (˜20 kDa) Tetraspanin marker expression in C-terminally tagged exosome targeted variants. COV2 NAGLU (T343P/A181L) was regarded as negative control.

FIG. 21. TTP1Targeting with MyrED. FIG. 21 is a western blot showing TPP1 (HPC4 Ab-˜65 kDa) and CD9 (˜20 kDa) Tetraspanin marker expression in C-terminally tagged exosome targeted variants. BiP1 tagged TPP1 construct was regarded as negative control.

### DETAILED DESCRIPTION

Before describing several exemplary embodiments of the disclosure, it is to be understood that the disclosure is not limited to the details of construction or process steps set forth in the following description. The disclosure is capable of other embodiments and of being practiced or being carried out in various ways.

Various embodiments of the present disclosure relate delivering a therapeutic molecule to a cell via vesicles such as exosomes.

As described herein, exosomes or other extracellular vesicles may be used for gene delivery. Exosomes are small membrane-bound vesicles (30-100 nm) of endocytic origin that are released into the extracellular environment following fusion of multivesicular bodies with the plasma membrane. It is known that the patient may produce an immune response to a protein delivered by enzyme replacement therapy, potentially impacting the efficacy of the therapy (Harmatz, Clin Ther, 2015). When high levels of secreted proteins foreign to the body are delivered to the bloodstream, often neutralizing antibodies against the protein develop. This is especially true in patients with no residual enzyme expression (cross-reactive immunological material (CRIM)—negative patients), to such a degree that it results in poorer rates of survival and clinical outcomes (Kishnani et al., Mol Genet Metab., 2010). Presumably, this is the case for gene therapy-delivered protein as well. Exosomes serve as a “trojan horse” carrying a protein payload. The protein is concealed within the vesicle, so it is unable to interact with immune cells and antibodies. Exosomes are already naturally secreted by cells in the body so it is unlikely they will induce an immune response. By targeting proteins to exosomes, the proteins are less likely to induce an immune response, thereby enhancing the efficacy of the therapy.

Exosome production has been described for many immune cells including B cells, T cells, and dendritic cells (DCs). Exosomes derived from B lymphocytes and mature DCs express MHC-II, MHC-I, CD86 and ICAM-1, and have been used to induce specific anti-tumor T cytotoxic responses and anti-tumor immunity in experimental models and clinical trials. The potential of exosome-mediated gene delivery has been shown with delivery of murine mRNAs and miRNAs to human mast cells and glioma-derived exosomes have been demonstrated to transfer mRNAs produced by exogenous DNA plasmids to heterologous cells, but loading and delivery of exogenous DNA, siRNAs and other modified oligonucleotides has not been demonstrated as yet.

Additionally, current approaches of in vitro packing drug products into exosomes is highly challenging, including the high cost of exosome manufacturing and relatively low efficiency in drug incorporation. Exosomes are inherently heterogeneous and current knowledge about their contents is poor; thus, raising safety concerns especially when exosomes are manufactured in vitro from immortalized cells. There is evidence that individual tissues may internalize only exosomes originated in the same tissue, therefore in vitro manufactured exosomes may not efficiently target all tissues/organs.

In various embodiments, the methods and/or compositions disclosed herein improve the delivery of therapeutic cargo via vesicles such as exosomes.

### Definitions

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

The terms “coding sequence” or “encoding nucleic acid” refer to the nucleic acids (RNA or DNA molecule) that comprise a nucleotide sequence which encodes a protein. The coding sequence can further include initiation and termination signals operably linked to regulatory elements including a promoter and polyadenylation signal capable of directing expression in the cells of an individual or mammal to which the nucleic acid is administered.

The terms “complement” or “complementary” refer to Watson-Crick (e.g., A-T/U and C-G) or Hoogsteen base pairing between nucleotides or nucleotide analogs of nucleic acid molecules.

The term “directly linked to” refers to a condition where two peptides are linked through an amide linkage with no amino acid between the two peptides.

The term “expressible form” refers to gene constructs that contain the necessary regulatory elements operably linked to a coding sequence that encodes a target protein, such that when present in the cell of the individual, the coding sequence will be expressed.

The term “fragment” refers to a nucleotide sequence or a portion thereof that encodes a polypeptide that maintains a substantial level of function in a mammal. The fragments can be DNA fragments selected from at least one of the various nucleotide sequences that encode protein fragments set forth below.

The term “genetic construct” refers to the DNA or RNA molecules that comprise a nucleotide sequence which encodes a protein. The coding sequence includes initiation and termination signals operably linked to regulatory elements including a promoter and polyadenylation signal capable of directing expression in the cells of the individual to whom the nucleic acid molecule is administered. As used herein, the term “expressible form” refers to gene constructs that contain the necessary regulatory elements operable linked to a coding sequence that encodes a protein such that when present in the cell of the individual, the coding sequence will be expressed.

The term “identical” or “identity” as used herein in the context of two or more nucleic acids or polypeptide sequences, means that the sequences have a specified percentage of residues that are the same over a specified region. The percentage can be calculated by optimally aligning the two sequences, comparing the two sequences over the specified region, determining the number of positions at which the identical residue occurs in both sequences to yield the number of matched positions, dividing the number of matched positions by the total number of positions in the specified region, and multiplying the result by 100 to yield the percentage of sequence similarity. In cases where the two sequences are of different lengths or the alignment produces one or more staggered ends and the specified region of comparison includes only a single sequence, the residues of single sequence are included in the denominator but not the numerator of the calculation. When comparing DNA and RNA, thymine (T) and uracil (U) can be considered equivalent. Identity can be performed manually or by using a computer sequence algorithm such as BLAST or BLAST 2.0.

The term “gene therapy delivery system” refers to any system that can be used to deliver an exogenous gene of interest to a target cell so that the gene of interest will be expressed or overexpressed in the target cell. In one or more embodiments, the target cell is an in vivo patient cell. In one or more embodiments, the target cell is an ex vivo cell and the cell is then administered to the patient.

The term “host cell” means any cell of any organism that is selected, modified, transformed, grown, or used or manipulated in any way, for the production of a substance by the cell, for example the expression by the cell of a gene, a DNA or RNA sequence, a protein or an enzyme.

The term “minigene” refers to the combination of the transgene, promoter/enhancer, and 5′ and 3′ AAV ITRs is referred to as a “minigene” for ease of reference herein. Provided with the teachings of this disclosure, the design of such a minigene can be made by resort to conventional techniques.

The terms “nucleic acid” or “oligonucleotide” or “polynucleotide” refer to at least two nucleotides covalently linked together. The depiction of a single strand also defines the sequence of the complementary strand. Thus, a nucleic acid also encompasses the complementary strand of a depicted single strand. Many variants of a nucleic acid can be used for the same purpose as a given nucleic acid. Thus, a nucleic acid also encompasses substantially identical nucleic acids and complements thereof. A single strand provides a probe that can hybridize to a target sequence under stringent hybridization conditions. Thus, a nucleic acid also encompasses a probe that hybridizes under stringent hybridization conditions.

The term “operably linked” refers to the expression of a gene that is under the control of a promoter with which it is spatially connected. A promoter can be positioned 5′ (upstream) or 3′ (downstream) of a gene under its control. The distance between the promoter and a gene can be approximately the same as the distance between that promoter and the gene it controls in the gene from which the promoter is derived. As is known in the art, variation in this distance can be accommodated without loss of promoter function.

The term “patient” refers to any mammalian subject for whom diagnosis, treatment, or therapy is desired, particularly humans. The mammalian subject for purposes of treatment refers to any animal classified as a mammal, including humans, domestic and farm animals, and laboratory, zoo, sports, or pet animals, such as dogs, horses, cats, cows, sheep, goats, pigs, mice, rats, rabbits, guinea pigs, monkeys etc. The mammalian subject may be a fetus, a neonate, child, juvenile or an adult with disorder. In one or more embodiments, the mammalian subject is human.

The terms “peptide,” “protein,” or “polypeptide” refer to a linked sequence of amino acids and can be natural, synthetic, or a modification or combination of natural and synthetic.

The term “pharmaceutically acceptable” refers to molecular entities and compositions that are physiologically tolerable and do not typically produce untoward reactions when administered to a human. Preferably, as used herein, the term “pharmaceutically acceptable” means approved by a regulatory agency of the Federal or a state government or listed in the U.S. Pharmacopoeia or other generally recognized pharmacopoeia for use in animals, and more particularly in humans. The term “carrier” refers to a diluent, adjuvant, excipient, or vehicle with which the compound is administered. Such pharmaceutical carriers can be sterile liquids, such as water and oils. Water or aqueous solution saline solutions and aqueous dextrose and glycerol solutions are preferably employed as carriers, particularly for injectable solutions. Suitable pharmaceutical carriers are described in “Remington's Pharmaceutical Sciences” by E. W. Martin, 18th Edition, or other editions.

The term “promoter” refers to a site on DNA to which the enzyme RNA polymerase binds and initiates the transcription of DNA into RNA. A promoter can be a synthetic or naturally-derived molecule which is capable of conferring, activating or enhancing expression of a nucleic acid in a cell. A promoter can comprise one or more specific transcriptional regulatory sequences to further enhance expression and/or to alter the spatial expression and/or temporal expression of same. A promoter can also comprise distal enhancer or repressor elements, which can be located as much as several thousand base pairs from the start site of transcription. A promoter can be derived from sources including viral, bacterial, fungal, plants, insects, and animals. A promoter can regulate the expression of a gene component constitutively or differentially with respect to cell, the tissue or organ in which expression occurs or, with respect to the developmental stage at which expression occurs, or in response to external stimuli such as physiological stresses, pathogens, metal ions, or inducing agents. Representative examples of promoters include the bacteriophage T7 promoter, bacteriophage T3 promoter, SP6 promoter, lac operator-promoter, tac promoter, SV40 late promoter, SV40 early promoter, RSV-LTR promoter, CMV IE promoter, SV40 early promoter or SV40 late promoter and the CMV IE promoter.

The term “protein replacement therapy” is intended to refer to the introduction of an exogenous, purified protein into an individual having a deficiency in such protein. The administered protein can be obtained from natural sources or by recombinant expression. The term also refers to the introduction of a purified protein in an individual otherwise requiring or benefiting from administration of a purified protein. In at least one embodiment, such an individual suffers from protein deficiency. The introduced protein may be a purified, recombinant protein produced in vitro, or a protein purified from isolated tissue or fluid, such as, for example, placenta or animal milk, or from plants.

The term “subject” refers to a mammal that is capable of being administered the compositions described herein. The mammal can be, for example, a human, chimpanzee, dog, cat, horse, cow, mouse, or rat. In one or more embodiments, the subject is a human.

The term “substantial homology” or “substantial similarity,” when referring to a nucleic acid, or fragment thereof, indicates that, when optimally aligned with appropriate nucleotide insertions or deletions with another nucleic acid (or its complementary strand), there is nucleotide sequence similarity in at least about 95 to 99% of the aligned sequences. Preferably, the homology is over full-length sequence, or an open reading frame thereof, or another suitable fragment which is at least 15 nucleotides in length. Examples of suitable fragments are described herein.

The terms “sequence similarity” “sequence identity” “percent sequence identity” or “percent identical” in the context of nucleic acid sequences refers to the residues in the two sequences which are the same when aligned for maximum correspondence. Similarly, “percent sequence identity” may be readily determined for amino acid sequences, over the full-length of a protein, or a fragment thereof.

The term “serotype” is a distinction with respect to an AAV having a capsid which is serologically distinct from other AAV serotypes. Serologic distinctiveness is determined on the basis of the lack of cross-reactivity between antibodies to the AAV as compared to other AAV.

Cross-reactivity is typically measured in a neutralizing antibody assay. For this assay polyclonal serum is generated against a specific AAV in a rabbit or other suitable animal model using the adeno-associated viruses. In this assay, the serum generated against a specific AAV is then tested in its ability to neutralize either the same (homologous) or a heterologous AAV. The dilution that achieves 50% neutralization is considered the neutralizing antibody titer. If for two AAVs the quotient of the heterologous titer divided by the homologous titer is lower than 16 in a reciprocal manner, those two vectors are considered as the same serotype. Conversely, if the ratio of the heterologous titer over the homologous titer is 16 or more in a reciprocal manner the two AAVs are considered distinct serotypes.

As defined herein, to form serotype 9, antibodies generated to a selected AAV capsid must not be cross-reactive with any of AAV 1, AAV2, AAV3, AAV4, AAV5, AAV6, AAV7 or AAV8. In one embodiment, the AAV capsid is of a novel serotype, identified herein, as human AAV serotype 9.

The term “therapeutic protein” refers to a protein that is capable of replacing a defective or deficient protein associated with a genetic disorder in a patient having a genetic disorder.

The therapeutic protein, as used herein, can comprise hormones and/or growth and/or differentiation factors including, without limitation, insulin, glucagon, growth hormone (GH), parathyroid hormone (PTH), growth hormone releasing factor (GRF), follicle stimulating hormone (FSH), luteinizing hormone (LH), human chorionic gonadotropin (hCG), vascular endothelial growth factor (VEGF), angiopoietins, angiostatin, granulocyte colony stimulating factor (GCSF), erythropoietin (EPO), connective tissue growth factor (CTGF), basic fibroblast growth factor (bFGF), acidic fibroblast growth factor (aFGF), epidermal growth factor (EGF), platelet-derived growth factor (PDGF), insulin growth factors I and II (IGF-I and IGF-II), any one of the transforming growth factor α superfamily, including TGFα, activins, inhibins, or any of the bone morphogenic proteins (BMP) BMPs 1-15, any one of the heregluin/neuregulin/ARIA/neu differentiation factor (NDF) family of growth factors, nerve growth factor (NGF), brain-derived neurotrophic factor (BDNF), neurotrophins NT-3 and NT-4/5, ciliary neurotrophic factor (CNTF), glial cell line derived neurotrophic factor (GDNF), neurturin, agrin, any one of the family of semaphorins/collapsins, netrin-1 and netrin-2, hepatocyte growth factor (HGF), ephrins, noggin, sonic hedgehog and tyrosine hydroxylase.

The therapeutic protein, as used herein, can also comprise proteins that regulate the immune system including, without limitation, cytokines and lymphokines such as thrombopoietin (TPO), interleukins (IL) IL-1 through IL-25 (including, e.g., IL-2, IL-4, IL-12 and IL-18), monocyte chemoattractant protein, leukemia inhibitory factor, granulocyte-macrophage colony stimulating factor, Fas ligand, tumor necrosis factors α and β, interferons α, β, and γ, stem cell factor, flk-2/flt3 ligand. The therapeutic protein, as used herein, also comprises, without limitations, immunoglobulins IgG, IgM, IgA, IgD and IgE, chimeric immunoglobulins, humanized antibodies, single chain antibodies, T cell receptors, chimeric T cell receptors, single chain T cell receptors, class I and class II MHC molecules, as well as engineered immunoglobulins and MHC molecules. Further, the therapeutic protein, as used herein, comprises complement regulatory proteins such as membrane cofactor protein (MCP), decay accelerating factor (DAF), CR1, CF2 and CD59.

The therapeutic protein, as used herein, can also comprise any one of the receptors for the hormones, growth factors, cytokines, lymphokines, regulatory proteins and immune system proteins. The disclosure encompasses receptors for cholesterol regulation and/or lipid modulation, including the low density lipoprotein (LDL) receptor, high density lipoprotein (HDL) receptor, the very low density lipoprotein (VLDL) receptor, and scavenger receptors. The disclosure also encompasses gene products such as members of the steroid hormone receptor superfamily including glucocorticoid receptors and estrogen receptors, Vitamin D receptors and other nuclear receptors. In addition, the therapeutic protein comprises transcription factors such as jun, fos, max, mad, serum response factor (SRF), AP-1, AP2, myb, MyoD and myogenin, ETS-box containing proteins, TFE3, E2F, ATF1, ATF2, ATF3, ATF4, ZF5, NFAT, CREB, HNF-4, C/EBP, SP1, CCAAT-box binding proteins, interferon regulation factor (IRF-1), Wilms tumor protein, ETS-binding protein, STAT, GATA-box binding proteins, e.g., GATA-3, and the forkhead family of winged helix proteins.

The therapeutic protein can also comprise carbamoyl synthetase I, ornithine transcarbamylase, arginosuccinate synthetase, arginosuccinate lyase, arginase, fumarylacetacetate hydrolase, phenylalanine hydroxylase, alpha-1 antitrypsin, glucose-6-phosphatase, porphobilinogen deaminase, cystathione beta-synthase, branched chain ketoacid decarboxylase, albumin, isovaleryl-coA dehydrogenase, propionyl CoA carboxylase, methyl malonyl CoA mutase, glutaryl CoA dehydrogenase, insulin, beta-glucosidase, pyruvate carboxylate, hepatic phosphorylase, phosphorylase kinase, glycine decarboxylase, H-protein, T-protein, a cystic fibrosis transmembrane regulator (CFTR) sequence, and a dystrophin gene product. The therapeutic protein can also comprise enzymes such as may be useful in enzyme replacement therapy, which is useful in a variety of conditions resulting from deficient activity of enzyme. Non-limiting, exemplary enzyme comprises CDKL5, alpha-galactosidase, β-galactosidase, β-hexosaminidase, galactosylceramidase, arylsulfatase, β-glucocerebrosidase, glucocerebrosidase, lysosomal acid lipase, lysosomal enzyme acid sphingomyelinase, formylglycine-generating enzyme, iduronidase, acetyl-CoA:alpha-glucosaminide N-acetyltransferase, glycosaminoglycan alpha-L-iduronohydrolase, heparan N-sulfatase, N-acetyl-α-D-glucosaminidase (NAGLU), iduronate-2-sulfatase, galactosamine-6-sulfate sulfatase, N-acetylgalactosamine-6-sulfatase, glycosaminoglycan N-acetylgalactosamine 4-sulfatase, β-glucuronidase, hyaluronidase, alpha-N-acetyl neuraminidase (sialidase), ganglioside sialidase, phosphotransferase, alpha-glucosidase, alpha-D-mannosidase, beta-D-mannosidase, aspartylglucosaminidase, alpha-L-fucosidase, battenin, palmitoyl protein thioesterases, and other Batten-related proteins, or an enzymatically active fragment thereof. In one or more embodiments, the Batten-related protein comprises ceroid-lipofuscinosis neuronal protein 1, ceroid-lipofuscinosis neuronal protein 2, ceroid-lipofuscinosis neuronal protein 3, ceroid-lipofuscinosis neuronal protein 4, ceroid-lipofuscinosis neuronal protein 5, ceroid-lipofuscinosis neuronal protein 6, ceroid-lipofuscinosis neuronal protein 7, ceroid-lipofuscinosis neuronal protein 8, ceroid-lipofuscinosis neuronal protein 19, ceroid-lipofuscinosis neuronal protein 10, ceroid-lipofuscinosis neuronal protein 11, ceroid-lipofuscinosis neuronal protein 12, ceroid-lipofuscinosis neuronal protein 13 and ceroid-lipofuscinosis neuronal protein 14.

The therapeutic protein can also comprise proteins used for treatment of hemophilia, including hemophilia B (including Factor IX) and hemophilia A (including Factor VIII and its variants, such as the light chain and heavy chain of the heterodimer and the B-deleted domain; U.S. Pat. Nos. 6,200,560 and 6,221,349).

The therapeutic protein can also comprise non-naturally occurring polypeptides, such as chimeric or hybrid polypeptides having a non-naturally occurring amino acid sequence containing insertions, deletions or amino acid substitutions. For example, single-chain engineered immunoglobulins could be useful in certain immunocompromised patients. Other types of non-naturally occurring gene sequences include antisense molecules and catalytic nucleic acids, such as ribozymes, which could be used to reduce overexpression of a target.

Reduction and/or modulation of expression of a gene is particularly desirable for treatment of hyperproliferative conditions characterized by hyperproliferating cells, as are cancers and psoriasis. The therapeutic protein also comprises oncogene products such as target antigens and target polypeptides, wherein target polypeptides comprises proteins which are produced exclusively or at higher levels in hyperproliferative cells as compared to normal cells and target antigens comprises proteins encoded by oncogenes such as myb, myc, fyn, and the translocation gene bcr/abl, ras, src, P53, neu, trk and EGRF. In addition to oncogene products as target antigens, target polypeptides for anti-cancer treatments and protective regimens, the therapeutic protein also comprises variable regions of antibodies made by B cell lymphomas and variable regions of T cell receptors of T cell lymphomas which, in some embodiments, are also used as target antigens for autoimmune disease. Other tumor-associated polypeptides can be used as target polypeptides such as polypeptides which are found at higher levels in tumor cells including the polypeptide recognized by monoclonal antibody 17-1A and folate binding polypeptides.

Other suitable therapeutic proteins include those which may be useful for treating individuals suffering from autoimmune diseases and disorders by conferring a broad based protective immune response against targets that are associated with autoimmunity including cell receptors and cells which produce “self”-directed antibodies. T cell mediated autoimmune diseases include Rheumatoid arthritis (RA), multiple sclerosis (MS), Sjögren's syndrome, sarcoidosis, insulin dependent diabetes mellitus (IDDM), autoimmune thyroiditis, reactive arthritis, ankylosing spondylitis, scleroderma, polymyositis, dermatomyositis, psoriasis, vasculitis, Wegener's granulomatosis, Crohn's disease and ulcerative colitis. Each of these diseases is characterized by T cell receptors (TCRs) that bind to endogenous antigens and initiate the inflammatory cascade associated with autoimmune diseases.

The term “treating” refer to administering an agent, or carrying out a procedure, for the purposes of obtaining a therapeutic effect, including inhibiting, attenuating, reducing, preventing or altering at least one aspect or marker of a disorder, in a statistically significant manner or in a clinically significant manner. The term “treat” does not state or imply a cure for the underlying condition, rather includes: (a) preventing the disorder or a symptom of a disorder from occurring in a patient which may be predisposed to the disorder but has not yet been diagnosed as having it (e.g., including disorders that may be associated with or caused by a primary disorder; (b) inhibiting the disorder, i.e., arresting its development; (c) relieving the disorder, i.e., causing regression of the disorder; and (d) improving at least one symptom of the disorder. Treating may refer to any indicia of success in the treatment or amelioration or prevention of a disorder, including any objective or subjective parameter such as abatement; remission; diminishing of symptoms or making the disorder condition more tolerable to the patient; slowing in the rate of degeneration or decline; or making the final point of degeneration less debilitating. The treatment or amelioration of symptoms is based on one or more objective or subjective parameters; including the results of an examination by a physician. Accordingly, the term “treating” includes the administration of the compounds or agents of the present disclosure to prevent or delay, to alleviate, or to arrest or inhibit development of the symptoms or conditions associated with the disorder.

The term “vector” refers to gene therapy delivery vehicles, or carriers, that deliver therapeutic genes to cells. A vector is any vector suitable for use in gene therapy, e.g., any vector suitable for the therapeutic delivery of nucleic acid polymers (encoding a polypeptide or a variant thereof) into target cells of a patient. In some embodiments, the gene therapy vector delivers the nucleic acid encoding a fusion protein to a cell where the fusion protein is expressed and secreted from the cell. The vector may be of any type, for example it may be a plasmid vector or a minicircle DNA. Typically, the vector is a viral vector. The viral vector may, for example, be derived from an adeno-associated virus (AAV), a retrovirus, a lentivirus, a herpes simplex virus, or an adenovirus. The viral vectors also may include additional elements for increasing expression and/or stabilizing the vector such as promoters (e.g., hybrid CBA promoter (CBh) and human synapsin 1 promoter (hSyn1)), a polyadenylation signals (e.g. Bovine growth hormone polyadenylation signal (bGHpolyA)), stabilizing elements (e.g. Woodchuck Hepatitis Virus (WHP) Posttranscriptional Regulatory Element (WPRE)) and/or an SV40 intron. In one or more embodiments, a vector may comprise a polynucleotide sequence flanking by regions that promote homologous recombination at a desired site in the genome, thus providing for expression of the desired protein (See Koller and Smithies, 1989, Proc. Natl. Acad. Sci. USA, 86:8932-8935; Zijlstra et al., 1989, Nature 342:435-438; U.S. Pat. No. 6,244,113 to Zarling et al.; and U.S. Pat. No. 6,200,812 to Pati et al.).

### AAV Vectors

### AAV Vectors to Deliver Transgene

In various embodiments, the gene therapy compositions and/or methods utilize AAV vectors. Alternatively, other viral vectors or gene therapy delivery systems as described herein may be used.

AAV vectors from one of a number of clades can be utilized to deliver the transgene in vivo for transduction, and a number of examples are provided in detail in U.S. Pat. No. 7,198,951, [James Wilson patent], which is incorporated herein in its entirety. Using the genome of these AAV vectors and the manufacturing process described herein, and those available in the art, a recombinant AAV (rAAV) can be generated as a vector for delivery of one or more of the transgenes provided herein.

### I. Clades

A Glade is a group of AAV which are phylogenetically related to one another as determined using a Neighbor-Joining algorithm by a bootstrap value of at least 75% (of at least 1000 replicates) and a Poisson correction distance measurement of no more than 0.05, based on alignment of the AAV vp1 amino acid sequence.

The Neighbor-Joining algorithm has been described extensively in the literature. See, e.g., M. Nei and S. Kumar, Molecular Evolution and Phylogenetics (Oxford University Press, New York (2000)). Computer programs are available that can be used to implement this algorithm. For example, the MEGA v2.1 program implements the modified Nei-Gojobori method. Using these techniques and computer programs, and the sequence of an AAV vp1 capsid protein, one of skill in the art can readily determine whether a selected AAV is contained in one of the clades identified herein, in another Glade, or is outside these clades.

While the clades defined herein are based primarily upon naturally occurring AAV vp1 capsids, the clades are not limited to naturally occurring AAV. The clades can encompass non-naturally occurring AAV, including, without limitation, recombinant, modified or altered, chimeric, hybrid, synthetic, artificial, etc., AAV which are phylogenetically related as determined using a Neighbor-Joining algorithm at least 75% (of at least 1000 replicates) and a Poisson correction distance measurement of no more than 0.05, based on alignment of the AAV vp1 amino acid sequence.

The clades described herein include Clade A (represented by AAV1 and AAV6), Clade B (represented by AAV2) and Clade C (represented by the AAV2-AAV3 hybrid), Clade D (represented by AAV7), Clade E (represented by AAV8), and Clade F (represented by human AAV9).

Clade B (AAV2) and Clade C (the AAV2-AAV3 hybrid) are the most abundant of those found in humans (22 isolates from 12 individuals for AAV2 and 17 isolates from 8 individuals for Clade C).

### Clade A (Represented by AAV1 and AAV6)

AAV vectors can include those of Clade A, which includes AAV1 and AAV6. See, e.g., International Publication No. WO 00/28061, 18 May 2000; Rutledge et al, J Virol, 72(1):309-319 (January 1998). In addition, this Glade contains AAV described in U.S. Pat. No. 7,198,951.

### Clade B (AAV2 Clade)

In one or more embodiments, the AAV vectors include those of Clade B, including AAV2 and those described in U.S. Pat. No. 7,198,951. In one embodiment, one or more of the members of this Glade has a capsid with an amino acid identity of at least 85% identity, at least 90% identity, at least 95% identity, or at least 97% identity over the full-length of the vp1, the vp2, or the vp3 of the AAV2 capsid.

### Clade C (AAV2-AAV3 Hybrid Clade)

In one or more embodiments, the AAV vectors include those of Clade C, which is characterized by containing AAV that are hybrids of the previously published AAV2 and AAV3, and those described in U.S. Pat. No. 7,198,951. In one embodiment, one or more of the members of this Glade has a capsid with an amino acid identity of at least 85% identity, at least 90% identity, at least 95% identity, or at least 97% identity over the full-length of the vp1, the vp2, or the vp3 of the hu.4 and/or hu.2 capsid.

### Clade D (AAV7 Glade)

In one or more embodiments, the AAV vectors include those of Clade D, which includes AAV7 those described in U.S. Pat. No. 7,198,951. In one embodiment, one or more of the members of this Glade has a capsid with an amino acid identity of at least 85% identity, at least 90% identity, at least 95% identity, or at least 97% identity over the full-length of the vp1, the vp2, or the vp3 of the AAV7 capsid.

### Clade E (AAV8 Glade)

In one or more embodiments, the AAV vectors include those of Clade E, including AAV8 and those described in U.S. Pat. No. 7,198,951. In one embodiment, one or more of the members of this Glade has a capsid with an amino acid identity of at least 85% identity, at least 90% identity, at least 95% identity, or at least 97% identity over the full-length of the vp1, the vp2, or the vp3 of the AAV8 capsid. In one or more embodiments, the AAV vectors include those of Clade E as described in US Published Patent Application No. US 2003/0138772 A1 (Jul. 24 2003).

### Clade F (AAV 9 Clade)

In one or more embodiments, the AAV vectors include those of Clade F, including AAV9 and those described in U.S. Pat. No. 7,198,951. In one embodiment, one or more of the members of this Glade has a capsid with an amino acid identity of at least 85% identity, at least 90% identity, at least 95% identity, or at least 97% identity over the full-length of the vp1, the vp2, or the vp3 of the AAV9 capsid.

The AAV clades are useful for a variety of purposes, including providing ready collections of related AAV for generating viral vectors, and for generating targeting molecules. These clades may also be used as tools for a variety of purposes that will be readily apparent to one of skill in the art.

### Transgene

The transgene is a nucleic acid sequence, heterologous to the vector sequences flanking the transgene, which encodes a polypeptide, protein, or other product, of interest. The nucleic acid coding sequence can be operatively linked to regulatory components in a manner which permits transgene transcription, translation, and/or expression in a host cell.

The transgene may be used to correct or ameliorate gene deficiencies, which may include deficiencies in which normal genes are expressed at less than normal levels or deficiencies in which the functional gene product is not expressed. Alternatively, the transgene may provide a product to a cell which is not natively expressed in the cell type or in the host. A preferred type of transgene sequence encodes a therapeutic protein or polypeptide which is expressed in a host cell. In one or more embodiments, multiple transgenes are used. In certain situations, a different transgene may be used to encode each subunit of a protein, or to encode different peptides or proteins. This is desirable when the size of the DNA encoding the protein subunit is large, e.g., for an immunoglobulin, the platelet-derived growth factor, or a dystrophin protein. In order for the cell to produce the multi-subunit protein, a cell is infected with the recombinant virus containing each of the different subunits. Alternatively, different subunits of a protein may be encoded by the same transgene. In this case, a single transgene includes the DNA encoding each of the subunits, with the DNA for each subunit separated by an internal ribozyme entry site (IRES). This is desirable when the size of the DNA encoding each of the subunits is small, e.g., the total size of the DNA encoding the subunits and the IRES is less than five kilobases. As an alternative to an IRES, the DNA may be separated by sequences encoding a 2A peptide, which self-cleaves in a post-translational event. See, e.g., M. L. Donnelly, et al, J. Gen. Virol., 78(Pt 1):13-21 (January 1997); Furler, S., et al, Gene Ther., 8(11):864-873 (June 2001); Klump H., et al., Gene Ther., 8(10):811-817 (May 2001). This 2A peptide is significantly smaller than an IRES, making it well suited for use when space is a limiting factor. More often, when the transgene is large, consists of multi-subunits, or two transgenes are co-delivered. In some embodiments, rAAV carrying the desired transgene(s) or subunits can be co-administered to allow them to concatamerize in vivo to form a single vector genome. In such an embodiment, a first AAV may carry an expression cassette which expresses a single transgene and a second AAV may carry an expression cassette which expresses a different transgene for co-expression in the host cell. However, the selected transgene may encode any biologically active product or other product, e.g., a product desirable for study.

### Therapeutic Transgenes

Useful therapeutic products encoded by the transgene include hormones and/or growth and/or differentiation factors including, without limitation, insulin, glucagon, growth hormone (GH), parathyroid hormone (PTH), growth hormone releasing factor (GRF), follicle stimulating hormone (FSH), luteinizing hormone (LH), human chorionic gonadotropin (hCG), vascular endothelial growth factor (VEGF), angiopoietins, angiostatin, granulocyte colony stimulating factor (GCSF), erythropoietin (EPO), connective tissue growth factor (CTGF), basic fibroblast growth factor (bFGF), acidic fibroblast growth factor (aFGF), epidermal growth factor (EGF), platelet-derived growth factor (PDGF), insulin growth factors I and II (IGF-I and IGF-II), any one of the transforming growth factor a superfamily, including TGFα, activins, inhibins, or any of the bone morphogenic proteins (BMP) BMPs 1-15, any one of the heregluin/neuregulin/ARIA/neu differentiation factor (NDF) family of growth factors, nerve growth factor (NGF), brain-derived neurotrophic factor (BDNF), neurotrophins NT-3 and NT-4/5, ciliary neurotrophic factor (CNTF), glial cell line derived neurotrophic factor (GDNF), neurturin, agrin, any one of the family of semaphorins/collapsins, netrin-1 and netrin-2, hepatocyte growth factor (HGF), ephrins, noggin, sonic hedgehog and tyrosine hydroxylase.

Other useful transgene products include proteins that regulate the immune system including, without limitation, cytokines and lymphokines such as thrombopoietin (TPO), interleukins (IL) IL-1 through IL-25 (including, e.g., IL-2, IL-4, IL-12 and IL-18), monocyte chemoattractant protein, leukemia inhibitory factor, granulocyte-macrophage colony stimulating factor, Fas ligand, tumor necrosis factors α and β, interferons α, β, and γ, stem cell factor, flk-2/flt3 ligand. Gene products produced by the immune system are also useful in the invention. These include, without limitations, immunoglobulins IgG, IgM, IgA, IgD and IgE, chimeric immunoglobulins, humanized antibodies, single chain antibodies, T cell receptors, chimeric T cell receptors, single chain T cell receptors, class I and class II MHC molecules, as well as engineered immunoglobulins and MHC molecules. Useful gene products also include complement regulatory proteins such as complement regulatory proteins, membrane cofactor protein (MCP), decay accelerating factor (DAF), CR1, CF2 and CD59.

Still other useful gene products include any one of the receptors for the hormones, growth factors, cytokines, lymphokines, regulatory proteins and immune system proteins. The invention encompasses receptors for cholesterol regulation and/or lipid modulation, including the low density lipoprotein (LDL) receptor, high density lipoprotein (HDL) receptor, the very low density lipoprotein (VLDL) receptor, and scavenger receptors. The invention also encompasses gene products such as members of the steroid hormone receptor superfamily including glucocorticoid receptors and estrogen receptors, Vitamin D receptors and other nuclear receptors. In addition, useful gene products include transcription factors such as jun, fos, max, mad, serum response factor (SRF), AP-1, AP2, myb, MyoD and myogenin, ETS-box containing proteins, TFE3, E2F, ATF1, ATF2, ATF3, ATF4, ZFS, NFAT, CREB, HNF-4, C/EBP, SP1, CCAAT-box binding proteins, interferon regulation factor (IRF-1), Wilms tumor protein, ETS-binding protein, STAT, GATA-box binding proteins, e.g., GATA-3, and the forkhead family of winged helix proteins.

Other useful gene products include, carbamoyl synthetase I, ornithine transcarbamylase, arginosuccinate synthetase, arginosuccinate lyase, arginase, fumarylacetacetate hydrolase, phenylalanine hydroxylase, alpha-1 antitrypsin, glucose-6-phosphatase, porphobilinogen deaminase, cystathione beta-synthase, branched chain ketoacid decarboxylase, albumin, isovaleryl-coA dehydrogenase, propionyl CoA carboxylase, methyl malonyl CoA mutase, glutaryl CoA dehydrogenase, insulin, beta-glucosidase, pyruvate carboxylate, hepatic phosphorylase, phosphorylase kinase, glycine decarboxylase, H-protein, T-protein, a cystic fibrosis transmembrane regulator (CFTR) sequence, and a dystrophin gene product. Still other useful gene products include enzymes such as may be useful in enzyme replacement therapy, which is useful in a variety of conditions resulting from deficient activity of enzyme. For example, enzymes that contain mannose-6-phosphate may be utilized in therapies for lysosomal storage diseases (e.g., a suitable gene includes that encoding (3-glucuronidase (GUSB)).

Still other useful gene products include those used for treatment of hemophilia, including hemophilia B (including Factor IX) and hemophilia A (including Factor VIII and its variants, such as the light chain and heavy chain of the heterodimer and the B-deleted domain; U.S. Pat. Nos. 6,200,560 and 6,221,349). The Factor VIII gene codes for 2351 amino acids and the protein has six domains, designated from the amino to the terminal carboxy terminus as A1-A2-B-A3-C1-C2 [Wood et al, Nature, 312:330 (1984); Vehar et al., Nature 312:337 (1984); and Toole et al, Nature, 342:337 (1984)]. Human Factor VIII is processed within the cell to yield a heterodimer primarily comprising a heavy chain containing the A1, A2 and B domains and a light chain containing the A3, C1 and C2 domains. Both the single chain polypeptide and the heterodimer circulate in the plasma as inactive precursors, until activated by thrombin cleavage between the A2 and B domains, which releases the B domain and results in a heavy chain consisting of the A1 and A2 domains. The B domain is deleted in the activated procoagulant form of the protein. Additionally, in the native protein, two polypeptide chains (“a” and “b”), flanking the B domain, are bound to a divalent calcium cation.

In some embodiments, the minigene comprises the first 57 base pairs of the Factor VIII heavy chain which encodes the 10 amino acid signal sequence, as well as the human growth hormone (hGH) polyadenylation sequence. In alternative embodiments, the minigene further comprises the A1 and A2 domains, as well as 5 amino acids from the N-terminus of the B domain, and/or 85 amino acids of the C-terminus of the B domain, as well as the A3, C1 and C2 domains. In yet other embodiments, the nucleic acids encoding Factor VIII heavy chain and light chain are provided in a single minigene separated by 42 nucleic acids coding for 14 amino acids of the B domain [U.S. Pat. No. 6,200,560].

As used herein, a therapeutically effective amount of an AAV vector is an amount of AAV vector that produces sufficient amounts of Factor VIII to decrease the time it takes for a subject's blood to clot. Generally, severe hemophiliacs having less than 1% of normal levels of Factor VIII have a whole blood clotting time of greater than 60 minutes as compared to approximately 10 minutes for non-hemophiliacs.

The present invention is not limited to any specific Factor VIII sequence. Many natural and recombinant forms of Factor VIII have been isolated and generated. Examples of naturally occurring and recombinant forms of Factor VII can be found in the patent and scientific literature including, U.S. Pat. Nos. 5,563,045, 5,451,521, 5,422,260, 5,004,803, 4,757,006, 5,661,008, 5,789,203, 5,681,746, 5,595,886, 5,045,455, 5,668,108, 5,633,150, 5,693,499, 5,587,310, 5,171,844, 5,149,637, 5,112,950, 4,886,876; International Patent Publication Nos. WO 94/11503, WO 87/07144, WO 92/16557, WO 91/09122, WO 97/03195, WO 96/21035, and WO 91/07490; European Patent Application Nos. EP 0 672 138, EP 0 270 618, EP 0 182 448, EP 0 162 067, EP 0 786 474, EP 0 533 862, EP 0 506 757, EP 0 874 057,EP 0 795 021, EP 0 670 332, EP 0 500 734, EP 0 232 112, and EP 0 160 457; Sanberg et al., XXth Int. Congress of the World Fed. Of Hemophilia (1992), and Lind et al., Eur. J Biochem., 232:19 (1995).

Nucleic acids sequences coding for the above-described Factor VIII can be obtained using recombinant methods or by deriving the sequence from a vector known to include the same. Furthermore, the desired sequence can be isolated directly from cells and tissues containing the same, using standard techniques, such as phenol extraction and PCR of cDNA or genomic DNA [See, e.g., Sambrook et al]. Nucleotide sequences can also be produced synthetically, rather than cloned. The complete sequence can be assembled from overlapping oligonucleotides prepared by standard methods and assembled into a complete coding sequence [See, e.g., Edge, Nature 292:757 (1981); Nambari et al, Science, 223:1299 (1984); and Jay et al, J. Biol. Chem. 259:6311 (1984).

Furthermore, the invention is not limited to human Factor VIII. Indeed, it is intended that the present invention encompass Factor VIII from animals other than humans, including but not limited to companion animals (e.g., canine, felines, and equines), livestock (e.g., bovines, caprines and ovines), laboratory animals, marine mammals, large cats, etc.

The AAV vectors may contain a nucleic acid coding for fragments of Factor VIII which is itself not biologically active, yet when administered into the subject improves or restores the blood clotting time. For example, as discussed above, the Factor VIII protein comprises two polypeptide chains: a heavy chain and a light chain separated by a B-domain which is cleaved during processing. As demonstrated by the present invention, co-tranducing recipient cells with the Factor VIII heavy and light chains leads to the expression of biologically active Factor VIII. Because most hemophiliacs contain a mutation or deletion in only one of the chains (e.g., heavy or light chain), it may be possible to administer only the chain defective in the patient to supply the other chain.

Other useful gene products include non-naturally occurring polypeptides, such as chimeric or hybrid polypeptides having a non-naturally occurring amino acid sequence containing insertions, deletions or amino acid substitutions. For example, single-chain engineered immunoglobulins could be useful in certain immunocompromised patients. Other types of non-naturally occurring gene sequences include antisense molecules and catalytic nucleic acids, such as ribozymes, which could be used to reduce overexpression of a target.

Reduction and/or modulation of expression of a gene is particularly desirable for treatment of hyperproliferative conditions characterized by hyperproliferating cells, as are cancers and psoriasis. Target polypeptides include those polypeptides which are produced exclusively or at higher levels in hyperproliferative cells as compared to normal cells. Target antigens include polypeptides encoded by oncogenes such as myb, myc, fyn, and the translocation gene bcr/abl, ras, src, P53, neu, trk and EGRF. In addition to oncogene products as target antigens, target polypeptides for anti-cancer treatments and protective regimens include variable regions of antibodies made by B cell lymphomas and variable regions of T cell receptors of T cell lymphomas which, in some embodiments, are also used as target antigens for autoimmune disease. Other tumor-associated polypeptides can be used as target polypeptides such as polypeptides which are found at higher levels in tumor cells including the polypeptide recognized by monoclonal antibody 17-1A and folate binding polypeptides.

Other suitable therapeutic polypeptides and proteins include those which may be useful for treating individuals suffering from autoimmune diseases and disorders by conferring a broad based protective immune response against targets that are associated with autoimmunity including cell receptors and cells which produce “self”-directed antibodies. T cell mediated autoimmune diseases include Rheumatoid arthritis (RA), multiple sclerosis (MS), Sjögren's syndrome, sarcoidosis, insulin dependent diabetes mellitus (IDDM), autoimmune thyroiditis, reactive arthritis, ankylosing spondylitis, scleroderma, polymyositis, dermatomyositis, psoriasis, vasculitis, Wegener's granulomatosis, Crohn's disease and ulcerative colitis. Each of these diseases is characterized by T cell receptors (TCRs) that bind to endogenous antigens and initiate the inflammatory cascade associated with autoimmune diseases.

### Exosome and Vesicle Targeting Protein

Vesicles comprises of exosomes and extracellular vesicles. Exosomes are small membrane-bound vesicles 30-130 nm, which either bud from the cytoplasm into multi-vesicular bodies (MVBs) within cells or directly into the extracellular space from the plasma membrane. MVBs are part of the endocytic pathway and can release their cargo of exosomes into the extracellular space after fusing with the plasma membrane.

A number of proteins are known to be associated with exosomes; that is they are incorporated into the exosome as it is formed. The preferred proteins for use in accordance with the present invention are those which are transmembrane proteins or membrane associated proteins. Examples include but are not limited to Lamp-1, Lamp-2, CD13, CD86, Flotillin, Syntaxin-3, CD2, CD36, CD40, CD40L, CD41a, CD44, CD45, ICAM-1, Integrin alpha4, LiCAM, LFA-1, Mac-1 alpha and beta, Vti-1A and B, CD3 epsilon and zeta, CD9, CD18, CD37, CD53, CD63, CD81, CD82, CXCR4, FcR, GluR2/3, HLA-DM (MHC II), immunoglobulins, MHC-I or MHC-II components, TCR beta and tetraspanins. In particularly preferred embodiments of the present invention, the transmembrane protein is selected from Lamp-1, Lamp-2, CD13, CD86, Flotillin, Syntaxin-3.

### Nef Protein and Variants

Nef is a protein expressed by primate lentiviruses, such as HIV and SIV. Nef is known to be secreted in association with exosomes and has been also shown to be present on the surface of HIV-infected cells.

Nef protein can be fused to the transgene. In some embodiments the Nef protein is a fragment thereof, which can be fragment of amino acid 1-70, 1-75, 1-80, 1-85, 1-90, 1-95, 1-100, 1-105, 1-110, 1-115, 1-120, 1-125, 1-130, 1-135, 1-140, 1-145, 1-150, 1-155, 1-160, 1-165, 1-170, 1-175, 1-180, 1-185, 1-190, 1-195, 1-200, or 1-205. Preferably the fragment is from 1-70 aa. The Nef protein can be a mutated version including G3C, V153L, E177G, or a combination thereof, and preferably includes all three point mutations, which is referred to as Nef^{mut}. In some embodiments, a polynucleotide comprises a nucleotide sequence encoding Nef protein or a variant thereof.

### Membrane Associated-ED Protein and Variants

One aspect of the disclosure describes membrane associated-ED protein and variants thereof. In some embodiments, the membrane associated-ED protein comprises one or more of a signal domain, a curvature sensing domain, and an Effector domain (ED domain).

The MARCKS (myristoylated alanine-rich C kinase substrate) family of proteins is known to be associated with exosomes. Accordingly, in some embodiments, the MARCKS proteins comprises one or more of a signal domain, a curvature sensing domain, and an Effector domain (ED domain), wherein the signal domain comprises a myristoylation signal domain.

MARCKS-like protein 1 or MARCKSL1 (myristoylated alanine-rich C kinase substrate) family of protein is known to be associated with macrophages. Similar to MARCKS protein, in some embodiments, MARCKSL1 also comprises one or more of a signal domain, a curvature sensing domain, and an Effector domain (ED domain), wherein the signal domain comprises a myristoylation signal domain.

BASP1 family of proteins is also known to be associated with exosomes. The BASP1 proteins are mainly found in neuronal tissues. Similar to MARCKS proteins, in some embodiments, BASP1 proteins also comprises one or more of a signal domain, a curvature sensing domain, and an Effector domain (ED domain), wherein the signal domain comprises a myristoylation signal domain.

One or more embodiments comprise membrane associated-ED protein, wherein the membrane associated-ED protein comprises a signal domain and ED domain. In some embodiments, the signal domain is on N-terminus of the membrane associated-ED protein. In some embodiments, the signal domain is directly linked to the ED domain. In some embodiments, the signal domain is linked to the ED domain by a first linker peptide. The first linker peptide has an amino sequence according to SEQ ID NO: 143, SEQ ID NO: 144, SEQ ID NO: 145, SEQ ID NO: 146, SEQ ID NO: 147, SEQ ID NO: 148, SEQ ID NO: 149, SEQ ID NO: 150, SEQ ID NO: 151, SEQ ID NO: 152 or SEQ ID NO: 153.

The disclosure provides the variants of MARCKS, MARCKSL1 and BASP1 proteins. Accordingly, in one or more embodiments, the membrane associated-ED protein comprises MyrED protein, wherein the MyrED protein comprises myristoylation signal domain and ED domain.

In some embodiments, the membrane associated-ED protein comprises BiPED protein, the BiPED protein comprises a BiP secretion signal domain and ED domain. In some embodiments, the BiP secretion signal domain comprises at least 50%, at least 55%, at least 60%, at least 65%, at least 70%, at least 75%, at least 80%, at least 85%, at least 90%, or at least 95% amino acid sequence similarity to SEQ ID NO: 155, SEQ ID NO: 156, SEQ ID NO: 157, SEQ ID NO: 158, SEQ ID NO: 159 or SEQ ID NO: 165.

The signal domain may comprise at least 50%, at least 55%, at least 60%, at least 65%, at least 70%, at least 75%, at least 80%, at least 85%, at least 90%, or at least 95% sequence similarity to any one of amino acid sequences of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 155, SEQ ID NO: 156, SEQ ID NO: 157, SEQ ID NO: 158, SEQ ID NO: 159 or SEQ ID NO: 165. The disclosure also provides a polynucleotide comprising a nucleotide sequence such that resulting polypeptide has at least 50%, at least 55%, at least 60%, at least 65%, at least 70%, at least 75%, at least 80%, at least 85%, at least 90%, or at least 95% sequence similarity to SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 155, SEQ ID NO: 156, SEQ ID NO: 157, SEQ ID NO: 158, SEQ ID NO: 159 or SEQ ID NO: 165. Alternatively, the signal domain may comprise any one of amino acid sequences of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 155, SEQ ID NO: 156, SEQ ID NO: 157, SEQ ID NO: 158, SEQ ID NO: 159 or SEQ ID NO: 165. The disclosure also provides a polynucleotide comprising a nucleotide sequence such that resulting polypeptide sequence is any one of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 155, SEQ ID NO: 156, SEQ ID NO: 157, SEQ ID NO: 158, SEQ ID NO: 159 or SEQ ID NO: 165. In some embodiments, the signal domain comprises myristoylation signal domain.

In some embodiments, the signal domain comprises a natively glycosylated protein leader sequence. In some embodiments, the natively glycosylated protein leader sequence is derived from neprilysin, bassoon, human hepatitis B virus preS, duck hepatitis B virus preS, Lassa envelope, Machupo envelope or combinations thereof.

Farnesylation is a type of C-terminal lipidation with a similar strength of membrane binding as myristoylation. The primary sequence of KRAS4b with a lysine rich region near the farnesylated cysteine reminiscent of the Marcks ED domain functions in a similar manner to MARCKS protein. KRAS4b interacts with calmodulin in a calcium dependent manner that sequesters it from binding to the membrane. Accordingly, in some embodiments, the signal domain comprises KRAS4b or a variant thereof. In some embodiments, KRAS4b or a variant thereof comprises sequence at least 50%, at least 55%, at least 60%, at least 65%, at least 70%, at least 75%, at least 80%, at least 85%, at least 90%, or at least 95% sequence similarity to KRAS4b. In one or more embodiments, the KRAS4b sequence comprises CVIM (SEQ ID NO: 165). A series of sequences (MYRED88-93) merging KRAS4b and the minimized-ED domain from Marcks were made and tested (SEQ ID NOS: 118-123 and 140-142).

Glypiation is the addition of the strong membrane anchor, glycosylphosphatidylinositol (GPI) to the protein. Accordingly, in some embodiments, the signal domain comprises a GPI anchoring signal peptide. In some embodiments, the GPI anchoring signal peptide comprises a GPI anchoring signal from COBL9_ARATH. COBL9_ARATH is COBRA-like protein 9 from Aradopsis thaliana and has been shown to play a role in the orientation of microfibrils and cellulose crystallinity.

The ED domain may comprise at least 50%, at least 55%, at least 60%, at least 65%, at least 70%, at least 75%, at least 80%, at least 85%, at least 90%, or at least 95% sequence similarity to SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, or SEQ ID NO: 30. The disclosure also provides a polynucleotide comprising a nucleotide sequence such that resulting polypeptide has at least 50%, at least 55%, at least 60%, at least 65%, at least 70%, at least 75%, at least 80%, at least 85%, at least 90%, or at least 95% sequence similarity to SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, or SEQ ID NO: 30.

Alternatively, the ED domain comprises any one of amino acid sequences of SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, or SEQ ID NO: 30. The disclosure also provides a polynucleotide comprising a nucleotide sequence such that resulting polypeptide sequence is any one of SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, and SEQ ID NO: 30.

The membrane associated-ED protein, amino acid sequence comprising at least 50%, at least 55%, at least 60%, at least 65%, at least 70%, at least 75%, at least 80%, at least 85%, at least 90%, or at least 95% sequence similarity to SEQ ID NO:31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 121, SEQ ID NO: 122, SEQ ID NO: 123, SEQ ID NO: 124, SEQ ID NO: 125, SEQ ID NO: 126, SEQ ID NO: 127, SEQ ID NO: 128, SEQ ID NO: 129, SEQ ID NO: 130, SEQ ID NO: 131, SEQ ID NO: 132, SEQ ID NO: 133, SEQ ID NO: 134, SEQ ID NO: 135, SEQ ID NO: 136, SEQ ID NO: 137, SEQ ID NO: 138, SEQ ID NO: 139, SEQ ID NO: 140, SEQ ID NO: 141 or SEQ ID NO: 142. Alternatively, the disclosure provides the membrane associated-ED amino acid sequence comprising SEQ ID NO:31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 121, SEQ ID NO: 122, SEQ ID NO: 123, SEQ ID NO: 124, SEQ ID NO: 125, SEQ ID NO: 126, SEQ ID NO: 127, SEQ ID NO: 128, SEQ ID NO: 129, SEQ ID NO: 130, SEQ ID NO: 131, SEQ ID NO: 132, SEQ ID NO: 133, SEQ ID NO: 134, SEQ ID NO: 135, SEQ ID NO: 136, SEQ ID NO: 137, SEQ ID NO: 138, SEQ ID NO: 139, SEQ ID NO: 140, SEQ ID NO: 141 or SEQ ID NO: 142. In one or more embodiments, the amino acid sequence of the membrane associated-ED protein is not SEQ ID NO: 38.

The disclosure also provides a polynucleotide comprising a nucleotide sequence such that resulting polypeptide has at least 50%, at least 55%, at least 60%, at least 65%, at least 70%, at least 75%, at least 80%, at least 85%, at least 90%, or at least 95% sequence similarity to SEQ ID NO:31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 121, SEQ ID NO: 122, SEQ ID NO: 123, SEQ ID NO: 124, SEQ ID NO: 125, SEQ ID NO: 126, SEQ ID NO: 127, SEQ ID NO: 128, SEQ ID NO: 129, SEQ ID NO: 130, SEQ ID NO: 131, SEQ ID NO: 132, SEQ ID NO: 133, SEQ ID NO: 134, SEQ ID NO: 135, SEQ ID NO: 136, SEQ ID NO: 137, SEQ ID NO: 138, SEQ ID NO: 139, SEQ ID NO: 140, SEQ ID NO: 141 or SEQ ID NO: 142. Alternatively, the disclosure provides a polynucleotide comprising a nucleotide sequence encoding SEQ ID NO:31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 121, SEQ ID NO: 122, SEQ ID NO: 123, SEQ ID NO: 124, SEQ ID NO: 125, SEQ ID NO: 126, SEQ ID NO: 127, SEQ ID NO: 128, SEQ ID NO: 129, SEQ ID NO: 130, SEQ ID NO: 131, SEQ ID NO: 132, SEQ ID NO: 133, SEQ ID NO: 134, SEQ ID NO: 135, SEQ ID NO: 136, SEQ ID NO: 137, SEQ ID NO: 138, SEQ ID NO: 139, SEQ ID NO: 140, SEQ ID NO: 141 or SEQ ID NO: 142. In one or more embodiments, the nucleotide sequence does not encode the membrane associated-ED of SEQ ID NO: 38.

### Fusion Protein

One aspect of the present disclosure includes a fusion protein comprising a vesicle targeting protein and a protein of interest. In one or more embodiments, the vesicle targeting protein targets one or more of exosomes and extracellular vesicles.

In various embodiments, the vesicle targeting protein of the fusion protein comprises a lipid conjugating domain and an effector domain (ED). In some embodiments, the lipid conjugating domain is directly linked the effector domain (ED). In some embodiments, the lipid conjugating domain is linked to the effector domain (ED) by a second linker peptide. In one or more embodiments, the second linker peptide has an amino acid sequence comprising SEQ ID NO: 143, SEQ ID NO: 144, SEQ ID NO: 145, SEQ ID NO: 146, SEQ ID NO: 147, SEQ ID NO: 148, SEQ ID NO: 149, SEQ ID NO: 150, SEQ ID NO: 151, SEQ ID NO: 152 or SEQ ID NO: 153.

In one or more embodiments of the fusion protein, the vesicle targeting protein comprises membrane associated-ED protein. In some embodiments, the membrane associated-ED protein comprises a signal domain and an ED domain. In one or more embodiments, the protein of interest is present on C- or N-terminus of the membrane associated-ED protein. In one or more embodiments, the protein of interest is on the C-terminus of the signal domain. In one or more embodiments, the protein of interest is present on the C- or N-terminus of the ED domain. In some embodiments, the membrane associated-ED protein comprises MyrED protein. In one or more embodiments the MyrED protein comprises a myristoylation signal domain and an ED domain. In some embodiments, the membrane associated-ED protein comprises BiPED protein. In one or more embodiments the BiPED protein comprises a BiP secretion signal domain and an ED domain.

In one or more embodiments of the fusion protein, the signal domain comprises one or more of a lipid conjugating domain, e.g. myristoylation signal domain, a palmitoylation signal domain, a prenylation signal domain, a glycosylphosphatidylinositol anchor protein and a BiP secretion signal domain. In some embodiments, the lipid conjugating domain is the myristoylation signal domain.

In one or more embodiments of the fusion protein, the signal domain has at least 50%, at least 60%, at least 70%, at least 80%, at least 90%, at least 95% or 100% sequence similarity to the myristoylation signal domain of MARCKS, MARCKSL1 or BASP1 families of proteins. In one or more embodiments of the fusion protein, a polynucleotide encoding signal domain comprises a nucleotide sequence such that resulting polypeptide has at least 50%, at least 55%, at least 60%, at least 65%, at least 70%, at least 75%, at least 80%, at least 85%, at least 90%, at least 95% or 100% sequence similarity to the myristoylation signal domain of MARCKS, MARCKSL1 or BASP1 families of proteins.

In some embodiments, the signal domain has at least 50%, at least 60%, at least 70%, at least 80%, at least 90%, at least 95% or 100% sequence similarity to KRAS4b. In one or more embodiments of the fusion protein, a polynucleotide encoding signal domain comprises a nucleotide sequence such that resulting polypeptide has at least 50%, at least 55%, at least 60%, at least 65%, at least 70%, at least 75%, at least 80%, at least 85%, at least 90%, at least 95% or 100% sequence similarity to the KRAS4b. In one or more embodiments, the signal domain comprises the sequence CVIM (SEQ ID NO: 165).

In some embodiments, the signal domain has at least 50%, at least 60%, at least 70%, at least 80%, at least 90%, at least 95% or 100% sequence similarity to the GPI anchoring signal peptide. In one or more embodiments of the fusion protein, a polynucleotide encoding signal domain comprises a nucleotide sequence such that resulting polypeptide has at least 50%, at least 55%, at least 60%, at least 65%, at least 70%, at least 75%, at least 80%, at least 85%, at least 90%, at least 95% or 100% sequence similarity to the GPI anchoring signal peptide.

In one of more embodiment of the fusion protein, the signal domain comprises at least 50%, at least 55%, at least 60%, at least 65%, at least 70%, at least 75%, at least 80%, at least 85%, at least 90%, or at least 95% sequence similarity to any one of amino acid sequences of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 155, SEQ ID NO: 156, SEQ ID NO: 157, SEQ ID NO: 158, SEQ ID NO: 159 or SEQ ID NO: 165. In one or more embodiments of the fusion protein, a polynucleotide encoding signal domain comprises a nucleotide sequence such that resulting polypeptide has at least 50%, at least 55%, at least 60%, at least 65%, at least 70%, at least 75%, at least 80%, at least 85%, at least 90%, or at least 95% sequence similarity to SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 155, SEQ ID NO: 156, SEQ ID NO: 157, SEQ ID NO: 158, SEQ ID NO: 159 or SEQ ID NO: 165.

In one of more embodiment of the fusion protein, the signal domain comprises any one of amino acid sequences of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 155, SEQ ID NO: 156, SEQ ID NO: 157, SEQ ID NO: 158, SEQ ID NO: 159 or SEQ ID NO: 165. In one or more embodiments of the fusion protein, a polynucleotide encoding signal domain comprises a nucleotide sequence such that resulting polypeptide sequence is any one of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 155, SEQ ID NO: 156, SEQ ID NO: 157, SEQ ID NO: 158, SEQ ID NO: 159 or SEQ ID NO: 165.

In one of more embodiment of the fusion protein, the ED domain comprises at least 50%, at least 55%, at least 60%, at least 65%, at least 70%, at least 75%, at least 80%, at least 85%, at least 90%, or at least 95% sequence similarity to SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, or SEQ ID NO: 30. In one or more embodiments of the fusion protein, a polynucleotide encoding ED domain comprises a nucleotide sequence such that resulting polypeptide has at least 50%, at least 55%, at least 60%, at least 65%, at least 70%, at least 75%, at least 80%, at least 85%, at least 90%, or at least 95% sequence similarity to SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, or SEQ ID NO: 30.

In one of more embodiment of the fusion protein, the ED domain comprises any one of amino acid sequences of SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, or SEQ ID NO: 30. In one or more embodiments of the fusion protein, a polynucleotide encoding ED domain comprises a nucleotide sequence such that resulting polypeptide sequence is any one of SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, and SEQ ID NO: 30.

In one or more embodiments of the fusion protein, the membrane associated-ED protein amino acid sequence comprises at least 50%, at least 55%, at least 60%, at least 65%, at least 70%, at least 75%, at least 80%, at least 85%, at least 90%, or at least 95% sequence similarity to SEQ ID NO:31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 121, SEQ ID NO: 122, SEQ ID NO: 123, SEQ ID NO: 124, SEQ ID NO: 125, SEQ ID NO: 126, SEQ ID NO: 127, SEQ ID NO: 128, SEQ ID NO: 129, SEQ ID NO: 130, SEQ ID NO: 131, SEQ ID NO: 132, SEQ ID NO: 133, SEQ ID NO: 134, SEQ ID NO: 135, SEQ ID NO: 136, SEQ ID NO: 137, SEQ ID NO: 138, SEQ ID NO: 139, SEQ ID NO: 140, SEQ ID NO: 141 or SEQ ID NO: 142. In one or more embodiments of the fusion protein, a polynucleotide encoding membrane associated-ED protein comprises a nucleotide sequence such that resulting polypeptide has at least 50%, at least 55%, at least 60%, at least 65%, at least 70%, at least 75%, at least 80%, at least 85%, at least 90%, or at least 95% sequence similarity to SEQ ID NO:31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 121, SEQ ID NO: 122, SEQ ID NO: 123, SEQ ID NO: 124, SEQ ID NO: 125, SEQ ID NO: 126, SEQ ID NO: 127, SEQ ID NO: 128, SEQ ID NO: 129, SEQ ID NO: 130, SEQ ID NO: 131, SEQ ID NO: 132, SEQ ID NO: 133, SEQ ID NO: 134, SEQ ID NO: 135, SEQ ID NO: 136, SEQ ID NO: 137, SEQ ID NO: 138, SEQ ID NO: 139, SEQ ID NO: 140, SEQ ID NO: 141 or SEQ ID NO: 142.

In one or more embodiments of the fusion protein, the membrane associated-ED protein amino acid sequence comprises SEQ ID NO:31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 121, SEQ ID NO: 122, SEQ ID NO: 123, SEQ ID NO: 124, SEQ ID NO: 125, SEQ ID NO: 126, SEQ ID NO: 127, SEQ ID NO: 128, SEQ ID NO: 129, SEQ ID NO: 130, SEQ ID NO: 131, SEQ ID NO: 132, SEQ ID NO: 133, SEQ ID NO: 134, SEQ ID NO: 135, SEQ ID NO: 136, SEQ ID NO: 137, SEQ ID NO: 138, SEQ ID NO: 139, SEQ ID NO: 140, SEQ ID NO: 141 or SEQ ID NO: 142. In one or more embodiments of the fusion protein, a polynucleotide encoding membrane associated-ED protein comprises a nucleotide sequence such that resulting polypeptide is any one of SEQ ID NO:31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 121, SEQ ID NO: 122, SEQ ID NO: 123, SEQ ID NO: 124, SEQ ID NO: 125, SEQ ID NO: 126, SEQ ID NO: 127, SEQ ID NO: 128, SEQ ID NO: 129, SEQ ID NO: 130, SEQ ID NO: 131, SEQ ID NO: 132, SEQ ID NO: 133, SEQ ID NO: 134, SEQ ID NO: 135, SEQ ID NO: 136, SEQ ID NO: 137, SEQ ID NO: 138, SEQ ID NO: 139, SEQ ID NO: 140, SEQ ID NO: 141 or SEQ ID NO: 142.

In one or more embodiments of the fusion protein, the vesicle targeting protein comprises Nef protein, wherein the vesicle is exosome or extracellular vesicle. In one or more embodiments, the protein of interest is present on the C or N-terminus of the Nef protein. Nef protein can be fused to the transgene. In one or more embodiments, the Nef protein is a fragment thereof, which can be fragment of amino acid 1-70, 1-75, 1-80, 1-85, 1-90, 1-95, 1-100, 1-105, 1-110, 1-115, 1-120, 1-125, 1-130, 1-135, 1-140, 1-145, 1-150, 1-155, 1-160, 1-165, 1-170, 1-175, 1-180, 1-185, 1-190, 1-195, 1-200, or 1-205. Preferably the fragment is from 1-70 aa. The Nef protein can be a mutated version including G3C, V153L, E177G, or a combination thereof, and preferably includes all three-point mutations, which is referred to as Nef^{mut}.

In one or more embodiments of the fusion protein, the protein of interest comprises a therapeutic protein.

In one or more embodiments, the therapeutic protein comprises hormones and growth and differentiation factors such as insulin, glucagon, growth hormone (GH), parathyroid hormone (PTH), growth hormone releasing factor (GRF), follicle stimulating hormone (FSH), luteinizing hormone (LH), human chorionic gonadotropin (hCG), vascular endothelial growth factor (VEGF), angiopoietins, angiostatin, granulocyte colony stimulating factor (GCSF), erythropoietin (EPO), connective tissue growth factor (CTGF), basic fibroblast growth factor (bFGF), acidic fibroblast growth factor (aFGF), epidermal growth factor (EGF), platelet-derived growth factor (PDGF), insulin growth factors I and II (IGF-I and IGF-II), any one of the transforming growth factor a superfamily, including TGFα, activins, inhibins, or any of the bone morphogenic proteins (BMP) BMPs 1-15, any one of the heregluin/neuregulin/ARIA/neu differentiation factor (NDF) family of growth factors, nerve growth factor (NGF), brain-derived neurotrophic factor (BDNF), neurotrophins NT-3 and NT-4/5, ciliary neurotrophic factor (CNTF), glial cell line derived neurotrophic factor (GDNF), neurturin, agrin, any one of the family of semaphorins/collapsins, netrin-1 and netrin-2, hepatocyte growth factor (HGF), ephrins, noggin, sonic hedgehog and tyrosine hydroxylase.

In one or more embodiments, the therapeutic protein comprises immune system regulatory proteins, wherein the regulatory proteins comprises cytokines and lymphokines such as thrombopoietin (TPO), interleukins (IL) IL-1 through IL-25 (including, e.g., IL-2, IL-4, IL-12 and IL-18), monocyte chemoattractant protein, leukemia inhibitory factor, granulocyte-macrophage colony stimulating factor, Fas ligand, tumor necrosis factors α and β, interferons α, β, and γ, stem cell factor, flk-2/flt3 ligand.

In one or more embodiments, the therapeutic protein comprises immunoglobulins IgG, IgM, IgA, IgD and IgE, chimeric immunoglobulins, humanized antibodies, single chain antibodies, T cell receptors, chimeric T cell receptors, single chain T cell receptors, class I and class II MHC molecules, as well as engineered immunoglobulins and MHC molecules.

In one or more embodiments, the therapeutic protein comprises complement regulatory proteins, wherein the complement regulatory proteins comprise membrane cofactor protein (MCP), decay accelerating factor (DAF), CR1, CF2 and CD59.

In one or more embodiments, the therapeutic protein comprises receptors for the hormones, growth factors, cytokines, lymphokines, regulatory proteins and immune system proteins.

In one or more embodiments, the therapeutic protein comprises receptors for cholesterol regulation and/or lipid modulation, including the low-density lipoprotein (LDL) receptor, high density lipoprotein (HDL) receptor, the very low density lipoprotein (VLDL) receptor, and scavenger receptors.

In one or more embodiments, the therapeutic protein comprises members of the steroid hormone receptor superfamily including glucocorticoid receptors and estrogen receptors, Vitamin D receptors and other nuclear receptors.

In one or more embodiments, the therapeutic protein comprises transcription factors such as jun, fos, max, mad, serum response factor (SRF), AP-1, AP2, myb, MyoD and myogenin, ETS-box containing proteins, TFE3, E2F, ATF1, ATF2, ATF3, ATF4, ZFS, NFAT, CREB, HNF-4, C/EBP, SP1, CCAAT-box binding proteins, interferon regulation factor (IRF-1), Wilms tumor protein, ETS-binding protein, STAT, GATA-box binding proteins, and the forkhead family of winged helix proteins.

In one or more embodiments, the therapeutic protein comprises carbamoyl synthetase I, ornithine transcarbamylase, arginosuccinate synthetase, arginosuccinate lyase, arginase, fumarylacetacetate hydrolase, phenylalanine hydroxylase, alpha-1 antitrypsin, glucose-6-phosphatase, porphobilinogen deaminase, cystathione beta-synthase, branched chain ketoacid decarboxylase, albumin, isovaleryl-coA dehydrogenase, propionyl CoA carboxylase, methyl malonyl CoA mutase, glutaryl CoA dehydrogenase, insulin, beta-glucosidase, pyruvate carboxylate, hepatic phosphorylase, phosphorylase kinase, glycine decarboxylase, H-protein, T-protein, a cystic fibrosis transmembrane regulator (CFTR) sequence, and a dystrophin gene product.

In one or more embodiments, the therapeutic protein comprises CDKL5, alpha-galactosidase, β-galactosidase, β-hexosaminidase, galactosylceramidase, arylsulfatase, β-glucocerebrosidase, glucocerebrosidase, lysosomal acid lipase, lysosomal enzyme acid sphingomyelinase, formylglycine-generating enzyme, iduronidase, acetyl-CoA:alpha-glucosaminide N-acetyltransferase, glycosaminoglycan alpha-L-iduronohydrolase, heparan N-sulfatase, N-acetyl-α-D-glucosaminidase (NAGLU), iduronate-2-sulfatase, galactosamine-6-sulfate sulfatase, N-acetylgalactosamine-6-sulfatase, glycosaminoglycan N-acetylgalactosamine 4-sulfatase, β-glucuronidase, hyaluronidase, alpha-N-acetyl neuraminidase (sialidase), ganglioside sialidase, phosphotransferase, alpha-glucosidase, alpha-D-mannosidase, beta-D-mannosidase, aspartylglucosaminidase, alpha-L-fucosidase, battenin, palmitoyl protein thioesterases, and other Batten-related proteins, or an enzymatically active fragment thereof. In one or more embodiments, the Batten-related protein comprises ceroid-lipofuscinosis neuronal protein 1, ceroid-lipofuscinosis neuronal protein 2, ceroid-lipofuscinosis neuronal protein 3, ceroid-lipofuscinosis neuronal protein 4, ceroid-lipofuscinosis neuronal protein 5, ceroid-lipofuscinosis neuronal protein 6, ceroid-lipofuscinosis neuronal protein 7, ceroid-lipofuscinosis neuronal protein 8, ceroid-lipofuscinosis neuronal protein 19, ceroid-lipofuscinosis neuronal protein 10, ceroid-lipofuscinosis neuronal protein 11, ceroid-lipofuscinosis neuronal protein 12, ceroid-lipofuscinosis neuronal protein 13 and ceroid-lipofuscinosis neuronal protein 14.

In one or more embodiments, the therapeutic protein comprises proteins those used for treatment of hemophilia, including hemophilia B and hemophilia A.

In one or more embodiments, the therapeutic protein comprises non-naturally occurring polypeptides, such as chimeric or hybrid polypeptides having a non-naturally occurring amino acid sequence containing insertions, deletions or amino acid substitutions. In one or more embodiments, the non-naturally occurring polypeptide comprises single chain engineered immunoglobulins, antisense molecules and catalytic nucleic acids.

In one or more embodiments, the therapeutic protein comprises oncogene products comprising target antigens and target polypeptides, wherein target polypeptides comprises proteins which are produced exclusively or at higher levels in hyperproliferative cells as compared to normal cells and target antigens comprises proteins encoded by oncogenes including myb, myc, fyn, and the translocation gene bcr/abl, ras, src, P53, neu, trk and EGRF. In one or more embodiments, the therapeutic protein comprises variable regions of antibodies made by B cell lymphomas and variable regions of T cell receptors of T cell lymphomas. In one or more embodiments, the therapeutic protein comprises other tumor-associated polypeptides, wherein the polypeptides are found at higher levels in tumor cells including the polypeptide recognized by monoclonal antibody 17-1A and folate binding polypeptides.

In one or more embodiments, the therapeutic protein comprises protein that is useful for treating an individual suffering from autoimmune disease and disorder by conferring a broad based protective immune response against target that is associated with autoimmunity including cell receptor and cell which produce “self”-directed antibody. In one or more embodiments, the autoimmunity associated cell is T cell. In one or more embodiments, the autoimmune disease comprises Rheumatoid arthritis (RA), multiple sclerosis (MS), Sjögren's syndrome, sarcoidosis, insulin dependent diabetes mellitus (IDDM), autoimmune thyroiditis, reactive arthritis, ankylosing spondylitis, scleroderma, polymyositis, dermatomyositis, psoriasis, vasculitis, Wegener's granulomatosis, Crohn's disease and ulcerative colitis.

In one or more embodiments, the fusion protein comprises a third linker peptide, wherein the third linker peptide is located between the vesicle targeting protein and the protein of interest. In some embodiments, the third linker peptide is located at the terminal end of the fusion protein.

In some embodiments, the third linker peptide comprises one or more of a spacer peptide, a transmembrane domain and extravesicular domain. In some embodiments, the third linker peptide includes one or more of an affinity tag, a connector peptide, protease cleavage sites, a secretion signal peptide, cellular targeting domains, reporters, enzymes, or combination thereof. In some embodiments, the affinity tag may be one or more of mCherry, TwinStrep, polyhistidine, HA, FLAG, GST, and GFP.

In one or more embodiments, the fusion protein comprises more than one effector domains (ED). In some embodiments, the more than one effector domains (ED) comprises a first effector domain (ED) and a second effector domain (ED). In some embodiments, the first effector domain (ED) is directly connected to the second effector domain (ED). In some embodiments, the first effector domain (ED) is connected to the second effector domain (ED) via a linker peptide.

In some embodiments, proteolytic cleavage sites may be engineered into the fusion protein to promote the release of the protein of interest from the vesicular targeting protein and/or other peptide functional domains, including affinity tags, in conjunction with fusion protein synthesis or purification. Exemplary protease cleavage sites include, but are not limited to, cleavage sites sensitive to thrombin, furin, factor Xa, metalloproteases, enterokinases, and cathepsin. In some embodiments, the cleavage site comprises a lysocleave site. The lysocleave site is sensitive to an unknown lysosomal protease.

The cellular targeting domain may comprise amino acid sequences conferring cell-type specific or cell differentiation-specific targeting. The cellular targeting domain may be incorporated into the fusion protein or it can be fused to a co-expressed membrane-bound exosomal marker protein. Preferably the cellular targeting domain is fused to an extracellular domain in the membrane-bound protein. The cellular targeting domain may comprise an antibody or antibody derivative, a peptide ligand, a receptor ligand, a receptor fragment, a hormone, etc. Exemplary membrane-bound exosomal marker proteins include, but are not limited to tetraspanins, such as CD9, CD63, CD81, CD82, and CD151, and a variety of GPI (glycerol-phosphatidyl inositol)-anchored proteins, among others.

Exemplary antibody or antibody derived cellular targeting domains may include any member of the group consisting of: IgG, antibody variable region; isolated CDR region; single chain Fv molecule (scFv) comprising a VH and VL domain linked by a peptide linker allowing for association between the two domains to form an antigen binding site; bispecific scFv dimer; minibody comprising a scFv joined to a CH3 domain, single chain diabody fragment, dAb fragment, which consists of a VH or a VL domain; Fab fragment consisting of VL, VH, CL and CH1 domains; Fab′ fragment, which differs from a Fab fragment by the addition of a few residues at the carboxyl terminus of the heavy chain CH1 domain, including one or more cysteines from the antibody hinge region; Fab′-SH fragment, which is a Fab′ fragment in which the cysteine residue(s) of the constant domains bear a free thiol group; F(ab′)2, bivalent fragment comprising two linked Fab fragments; Fd fragment consisting of VH and CH1 domains; derivatives thereof, and any other antibody fragment(s) retaining antigen-binding function. Fv, scFv, or diabody molecules may be stabilized by the incorporation of disulfide bridges linking the VH and VL domains. When using antibody-derived targeting agents, any or all of the cellular targeting domains therein and/or Fc regions may be “humanized” using methodologies well known to those of skill in the art.

Functional domains in the fusion proteins of the present invention may be separated from one another by a spacer to facilitate the independent folding of each peptide portion relative to one another and ensure that the individual peptide portions in a fusion protein do not interfere with one another. The spacer may include any amino acid or mixtures thereof. Preferably, a chosen spacer will increase the flexibility of the protein and facilitate adoption of an extended conformation. Preferred peptide spacers comprise proline, lysine, glycine, alanine, and serine, or combinations thereof. In one embodiment, the linker is a glycine rich linker.

Spacer sequences are typically 1 to 10 amino acids in length, typically 1 to 8 amino acids in length such as 2, 3 or 4 amino acids in length. Suitable amino acids for incorporation in spacers are alanine, arginine, serine or glycine. Suitable spacers include Ala-Arg and Ser-Gly-Gly. In the practice of the present invention, the targeting moiety is introduced into the exosome by the expression of the fusion protein comprising the targeting moiety and exosomal transmembrane protein within a cell transduced with an rAAV-transgene, thus, producing the exosomes in vivo. Expression of this fusion protein in the cell, allows for the fusion protein to be incorporated into the exosome as it is produced from the cell. In a particular embodiment, the spacer is a 4 amino acid peptide.

In some embodiments, the fusion protein may comprise one or more curvature sensing domains. In one or more embodiment, the curvature sensing domain comprises SNX-1 curvature sensing domain. In some embodiments, the SNX-1 curvature sensing domain comprises the sequence according to SEQ ID NO: 154.

In one or more embodiments, the N-terminus of the fusion protein does not have methionine.

The disclosure also provides a polynucleotide sequence encoding the fusion protein.

One skilled in the art can readily derive a polynucleotide sequence encoding an amino acid sequence. Also, the polynucleotide sequence can be codon optimized for expression in target cells using commercially available products. The present invention includes the polynucleotide sequence encoding the fusion protein. The polynucleotide sequence encoding the fusion protein may be operably linked to expression control sequences, e.g., a promoter that directs expression of the gene, a secretion signal peptide gene.

In one or more embodiments, the fusion protein is conjugated with lipids. In some embodiments, the fusion protein is conjugated by myristoylation, palmitoylation, prenylation or glycosylphosphatidylinositol anchor protein.

In some embodiments, the fusion protein is further reconstituted into a lipid vesicle.

### Expressing the Fusion Protein

In one or more embodiments, the method of producing the fusion protein comprises transfecting host cells, allowing the host cells to transiently express the fusion protein, isolating exosomes, and purifying the fusion protein from. In one or more embodiments, the purified fusion protein is loaded into exosomes. In one or more embodiments, exosomes are produces exogenously. In one or more embodiments, the fusion protein is expressed in host cells. in one or more embodiments, the host cells comprise any one of Expi293F cells, PC12 cells, HAP1 cells, Chinese hamster ovary (CHO) cells, HeLa cells, human embryonic kidney (HEK) cells, mouse primary myoblasts, NIH 3T3 cells, or Escherichia coli cells. In one or more embodiments, cell-free expression synthesis is used to express the fusion protein.

In one or more embodiments, the fusion protein expressing cells are induced to increase production of exosomes. In some embodiments, the fusion protein expressing cells are induced with N-methyldopamine and/or norepinephrine. In some embodiments, the fusion protein expressing cells are induced with a cytotoxic agent. In some embodiments, the cytotoxic agent comprises melphalan.

In one or more embodiments, the host cells are transfected using suitable vectors, wherein the vector comprises viruses (such as adenoviruses, adeno-associated virus (AAV), vaccinia, herpesviruses, baculoviruses and retroviruses, parvovirus, lentivirus) bacteriophages, cosmids, plasmids, fungal vectors, naked DNA, DNA lipid complexes, and other recombination vehicles typically used in the art which have been described for expression in a variety of eukaryotic and prokaryotic hosts, and may be used for gene therapy as well as for simple protein expression.

### Regulatory Elements

In addition to the transgene, the minigene can also include conventional control elements which are operably linked to the transgene in a manner which permits its transcription, translation and/or expression in a cell transfected with the plasmid vector or infected with the virus produced by the invention. As used herein, “operably linked” sequences include both expression control sequences that are contiguous with the gene of interest and expression control sequences that act in trans or at a distance to control the gene of interest.

Expression control sequences include appropriate transcription initiation, termination, promoter and enhancer sequences; efficient RNA processing signals such as splicing and polyadenylation (polyA) signals; sequences that stabilize cytoplasmic mRNA; sequences that enhance translation efficiency (i.e., Kozak consensus sequence); sequences that enhance protein stability; and when desired, sequences that enhance secretion of the encoded product. A great number of expression control sequences, including promoters which are native, constitutive, inducible and/or tissue-specific, are known in the art and may be utilized.

Examples of constitutive promoters include, without limitation, the retroviral Rous sarcoma virus (RSV) LTR promoter (optionally with the RSV enhancer), the cytomegalovirus (CMV) promoter (optionally with the CMV enhancer) [see, e.g., Boshart et al, Cell, 41:521-530 (1985)], the SV40 promoter, the dihydrofolate reductase promoter, the 13-actin promoter, the phosphoglycerol kinase (PGK) promoter, and the EF1 promoter [Invitrogen]. Inducible promoters allow regulation of gene expression and can be regulated by exogenously supplied compounds, environmental factors such as temperature, or the presence of a specific physiological state, e.g., acute phase, a particular differentiation state of the cell, or in replicating cells only. Inducible promoters and inducible systems are available from a variety of commercial sources, including, without limitation, Invitrogen, Clontech and Ariad. Many other systems have been described and can be readily selected by one of skill in the art. Examples of inducible promoters regulated by exogenously supplied compounds, include, the zinc-inducible sheep metallothionine (MT) promoter, the dexamethasone (Dex)-inducible mouse mammary tumor virus (MMTV) promoter, the T7 polymerase promoter system [International Patent Publication No. WO 98/10088]; the ecdysone insect promoter [No et al, Proc. Natl. Acad. Sci. USA, 93:3346-3351 (1996)], the tetracycline-repressible system [Gossen et al, Proc. Natl. Acad. Sci. USA, 89:5547-5551 (1992)], the tetracycline-inducible system [Gossen et al, Science, 268:1766-1769 (1995), see also Harvey et al, Curr. Opin. Chem. Biol., 2:512-518 (1998)], the RU486-inducible system [Wang et al, Nat. Biotech., 15:239-243 (1997) and Wang et al, Gene Ther., 4:432-441 (1997)] and the rapamycin-inducible system [Magari et al, J. Clin. Invest., 100:2865-2872 (1997)]. Other types of inducible promoters which may be useful in this context are those which are regulated by a specific physiological state, e.g., temperature, acute phase, a particular differentiation state of the cell, or in replicating cells only.

In another embodiment, the native promoter for the transgene will be used. The native promoter may be preferred when it is desired that expression of the transgene should mimic the native expression. The native promoter may be used when expression of the transgene must be regulated temporally or developmentally, or in a tissue-specific manner, or in response to specific transcriptional stimuli. In a further embodiment, other native expression control elements, such as enhancer elements, polyadenylation sites or Kozak consensus sequences may also be used to mimic the native expression.

Another embodiment of the transgene includes a gene operably linked to a tissue-specific promoter. For instance, if expression in skeletal muscle is desired, a promoter active in muscle should be used. These include the promoters from genes encoding skeletal β-actin, myosin light chain 2A, dystrophin, muscle creatine kinase, as well as synthetic muscle promoters with activities higher than naturally-occurring promoters (see Li et al., Nat. Biotech., 17:241-245 (1999)). Examples of promoters that are tissue-specific are known for liver (albumin, Miyatake et al., J. Virol., 71:5124-32 (1997); hepatitis B virus core promoter, Sandig et al., Gene Ther., 3:1002-9 (1996); alpha-fetoprotein (AFP), Arbuthnot et al., Hum. Gene Ther., 7:1503-14 (1996)), bone osteocalcin (Stein et al., Mol. Biol. Rep., 24:185-96 (1997)); bone sialoprotein (Chen et al., J. Bone Miner. Res., 11:654-64 (1996)), lymphocytes (CD2, Hansal et al., J. Immunol., 161:1063-8 (1998); immunoglobulin heavy chain; T cell receptor chain), neuronal such as neuron-specific enolase (NSE) promoter (Andersen et al., Cell. Mol. Neurobiol., 13:503-15 (1993)), neurofilament light-chain gene (Piccioli et al., Proc. Natl. Acad. Sci. USA, 88:5611-5 (1991)), and the neuron-specific vgf gene (Piccioli et al., Neuron, 15:373-84 (1995)), among others.

Optionally, plasmids carrying therapeutically useful transgenes may also include selectable markers or reporter genes may include sequences encoding geneticin, hygromicin or purimycin resistance, among others. Such selectable reporters or marker genes (preferably located outside the viral genome to be rescued by the method of the invention) can be used to signal the presence of the plasmids in bacterial cells, such as ampicillin resistance. Other components of the plasmid may include an origin of replication. Selection of these and other promoters and vector elements are conventional, and many such sequences are available [see, e.g., Sambrook et al, and references cited therein].

### Delivery of the Minigene to a Packaging Host Cell

The minigene can be carried on any suitable vector, e.g., a plasmid, which is delivered to a host cell. The plasmids useful in this invention may be engineered such that they are suitable for replication and, optionally, integration in prokaryotic cells, mammalian cells, or both. These plasmids (or other vectors carrying the 5′ AAV ITR-heterologous molecule-3′ AAV ITR) contain sequences permitting replication of the minigene in eukaryotes and/or prokaryotes and selection markers for these systems. Selectable markers or reporter genes may include sequences encoding geneticin, hygromicin or purimycin resistance, among others. The plasmids may also contain certain selectable reporters or marker genes that can be used to signal the presence of the vector in bacterial cells, such as ampicillin resistance. Other components of the plasmid may include an origin of replication and an amplicon, such as the amplicon system employing the Epstein Barr virus nuclear antigen. This amplicon system, or other similar amplicon components permit high copy episomal replication in the cells. Preferably, the molecule carrying the minigene is transfected into the cell, where it may exist transiently. Alternatively, the minigene (carrying the 5′ AAV ITR-heterologous molecule-3′ ITR) may be stably integrated into the genome of the host cell, either chromosomally or as an episome. In certain embodiments, the minigene may be present in multiple copies, optionally in head-to-head, head-to-tail, or tail-to-tail concatemers. Suitable transfection techniques are known and may readily be utilized to deliver the minigene to the host cell.

Generally, when delivering the vector comprising the minigene by transfection, the vector is delivered in an amount from about 5 μg to about 100m DNA, about 10 μg to about 50 μg DNA to about 1×10⁴ cells to about 1×10¹³ cells, or about 1×10⁵ cells. However, the relative amounts of vector DNA to host cells may be adjusted, taking into consideration such factors as the selected vector, the delivery method and the host cells selected.

### A. Rep and Cap Sequences

In addition to the minigene, the packaging host cell contains the sequences which drive expression of a novel AAV capsid protein (or a capsid protein comprising a fragment thereof) in the host cell and rep sequences of the same source as the source of the AAV ITRs found in the minigene, or a cross-complementing source. The AAV cap and rep sequences may be independently obtained from an AAV source as described above and may be introduced into the host cell in any manner known to one in the art as described above. Additionally, when pseudo typing an AAV vector in (e.g., an AAV9/HU.14 capsid), the sequences encoding each of the essential rep proteins may be supplied by different AAV sources (e.g., AAV1, AAV2, AAV3, AAV4, AAV5, AAV6, AAV7, AAV8). For example, the rep78/68 sequences may be from AAV2, whereas the rep52/40 sequences may be from AAV8.

In one embodiment, the host cell stably contains the capsid protein under the control of a suitable promoter, such as those described above. Most desirably, in this embodiment, the capsid protein is expressed under the control of an inducible promoter. In another embodiment, the capsid protein is supplied to the host cell in trans. When delivered to the host cell in trans, the capsid protein may be delivered via a plasmid which contains the sequences necessary to direct expression of the selected capsid protein in the host cell. Most desirably, when delivered to the host cell in trans, the plasmid carrying the capsid protein also carries other sequences required for packaging the rAAV, e.g., the rep sequences.

In another embodiment, the host cell stably contains the rep sequences under the control of a suitable promoter, such as those described above. Most desirably, in this embodiment, the essential rep proteins are expressed under the control of an inducible promoter. In another embodiment, the rep proteins are supplied to the host cell in trans. When delivered to the host cell in trans, the rep proteins may be delivered via a plasmid which contains the sequences necessary to direct expression of the selected rep proteins in the host cell. Most desirably, when delivered to the host cell in trans, the plasmid carrying the capsid protein also carries other sequences required for packaging the rAAV, e.g., the rep and cap sequences.

Thus, in one embodiment, the rep and cap sequences may be transfected into the host cell on a single nucleic acid molecule and exist stably in the cell as an episome. In another embodiment, the rep and cap sequences are stably integrated into the chromosome of the cell. Another embodiment has the rep and cap sequences transiently expressed in the host cell. For example, a useful nucleic acid molecule for such transfection comprises, from 5′ to 3′, a promoter, an optional spacer interposed between the promoter and the start site of the rep gene sequence, an AAV rep gene sequence, and an AAV cap gene sequence.

Optionally, the rep and/or cap sequences may be supplied on a vector that contains other DNA sequences that are to be introduced into the host cells. For instance, the vector may contain the rAAV construct comprising the minigene. The vector may comprise one or more of the genes encoding the helper functions, e.g., the adenoviral proteins E1, E2a, and E4 ORF6, and the gene for VAI RNA.

Preferably, the promoter used in this construct may be any of the constitutive, inducible or native promoters known to one of skill in the art or as discussed above. In one embodiment, an AAV P5 promoter sequence is employed. The selection of the AAV to provide any of these sequences does not limit the invention.

In another preferred embodiment, the promoter for rep is an inducible promoter, such as are discussed above in connection with the transgene regulatory elements. One preferred promoter for rep expression is the T7 promoter. The vector comprising the rep gene regulated by the T7 promoter and the cap gene, is transfected or transformed into a cell which either constitutively or inducibly expresses the T7 polymerase. See International Patent Publication No. WO 98/10088, published Mar. 12, 1998.

The spacer is an optional element in the design of the vector. The spacer is a DNA sequence interposed between the promoter and the rep gene ATG start site. The spacer may have any desired design; that is, it may be a random sequence of nucleotides, or alternatively, it may encode a gene product, such as a marker gene. The spacer may contain genes which typically incorporate start/stop and polyA sites. The spacer may be a non-coding DNA sequence from a prokaryote or eukaryote, a repetitive non-coding sequence, a coding sequence without transcriptional controls or a coding sequence with transcriptional controls. Two exemplary sources of spacer sequences are the phage ladder sequences or yeast ladder sequences, which are available commercially, e.g., from Gibco or Invitrogen, among others. The spacer may be of any size sufficient to reduce expression of the rep78 and rep68 gene products, leaving the rep52, rep40 and cap gene products expressed at normal levels. The length of the spacer may therefore range from about 10 bp to about 10.0 kbp, preferably in the range of about 100 bp to about 8.0 kbp. To reduce the possibility of recombination, the spacer is preferably less than 2 kbp in length; however, the invention is not so limited.

Although the molecule(s) providing rep and cap may exist in the host cell transiently (i.e., through transfection), it is preferred that one or both of the rep and cap proteins and the promoter(s) controlling their expression be stably expressed in the host cell, e.g., as an episome or by integration into the chromosome of the host cell. The methods employed for constructing embodiments of this invention are conventional genetic engineering or recombinant engineering techniques such as those described in the references above. While this specification provides illustrative examples of specific constructs, using the information provided herein, one of skill in the art may select and design other suitable constructs, using a choice of spacers, P5 promoters, and other elements, including at least one translational start and stop signal, and the optional addition of polyadenylation sites.

In another embodiment of this invention, the rep or cap protein may be provided stably by a host cell.

### The Helper Functions

The packaging host cell also requires helper functions in order to package the rAAV of the invention. Optionally, these functions may be supplied by a herpesvirus. Most desirably, the necessary helper functions are each provided from a human or non-human primate adenovirus source, such as those described above and/or are available from a variety of sources, including the American Type Culture Collection (ATCC), Manassas, Va. (US). In one currently preferred embodiment, the host cell is provided with and/or contains an Ela gene product, an E1b gene product, an E2a gene product, and/or an E4 ORF6 gene product. The host cell may contain other adenoviral genes such as VAI RNA, but these genes are not required. In a preferred embodiment, no other adenovirus genes or gene functions are present in the host cell.

By “adenoviral DNA which expresses the Ela gene product”, it is meant any adenovirus sequence encoding E1a or any functional Ela portion. Adenoviral DNA which expresses the E2a gene product and adenoviral DNA which expresses the E4 ORF6 gene products are defined similarly. Also included are any alleles or other modifications of the adenoviral gene or functional portion thereof. Such modifications may be deliberately introduced by resort to conventional genetic engineering or mutagenic techniques to enhance the adenoviral function in some manner, as well as naturally occurring allelic variants thereof. Such modifications and methods for manipulating DNA to achieve these adenovirus gene functions are known to those of skill in the art.

The adenovirus Ela, E1b, E2a, and/or E4ORF6 gene products, as well as any other desired helper functions, can be provided using any means that allows their expression in a cell. Each of the sequences encoding these products may be on a separate vector, or one or more genes may be on the same vector. The vector may be any vector known in the art or disclosed above, including plasmids, cosmids and viruses. Introduction into the host cell of the vector may be achieved by any means known in the art or as disclosed above, including transfection, infection, electroporation, liposome delivery, membrane fusion techniques, high velocity DNA-coated pellets, viral infection and protoplast fusion, among others. One or more of the adenoviral genes may be stably integrated into the genome of the host cell, stably expressed as episomes, or expressed transiently. The gene products may all be expressed transiently, on an episome or stably integrated, or some of the gene products may be expressed stably while others are expressed transiently. Furthermore, the promoters for each of the adenoviral genes may be selected independently from a constitutive promoter, an inducible promoter or a native adenoviral promoter. The promoters may be regulated by a specific physiological state of the organism or cell (i.e., by the differentiation state or in replicating or quiescent cells) or by exogenously added factors.

### Host Cells And Packaging Cell Lines

The host cell itself may be selected from any biological organism, including prokaryotic (e.g., bacterial) cells, and eukaryotic cells, including, insect cells, yeast cells and mammalian cells. Particularly desirable host cells are selected from among any mammalian species, including, without limitation, cells such as A549, WEHI, 3T3, 10T1/2, BHK, MDCK, COS 1, COS 7, BSC 1, BSC 40, BMT 10, VERO, WI38, HeLa, 293 cells (which express functional adenoviral E1), Saos, C2C12, L cells, HT1080, HepG2 and primary fibroblast, hepatocyte and myoblast cells derived from mammals including human, monkey, mouse, rat, rabbit, and hamster. The selection of the mammalian species providing the cells is not a limitation of this invention; nor is the type of mammalian cell, i.e., fibroblast, hepatocyte, tumor cell, etc. The requirements for the cell used is that it not carry any adenovirus gene other than E1, E2a and/or E4 ORF6; it not contain any other virus gene which could result in homologous recombination of a contaminating virus during the production of rAAV; and it is capable of infection or transfection of DNA and expression of the transfected DNA. In a preferred embodiment, the host cell is one that has rep and cap stably transfected in the cell.

One host cell useful in the present invention is a host cell stably transformed with the sequences encoding rep and cap, and which is transfected with the adenovirus E1, E2a, and E4ORF6 DNA and a construct carrying the minigene as described above. Stable rep and/or cap expressing cell lines, such as B-50 (International Patent Application Publication No. WO 99/15685), or those described in U.S. Pat. No. 5,658,785, may also be similarly employed. Another desirable host cell contains the minimum adenoviral DNA which is sufficient to express E4 ORF6. Yet other cell lines can be constructed using the novel AAV9 cap sequences of the invention.

One platform for producing rAAV involves transfecting HEK293 cells with either two or three plasmids; one encoding the gene of interest, one carrying the AAV rep/cap genes, and another containing helper genes provided by either adeno or herpes viruses. Many robust production levels have been achieved with adherent cells in either roller bottles or cell stacks. Additionally, high production levels can be achieved in suspension-adapted HEK293 cells. Production is not limited to that described, but can incorporated improved production technologies that are known in the art.

Introduction of the molecules (as plasmids or viruses) into the packaging host cell may also be accomplished using techniques known to the skilled artisan and as discussed throughout the specification. In preferred embodiment, standard transfection techniques are used, e.g., CaPO4 transfection or electroporation, and/or infection by hybrid adenovirus/AAV vectors into cell lines such as the human embryonic kidney cell line HEK 293 (a human kidney cell line containing functional adenovirus E1 genes which provides trans-acting E1 proteins).

One of skill in the art will readily understand that the AAV sequences can be readily adapted for in vitro, ex vivo or in vivo gene delivery. Similarly, one of skill in the art can readily select other fragments of the AAV genome for use in a variety of rAAV and non-rAAV vector systems. Such vectors systems may include, e.g., lentiviruses, retroviruses, poxviruses, vaccinia viruses, and adenoviral systems, among others. Selection of these vector systems is not a limitation of the present invention.

### Purification of Vesicle Directed Protein

After expression and secretion, exosomes and extracellular vesicles containing the fusion protein can be isolated and purified from the surrounding cell culture media using standard techniques. In some embodiments, the exosomes and extracellular vesicles may be purified by precipitating and washing. In some embodiments, the purified exosomes and extracellular vesicles can be further lysed to purify the fusion protein from within.

### Modification of the Purified Fusion Protein

In one or more embodiments, the purified fusion protein is subject to lipid conjugation. In one or more embodiments, the lipid conjugation process comprises myristoylation. In one or more embodiments, the lipid conjugation process comprises palmitoylation. In one or more embodiments, the lipid conjugation process comprises prenylation. In one or more embodiments, the lipid conjugation process comprises glycosylphosphatidylinositol anchor protein.

In one or more embodiments, the fusion protein or lipid conjugated fusion protein is reconstituted into a lipid vesicle. In some embodiments, the lipid vesicle is an exosome or a synthetic vesicle.

### Determining the Cross-Correction Efficiency of the Fusion Protein

Designing mechanisms by a protein or gene product produced in one cell can enter and affect non-transduced cells, called cross-correction, is a viable strategy to improve safety and efficacy. Particularly, cross-correction maximizes the therapeutic effect by delivering the therapeutic macromolecule to cells that have not been transduced. This way, only a minority of cells need to be transduced to have a potential effect on the entire tissue. The viral vector dosage can be reduced, thereby reducing the risk for adverse events and reducing the cost per dose.

In one or more embodiments, the cross-correction efficiency for the fusion protein is measured, wherein cells transfected with vector encoding the fusion protein with fluorescence protein tag are co-cultured with untransfected cells and visualized under the fluorescence microscope. In some embodiments, the cells are COS-7. In some embodiments, the fluorescence protein tag is mCherry tag. In some embodiments, the therapeutic protein of the fusion protein comprises CDKL5. In some embodiments, the therapeutic protein of the fusion protein comprises alpha-galactosidase A.

In one or more embodiments, the cross-correction efficiency for the fusion protein is measured in vivo. In some embodiments, the fusion protein comprises MyrED27 (SEQ ID NO: 57), MyrED77 (SEQ ID NO: 107), MyrED81 (SEQ ID NO: 111) or MyrED88 (SEQ ID NO: 118). In some embodiments, the in vivo study comprises using mouse embryos. Accordingly, in some embodiments, the embryo brains inside the uterus are directly injected with plasmids and/or electroporated enabling the plasmids to enter brain cells. In some embodiments, a positive control is used to determine relative transduction efficiency. In some embodiments, the positive control construct comprises BiP-mCherry-IRES-GFP. In some embodiments, the plasmids are such that transfected cells express GFP and mCherry, whereas cross-corrected cells only contain mCherry. Cross-corrected cells do not express mCherry, they receive an already translated protein from the transduced cell. In some embodiments, the embryos are then harvested four days later at E18.5. In some embodiments, the embryonic brain sections are fluorescently imaged for presence of GFP and mCherry and compared against the positive control.

### Determining Relative Expression of the Fusion Protein Containing Membrane Associated-ED Variants

In some embodiments, the relative expression of the fusion protein containing membrane associated-ED variants in exosomes can be determined by western blot analysis. In some embodiments, the cells can be COS-7 or Expi293. In one or more embodiments, the fluorescence protein tag may be mCherry tag.

### Delivering the Fusion Protein to a Patient

Another aspect of the present invention relates to a pharmaceutical formulation comprising the fusion protein and a pharmaceutically acceptable carrier.

Another aspect of the present invention relates to a method of treating a disease or disorder comprising administering the pharmaceutical formulation to a patient in need thereof. In some embodiments, the disease or disorder comprises a CDKL5-mediated neurological disorder and the therapeutic protein comprises CDKL5. In some embodiments, the disease or disorder comprises Fabry disease and the therapeutic protein comprises alpha-galactosidase A. In some embodiments, the disease or disorder comprises Pompe disease and the therapeutic protein comprises alpha-glucosidase.

In one or more embodiments, the pharmaceutical composition is administered as a prophylactic to prevent a disease or disorder.

In one or more embodiments, the pharmaceutical formulation is administered intrathecally, intracranially, intra cisterna magnally, intravenously, intracisternally, intracerebroventrically, or intraparenchymally.

### Gene Therapy

Another aspect of the present invention is application in gene therapy. In some embodiments, a gene therapy composition comprises a gene therapy delivery system and a polynucleotide encoding an engineered protein. In some embodiments, the engineered protein comprises the fusion protein. In some embodiments, the gene therapy delivery system may be one or more of a vector, a liposome, a lipid-nucleic acid nanoparticle, an exosome, and a gene editing system. In some embodiments, the gene therapy delivery system comprises one or more of Clustered Regularly Interspaced Short Palindromic Repeat (CRISPR) associated protein 9 (CRISPR-Cas-9), Transcription activator-like effector nuclease (TALEN), or ZNF (Zinc finger protein). In some embodiments, the gene therapy delivery system comprises a promoter. In some embodiments, the gene therapy delivery system comprises a polynucleotide encoding a leader signal polypeptide.

In some embodiments, the gene therapy delivery system may be a viral vector. The viral vector includes one or more of an adenoviral vector, an adeno-associated viral vector, a lentiviral vector, a retroviral vector, a poxviral vector, or a herpes simplex viral vector. The viral vectors may also include additional elements for increasing expression and/or stabilizing the vector such as promoters (e.g., hybrid CBA promoter (CBh) and human synapsin 1 promoter (hSyn1)), a polyadenylation signals (e.g. Bovine growth hormone polyadenylation signal (bGHpolyA)), stabilizing elements (e.g. Woodchuck Hepatitis Virus (WHP) Posttranscriptional Regulatory Element (WPRE)) and/or an SV40 intron. In some embodiments, the viral vector comprises a viral polynucleotide operably linked to the polynucleotide encoding the engineered protein. In some embodiments, the viral vector comprises at least one inverted terminal repeat (ITR).

In one or more embodiments, the gene therapy composition comprises a pharmaceutically acceptable carrier. The pharmaceutically acceptable carrier includes a diluent, adjuvant, excipient, or vehicle with which the compound is administered

Various aspects of the present invention relate to a method of treating a disease or disorder comprising administering the gene therapy composition to a patient in need thereof. In one of more embodiments, the gene therapy composition is administered intrathecally, intracranially, intra cisterna magnally, intravenously, intracisternally, intracerebroventrically, or intraparenchymally.

### Recombinant Viruses And Uses Therefor

Using the techniques described herein, one of skill in the art can generate a rAAV having a capsid of an AAV provided herein and hosting a minigene that includes a desired transgene. In one embodiment, a full-length capsid from a single AAV can be utilized. In another embodiment, a full-length capsid may be generated which contains one or more fragments of the novel AAV capsid fused in frame with sequences from another selected AAV, or from heterologous (i.e., non-contiguous) portions of the same AAV. Alternatively, the unique AAV sequences may be used in constructs containing other viral or non-viral sequences.

### Delivery of Viruses

The rAAV containing a minigene, which includes the desired transgene, can be administered directly to the targeted cells for generating exosomes in vivo by one of a number of available methods of administration. For example, administration can be by one of a different number of administration routes including orally, parenterally, sublingually, transdermally, rectally, transmucosally, topically, via inhalation, via buccal administration, intrapleurally, intravenous, intraarterial, intraperitoneal, subcutaneous, intramuscular, intranasal, intralymphatically, periocularly, intrathecal, and intraarticular or combinations thereof. For veterinary use, the rAAV can be administered as a suitably acceptable formulation in accordance with normal veterinary practice. The veterinarian can readily determine the dosing regimen and route of administration that is most appropriate for a particular animal. Preferably, the rAAV vector containing the minigene with the transgene can be administered by traditional syringes, needleless injection devices, “microprojectile bombardment gene guns”, or other physical methods such as electroporation (“EP”), “hydrodynamic method”, or ultrasound.

In one embodiment, the invention provides a method for AAV-mediated delivery of a transgene to a host. This method involves transfecting or infecting a selected host cell with a recombinant viral vector containing a selected transgene under the control of sequences that direct expression thereof and AAV9 capsid proteins.

Optionally, a sample from the host may be first assayed for the presence of antibodies to a selected AAV source (e.g., a serotype). A variety of assay formats for detecting neutralizing antibodies are well known to those of skill in the art. The selection of such an assay is not a limitation of the present invention. See, e.g., Fisher et al, Nature Med, 3(3):306-312 (March 1997) and W C Manning et al, Human Gene Therapy, 9:477-485 (Mar. 1, 1998). The results of this assay may be used to determine which AAV vector containing capsid proteins of a particular source are preferred for delivery, e.g., by the absence of neutralizing antibodies specific for that capsid source.

In one aspect of this method, the delivery of vector with AAV capsid proteins may precede or follow delivery of a gene via a vector with a different AAV capsid protein. Thus, gene delivery via rAAV vectors may be used for repeat gene delivery to a selected host cell. Desirably, subsequently administered rAAV vectors carry the same transgene as the first rAAV vector, but the subsequently administered vectors contain capsid proteins of sources (and preferably, different serotypes) which differ from the first vector.

Optionally, multiple rAAV vectors can be used to deliver large transgenes or multiple transgenes by co-administration of rAAV vectors concatamerize in vivo to form a single vector genome. In such an embodiment, a first AAV may carry an expression cassette which expresses a single transgene (or a subunit thereof) and a second AAV may carry an expression cassette which expresses a second transgene (or a different subunit) for co-expression in the host cell. A first AAV may carry an expression cassette which is a first piece of a polycistronic construct (e.g., a promoter and transgene, or subunit) and a second AAV may carry an expression cassette which is a second piece of a polycistronic construct (e.g., transgene or subunit and a polyA sequence). These two pieces of a polycistronic construct concatamerize in vivo to form a single vector genome that co-expresses the transgenes delivered by the first and second AAV. In such embodiments, the rAAV vector carrying the first expression cassette and the rAAV vector carrying the second expression cassette can be delivered in a single pharmaceutical composition. In other embodiments, the two or more rAAV vectors are delivered as separate pharmaceutical compositions which can be administered substantially simultaneously, or shortly before or after one another.

The above-described recombinant vectors may be delivered to a subject according to published methods. The rAAV, preferably suspended in a physiologically compatible carrier, may be administered to a human or non-human mammalian patient. Suitable carriers may be readily selected by one of skill in the art in view of the indication for which the transfer virus is directed. For example, one suitable carrier includes saline, which may be formulated with a variety of buffering solutions (e.g., phosphate buffered saline). Other exemplary carriers include sterile saline, lactose, sucrose, calcium phosphate, gelatin, dextran, agar, pectin, peanut oil, sesame oil, and water. The selection of the carrier is not a limitation of the present invention.

Optionally, the compositions of the invention may contain, in addition to the rAAV and carrier(s), other conventional pharmaceutical ingredients, such as preservatives, or chemical stabilizers. Suitable exemplary preservatives include chlorobutanol, potassium sorbate, sorbic acid, sulfur dioxide, propyl gallate, the parabens, ethyl vanillin, glycerin, phenol, and parachlorophenol. Suitable chemical stabilizers include gelatin and albumin

The vectors are administered in sufficient amounts to transfect the cells and to provide sufficient levels of gene transfer and expression to provide a therapeutic benefit without undue adverse effects, or with medically acceptable physiological effects, which can be determined by those skilled in the medical arts. Conventional and pharmaceutically acceptable routes of administration include, but are not limited to, direct delivery to a desired organ (e.g., the liver (optionally via the hepatic artery) or lung), oral, inhalation, intranasal, intratracheal, intraarterial, intraocular, intravenous, intramuscular, subcutaneous, intradermal, and other parental routes of administration. Routes of administration may be combined, if desired.

Dosages of the viral vector will depend primarily on factors such as the condition being treated, the age, weight and health of the patient, and may thus vary among patients. For example, a therapeutically effective human dosage of the viral vector is generally in the range of from about 0.1 mL to about 100 mL of solution containing concentrations of from about 1×10⁹ to 1×10¹⁶ genomes virus vector. A preferred human dosage for delivery to large organs (e.g., liver, muscle, heart and lung) may be about 5×10¹⁰ to 5×10¹³ AAV genomes per 1 kg, at a volume of about 1 to 100 mL. A preferred dosage for delivery to eye is about 5×10⁹ to 5×10¹² genome copies, at a volume of about 0.1 mL to 1 mL. The dosage will be adjusted to balance the therapeutic benefit against any side effects and such dosages may vary depending upon the therapeutic application for which the recombinant vector is employed. The levels of expression of the transgene can be monitored to determine the frequency of dosage resulting in viral vectors, preferably AAV vectors containing the minigene. Optionally, dosage regimens similar to those described for therapeutic purposes may be utilized for immunization using the compositions of the invention.

Examples of therapeutic products and immunogenic products for delivery by the AAV-containing vectors are provided below. These vectors may be used for a variety of therapeutic or vaccinal regimens, as described herein. Additionally, these vectors may be delivered in combination with one or more other vectors or active ingredients in a desired therapeutic and/or vaccinal regimen.

### In Vivo Exosome Mediated Gene Delivery

Exosomes are produced by many different types of cells including immune cells such as B lymphocytes, T lymphocytes, dendritic cells (DCs) and most cells. Exosomes are also produced, for example, by glioma cells, platelets, reticulocytes, neurons, intestinal epithelial cells and tumor cells. Such cell types and the tissues that they define can be targeted for exosome production with the gene encoding the engineered protein. Such targeting is accomplished by transduction with the rAAV constructs containing the provided minigenes, including the gene encoding the engineered protein.

In a preferred aspect of the present invention, exosomes derived from DCs, preferably immature DCs are targeted. Exosomes produced from immature DCs do not express MHC-II, MHC-I or CD86. As such, such exosomes do not stimulate naive T cells to a significant extent and are unable to induce a response in a mixed lymphocyte reaction. Thus exosomes produced from immature dendritic cells are ideal candidates to target as a vehicle for delivery of genetic material, such as for gene therapy.

Nucleic acids for incorporation into the exosomes may be single or double stranded. Single-stranded nucleic acids include those with phosphodiester, 2′O-methyl, 2′ methoxy-ethyl, phosphoramidate, methylphosphonate, and/or phosphorothioate backbone chemistry. Typically double-stranded nucleic acids are introduced including for example plasmid DNA and small interfering RNAs (siRNAs).

### Targeting Exosomes (for Cross-Correction)

The present invention also relates to the targeting of exosomes to a desired cell type or tissue to enable cross-correction, as some cells such as muscle satellite, stem, or diseased (fibrosis or muscle degenerated) cells are difficult to transduce. This targeting is achieved by expressing on the surface of the exosome a targeting moiety which binds to a cell surface moiety expressed on the surface of the cell type needing cross-correction. Typically, the targeting moiety is a peptide which is expressed as an engineered protein with a transmembrane protein typically expressed on the surface of the exosome.

Suitable peptides for targeting cells for cross-correction are those which bind to cell surface moieties such as receptors or their ligands found on the cell surface of the cell to be targeted. Examples of suitable targeting moieties are short peptides, scFv and complete proteins, so long as the targeting moiety can be expressed on the surface of the exosome and does not interfere with insertion of the membrane protein into the exosome. Typically, the targeting peptide is heterologous to the transmembrane exosomal protein. Peptide targeting moieties may typically be less than 100 amino acids in length, for example less than 50 amino acids in length, less than 30 amino acids in length, to a minimum length of 10, 5 or 3 amino acids.

Targeting moieties can be selected to target particular tissue types such as muscle, brain, liver, pancreas and lung for example, or to target a diseased tissue such as a tumour. In a particularly preferred embodiment of the present invention, the exosomes are targeted to brain tissue.

Specific examples of targeting moieties include muscle specific peptide, discovered by phage display, to target skeletal muscle, a 29 amino acid fragment of Rabies virus glycoprotein that binds to the acetylcholine receptor or a fragment of neural growth factor that targets its receptor to target neurons and secretin peptide that binds to the secretin receptor can be used to target biliary and pancreatic epithelia. As an alternative, immunoglobulins and their derivatives, including scFv antibody fragments can also be expressed as an engineered protein to target specific antigens, such as VEGFR for cancer gene therapy. As an alternative, natural ligands for receptors can be expressed as engineered proteins to confer specificity, such as NGF which binds NGFR and confers neuron-specific targeting.

The peptide targeting moiety is expressed on the surface of the exosome by expressing it as an engineered protein with an exosomal transmembrane protein. In some embodiments, the exosomal transmembrane protein may be one or more of Lamp-1, Lamp-2, CD13, CD86, Flotillin, Syntaxin-3, CD2, CD36, CD40, CD40L, CD41a, CD44, CD45, ICAM-1, Integrin alpha4, LiCAM, LFA-1, Mac-1 alpha and beta, Vti-1A and B, CD3 epsilon and zeta, CD9, CD18, CD37, CD53, CD63, CD81, CD82, CXCR4, FcR, GluR2/3, HLA-DM (MHC II), immunoglobulins, MHC-I or MHC-II components, TCR beta and tetraspanins. In particularly preferred embodiments of the present invention, the transmembrane protein is selected from Lamp-1, Lamp-2, CD13, CD86, Flotillin, Syntaxin-3.

### Methods of Treatment

Provided herein are methods of treating, protecting against, and/or preventing a disease in a subject in need thereof by administering one or more rAAV vectors containing minigene comprising a desired transgene. Administration of the transgene to the subject can provide gene therapy to a subject via an exosome delivery pathway. This is particularly beneficial when the cells targeted for gene therapy have difficulty in transduction because they are either diseased, difficult to access (e.g., blood-brain barrier for brain cells), or highly susceptible to immune response, and thus, would particularly avail itself to delivery via an exosome as described herein.

Examples of diseases caused by genetic defect that are ideal for gene therapy are inherited retinal diseases (including voretigene neparvovec-RPE65), X-linked retinoschisis and achromatopsia, X-linked retinitis pigmentosa, and age-related macular degeneration, lipoprotein lipase (LPL) deficiency (LPL gene), Leber congenital amaurosis (eye disease) (RPE-specific protein RPE65 gene), choroideremia (eye disease) (REP-1 gene), achromatopsia (blindness) (CNGA3 gene), Leber's hereditary optic neuropathy (eye disease) (G11778A mitochondrial gene), X-linked juvenile retinoschisis (eye disease) (XLRS gene), hemophilia A (FVIII gene), hemophilia B (FIX gene), alpha-antitrypsin (AAT) deficiency (AAT gene), spinal muscular atrophy (SMN gene), Duchenne muscular dyystrophy (microDMD gene), limb girdle muscular dystrophy type 2D (alpha-sarcoglycan gene), Becker muscular dystrophy sporadic inclusion body myositis (Follistatin gene), dysferlin deficiency (dysferlin gene), rheumatoid arthritis (NF-kB and IFN-beta genes), metachromatic leukodystrophy (ARSA gene), Alzheimer's disease (NGF gene), idiopathic Parkinson's disease (Neurturin gene), gene enzyme replacement therapy (GERT) for AADC deficiency (AADC gene), GERT for LINCL (form of Batten disease) (TPP1 gene), GERT for MPSIIIA (Sanfilippo A syndrome) (SGSH gene), GERT to correct blood ammonia accumulation (OTC gene), and heptatis (heptatitis C virus), among other potential gene therapy targets.

### Kit

The disclosure provides a kit comprising the pharmaceutical composition and instructions for use. The disclosure also provides a kit comprising the gene therapy composition and instruction for use.

### EXAMPLES

### Example-1

### [A] Designing Truncated MARCKS Variant for Targeting the Fusion Protein to Exosomes

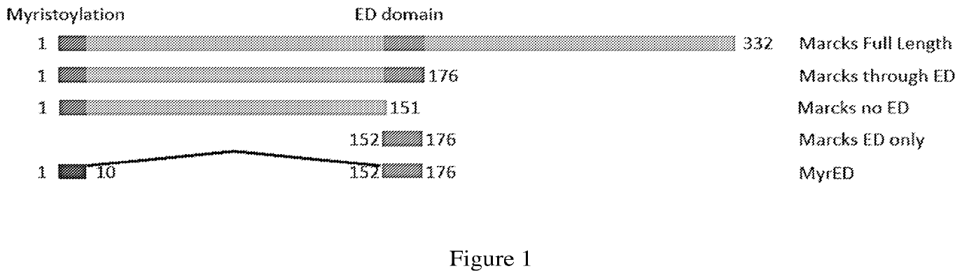

Four truncated variants of MARCKS protein, MARCKS through ED, MARCKS no ED, MARCKS ED only and MyrED, were designed to identify which part of the MARCKS protein is essential for targeting the fusion protein to the exosomes. FIG. 1 shows the schematic of four truncated MARCKS protein variants.

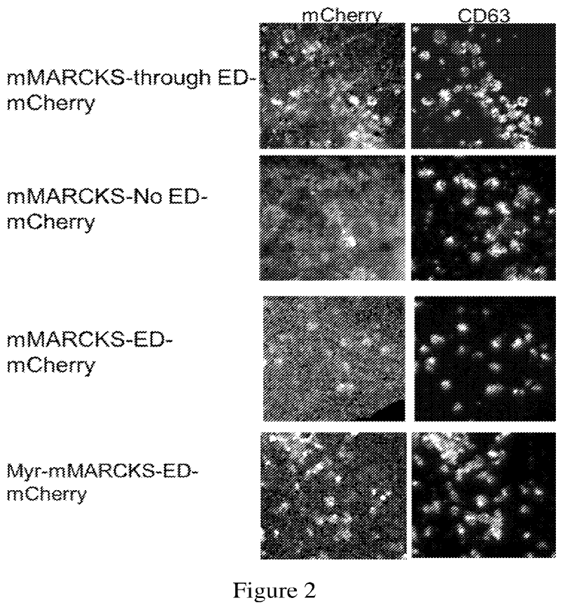

Each of the four truncated MARCKS protein variants were cloned with mCherry into a plasmid. Cos-7 cells were transfected with the plasmid. CD63 protein is targeted to exosomes. FIG. 2 is an immuno-visualized image for mCherry (left) and Anti-CD63 antibody directed against exosomal protein CD63 (right). The figure shows that mCherry fluorescence protein co-localizes with exosomal protein CD63 when fused to MARCKS through ED, MARCKS ED only, or MyrED.

### [B] Determining Cross-Correcting Ability of Truncated MARCKS for the Fusion Protein.

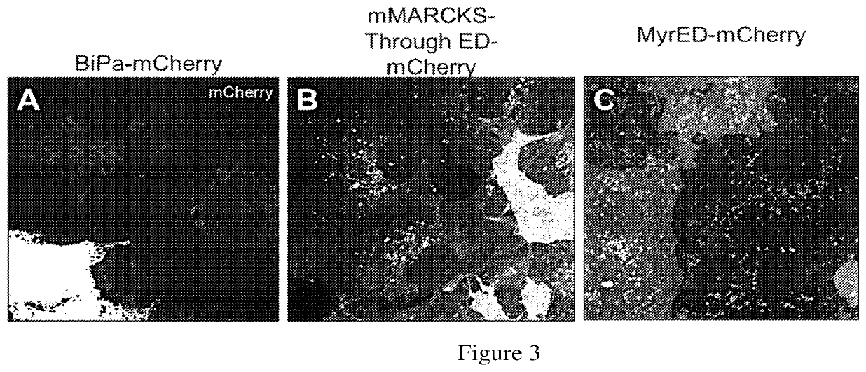

Two constructs, mMARCKS through ED and MyrED, with mCherry tag were created. Cos-7 cells were transfected with the construct. Cos-7 cells were also tranfected with BiPa, a secretory protein, and mCherry as a negative control. The transfected cells were co-cultured with non-transfected Cos-7 cells. FIG. 3 fluorescence image confirmed shows both constructs have ability to enter into neighboring untransfected cells.

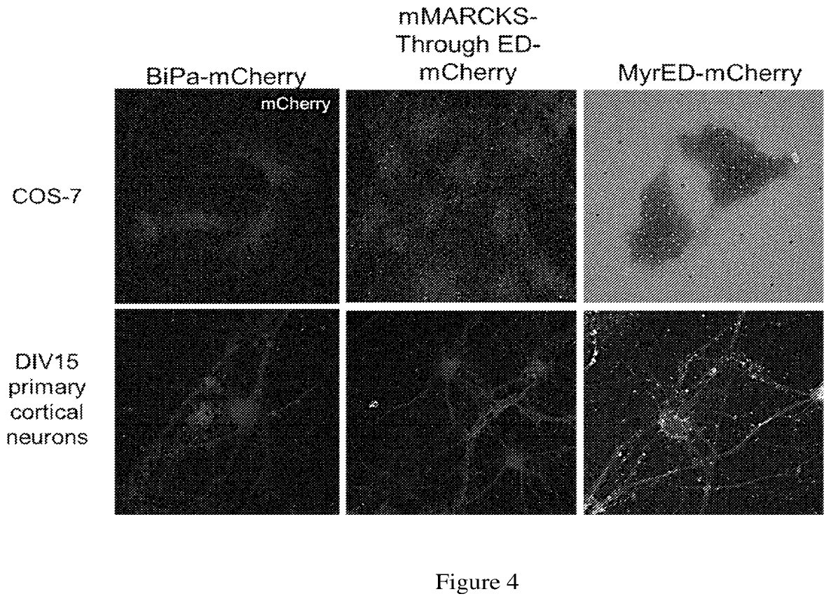

Both constructs, mMARCKS through ED and MyrED, with mCherry tag were used to transfect Cos-7 cells. For a secretory positive control, Cos-7 cells were transfected with BiPa tagged with mCherry. The transfected Cos-7 cells were co-cultured with non-transfected DIV15 primary cortical neuron cells that were treated with conditioned media produced from mCherry construct transfected 293T cells. FIG. 4 shows fluorescence image constructs having ability to enter into neighboring untransfected neuron cells.

### Example-2

### [A] Designing MyrED Variants for Targeting the Fusion Protein to Exosomes.

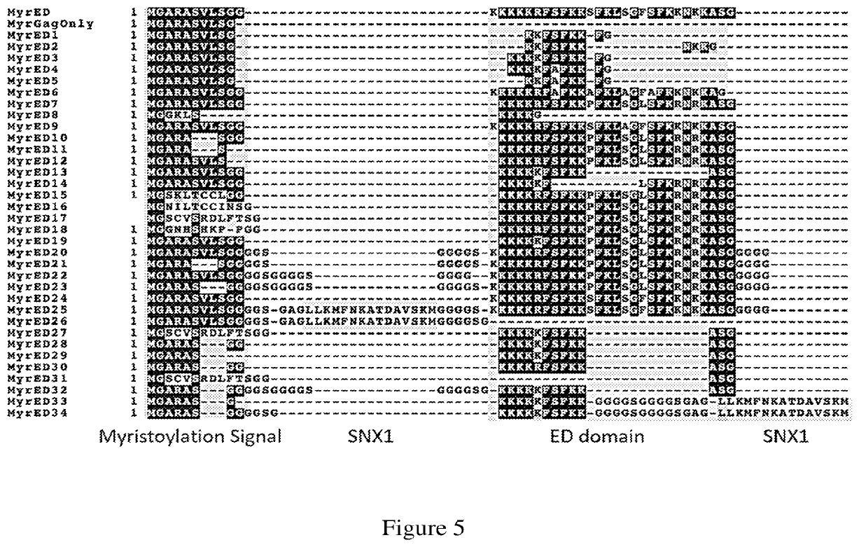

Thirty-four (34) variants of MyrED protein were designed to improve the vesicle targeting ability for the fusion protein. FIG. 5 shows the alignment of various MyrED sequences designed for this invention.

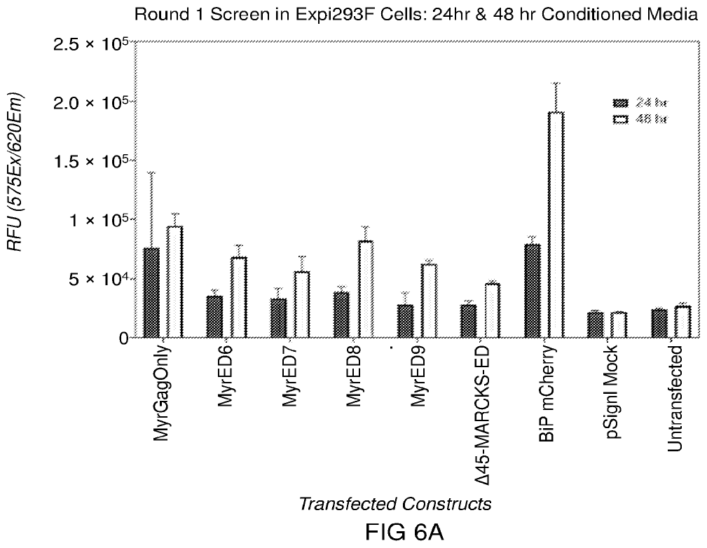

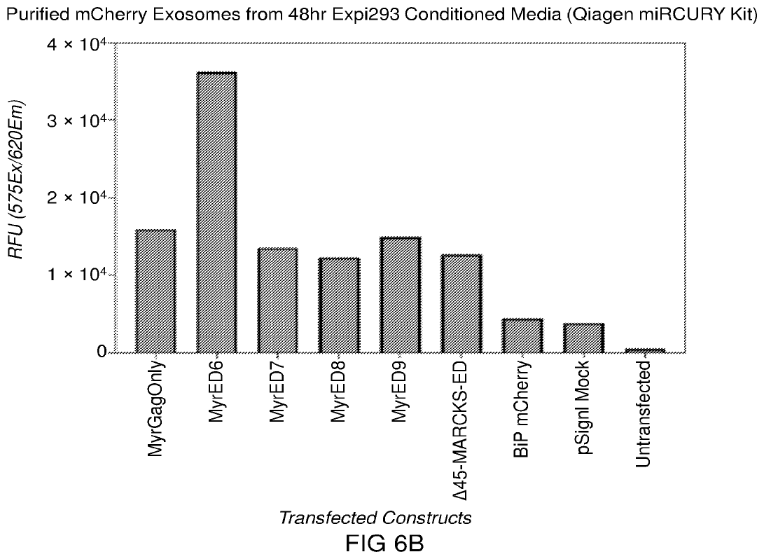

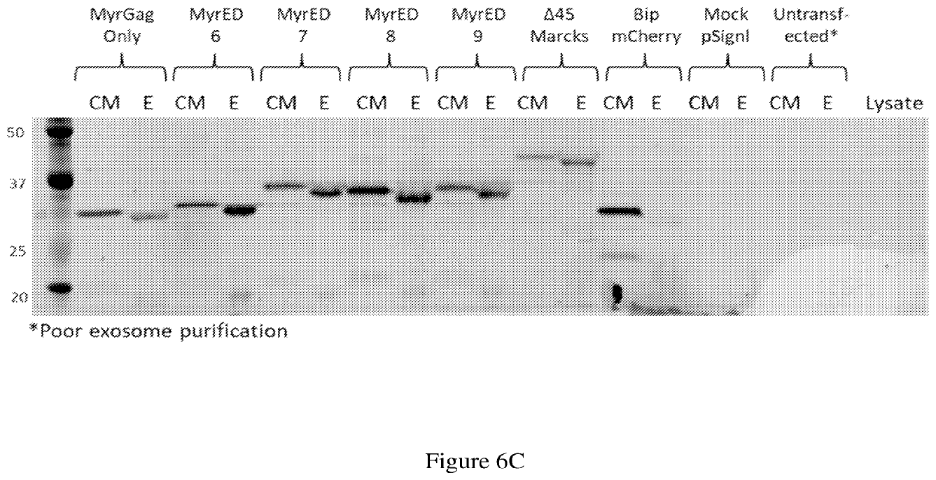

Expi293F cells were transfected with a polynucleotide encoding each of the MyrED protein variants, MyrGagOnly, MyrED6 (SEQ ID NO: 36), MyrED7 (SEQ ID NO: 37), MyrED8 (SEQ ID NO: 38), MyrED9 (SEQ ID NO: 39), and 445-MARCKS-ED, fused with mCherry. For negative control, (1) Expi293F cells transfected with Mock p-signal, and (2) untransfected Expi293F cells were used. The cells were allowed to grow in Expi293 expression medium. The fluorescence intensity of the conditioned media was measured at 24 hours and 48 hours as shown in FIG. 6A. The exosomes containing mCherry were purified from the conditioned media after 48 hours and relative fluorescence intensity was measured as shown in FIG. 6B. FIG. 6C shows western blot analysis showing that mCherry was targeted to exosomes in all MyrED variants tested for this experiment.

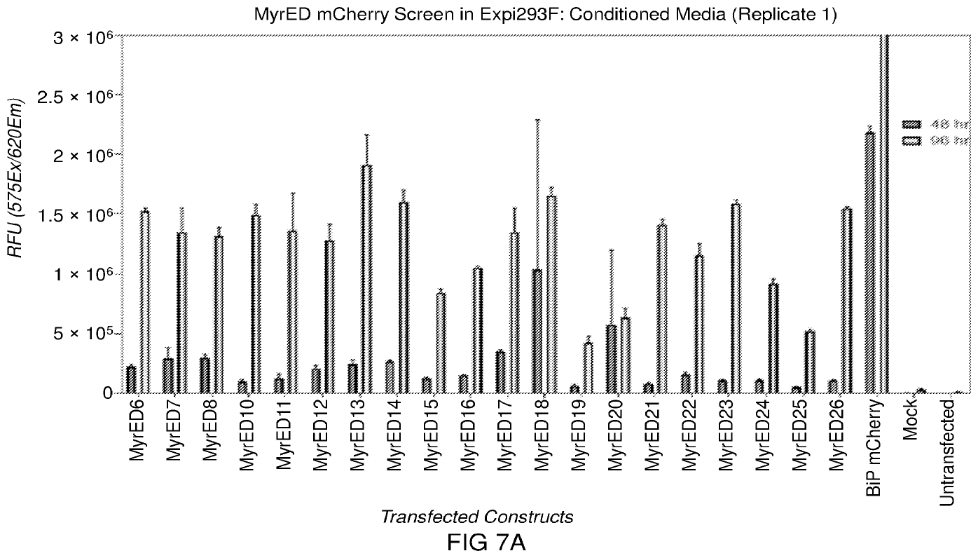

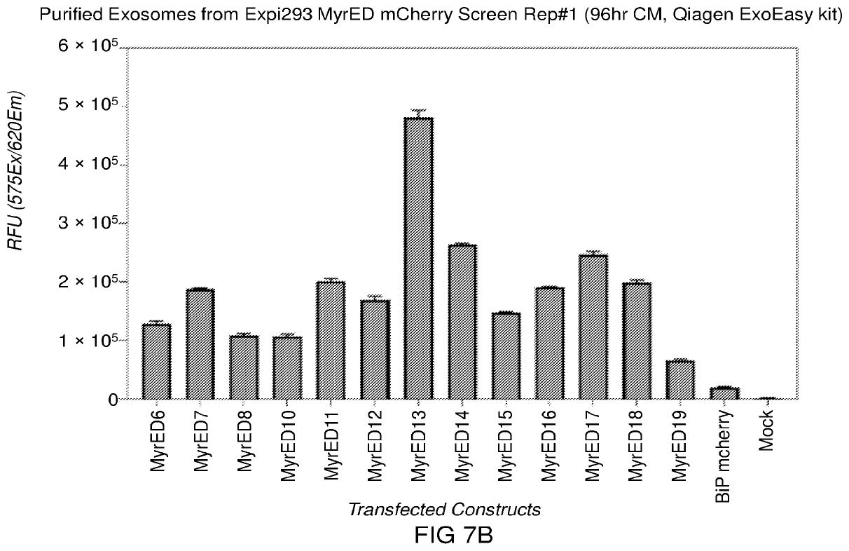

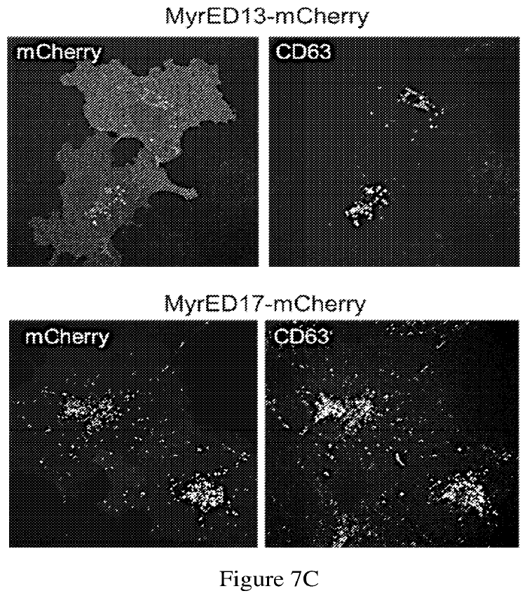

Similarly, Expi293F cells were transfected with a polynucleotide encoding each of the MyrED protein variants, MyrED6 (SEQ ID NO: 36), MyrED7 (SEQ ID NO: 37), MyrED8 (SEQ ID NO: 38), MyrED10 (SEQ ID NO: 40), MyrED11 (SEQ ID NO: 41), MyrED12 (SEQ ID NO: 42), MyrED13 (SEQ ID NO: 43), MyrED14 (SEQ ID NO: 44), MyrED15 (SEQ ID NO: 45), MyrED16 (SEQ ID NO: 46), MyrED17 (SEQ ID NO: 47), MyrED18 (SEQ ID NO: 48), MyrED19 (SEQ ID NO: 49), MyrED20 (SEQ ID NO: 50), MyrED21 (SEQ ID NO: 51), MyrED22 (SEQ ID NO: 52), MyrED23 (SEQ ID NO: 53), MyrED24 (SEQ ID NO: 54), MyrED25 (SEQ ID NO: 44), and MyrED26 (SEQ ID NO: 56), fused with mCherry. Expi293F cells were transfected with BiPa and mCherry as a secretory positive control. For negative control, (1) Expi293F cells transfected with Mock p-signal, and (2) untransfected Expi293F cells were used. The cells were allowed to grow in Expi293 expression medium. The fluorescence intensity of the conditioned media was measured at 48 hours and 96 hours as shown in FIG. 7A. The exosomes containing mCherry were purified from the Expi293 expression medium and relative fluorescence intensity was measured as shown in FIG. 7B. MyrED13 and MyrED17 with mCherry were further used to transfect Cos-7 cells. FIG. 7C shows mCherry (left) co-localizing with CD63 (right) for MyrED13 and MyrED17.

### Example-3

### [A] Determination of α-D-Glucopyranoside Protein Targeting Exosomes.

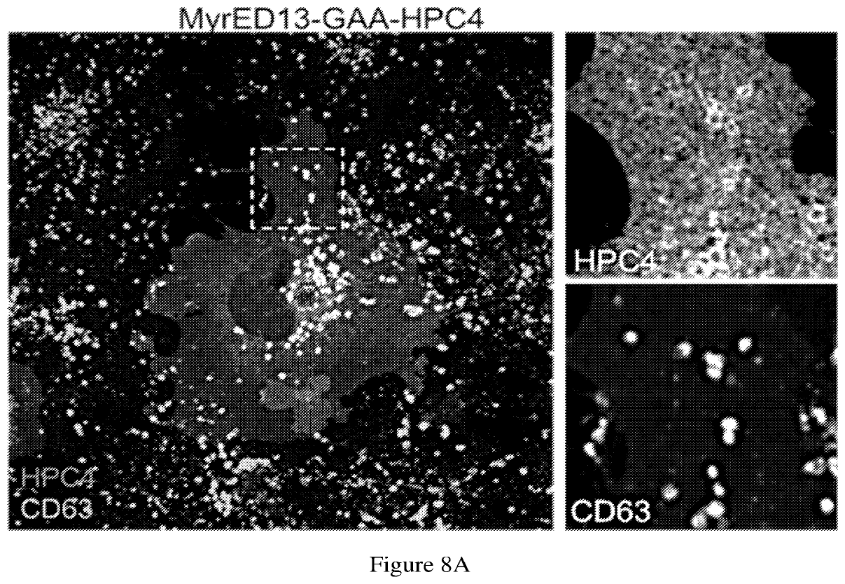

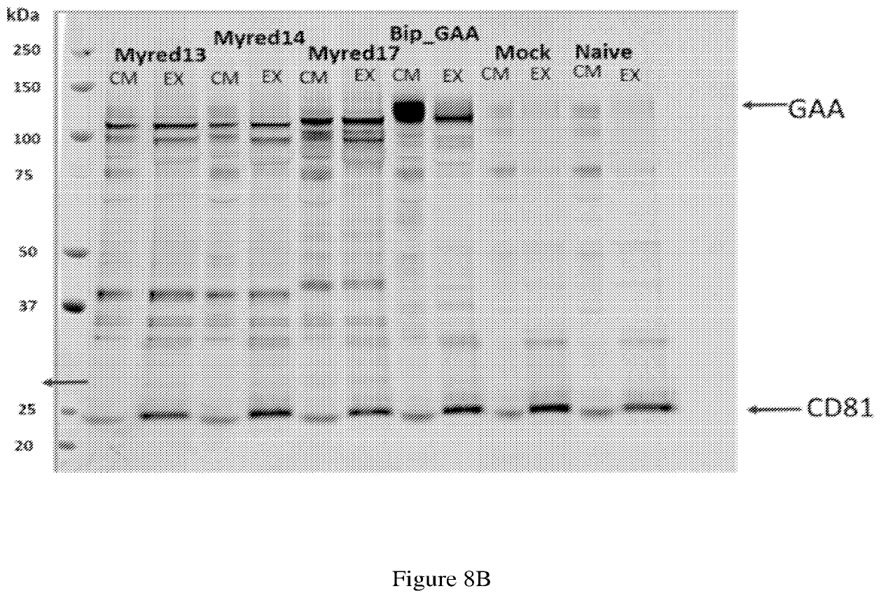

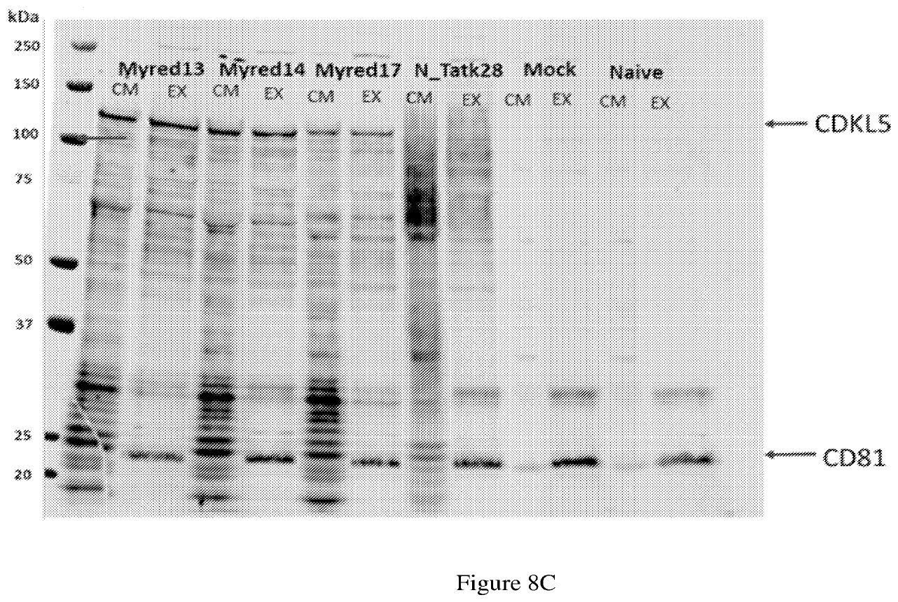

A construct encoding the fusion protein, MyrED13-GAA-HPC4, was used to transfect Cos-7 cells. CD63 protein is targeted in exosomes. FIG. 8A shows immuno-visualized image for Anti-HPC4 (upper right) and Anti-CD63 antibody directed against exosomal protein CD63 (lower right). The figure shows co-localization of the fusion protein with exosomal protein CD63.

Cells were transfected with constructs encoding GAA protein fused with MyrED13 (SEQ ID NO: 43), MyrED14 (SEQ ID NO: 44), and MyrED17 (SEQ ID NO: 47). Cells were transfected with BiP-GAA as a secretory positive control. For negative control, (1) cells transfected with Mock p-signal, and (2) untransfected cells were used. The cells were allowed to grow in expression medium. The exosomes containing the fusion protein were purified from the fluorescence conditioned media and estern blot analysis was performed. FIG. 8B shows that the fusion protein was targeted to the exosomes.

### [B] Determination of CDKL5 Protein Targeting Exosomes.

Cells were transfected with constructs encoding CDKL5 protein fusion with MyrED13 (SEQ ID NO: 43), MyrED14 (SEQ ID NO: 44), and MyrED17 (SEQ ID NO: 47). Cells were transfected with N_Tatk28 as a secretory positive control. For negative control, (1) cells transfected with Mock p-signal, and (2) untransfected cells were used. The cells were allowed to grow expression medium. The exosomes containing the fusion protein were purified from the fluorescence conditioned media and western blot analysis was performed. FIG. 7C shows that the fusion protein was targeted to the exosomes.

### Example-4

### [A] Determining Transduction Efficiency of Exosome In Vivo.

A construct for transduction encoding the fusion protein were prepared, wherein the fusion protein comprised MyrED27-mCherry (SEQ ID NO: 160), MyrED77-mCherry (MyrED77 shown as SEQ ID NO: 107), MyrED81-mCherry (SEQ ID NO: 161) or MyrED88-mCherry (SEQ ID NO: 162). Bip-mCherry was used as a control. Furthermore, each construct further comprised IRES-GFP marker. The marker functioned to identify transduced cells.

Each construct was used to transduce mouse embryos. Specifically, the embryo brains inside the uterus were directly injected with plasmids and/or electroporated enabling the plasmids to enter cells. The embryos were then harvested four days later at E18.5.

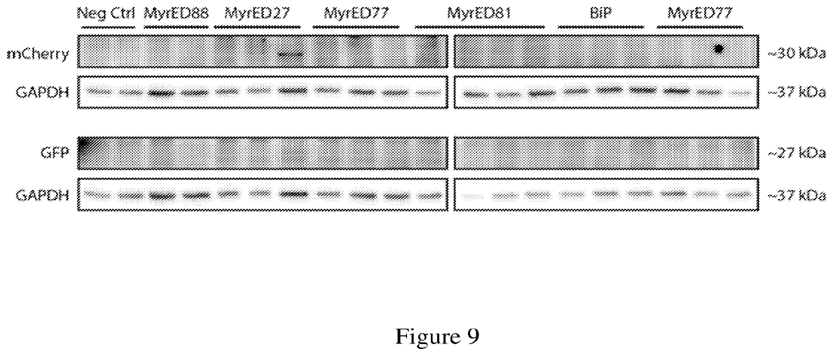

Liver extracts were analyzed for presence of mCherry protein in a western blot. FIG. 9 shows western blot analysis of mCherry and GFP expression in liver. The analysis suggests potential long-distance cross-correction and escape from blood-brain barrier.

Embryonic brain sections were fluorescently imaged for presence of GFP and mCherry. Only transfected cells expressed GFP, whereas cross-corrected cells only expressed mCherry. FIGS. 10A-10C shows an image analysis of MyrED27 mediated transduction of mCherry. FIG. 10A shows GFP transfected cells, FIG. 10B shows mCherry cross-corrected cells and FIG. 10C shows a merge image thereof. Combined analysis of FIGS. 9 and 10A-10C suggests that MyrED27 is an axonal targeting and membrane associated construct, which can be used for cross-correction. However, one or two embryos showed expression of mCherry in the liver.

FIGS. 11A-11C shows an image analysis of MyrED77 mediated transduction of mCherry. FIG. 11A shows GFP transfected cells, FIG. 11B shows mCherry cross-corrected cells and FIG. 11C shows a merge image thereof. Combined analysis of FIGS. 9 and 11A-11C suggests that MyrED77 has affinity towards axons and the construct can be used for cross-correction.

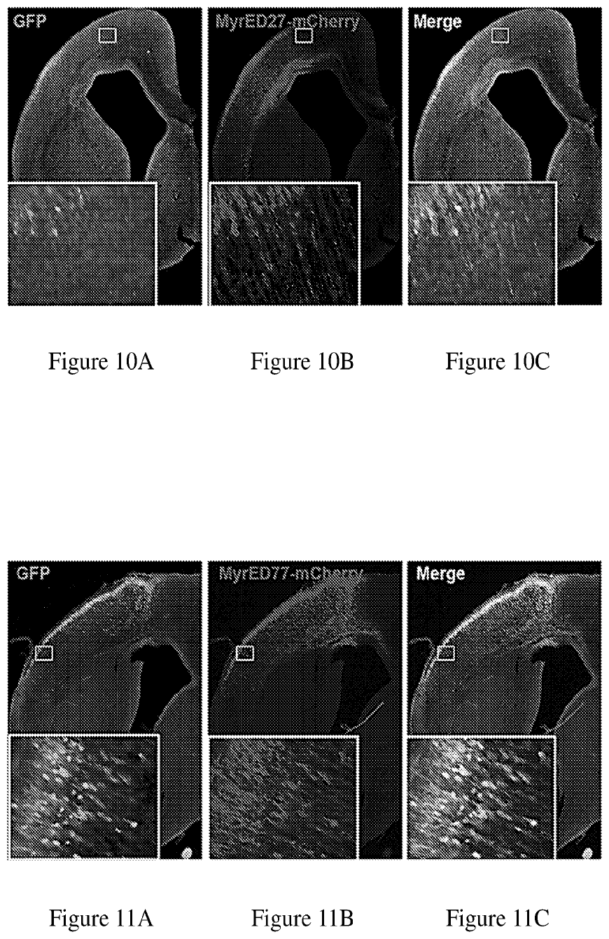

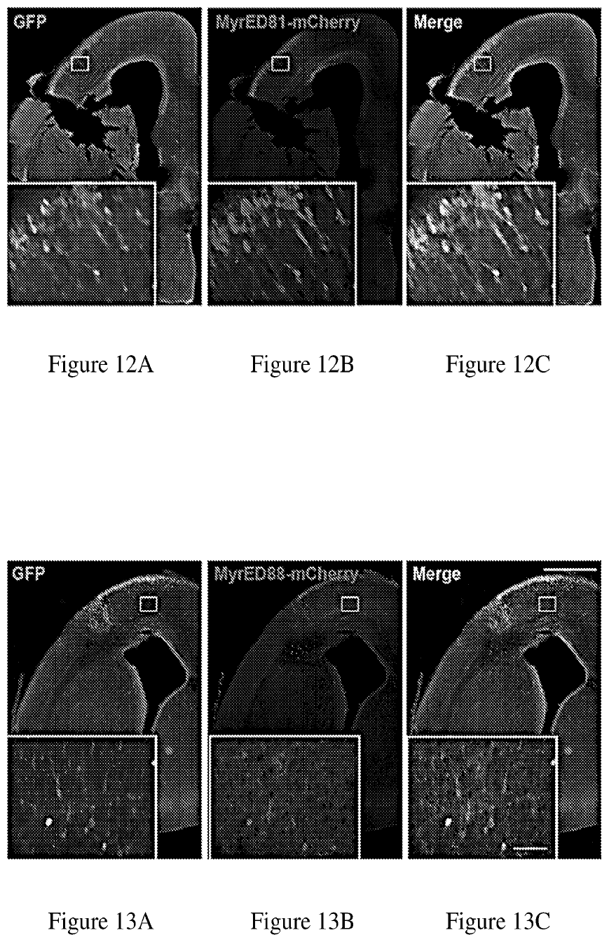

FIGS. 12A-12C shows an image analysis of MyrED81 mediated transduction of mCherry. FIG. 12A shows GFP transfected cells, FIG. 12B shows mCherry cross-corrected cells and FIG. 12C shows a merge image thereof. Combined analysis of FIGS. 9 and 12A-12C suggests that MyrED81 showed lower expression. However, the construct can be used for cross-correction.

FIGS. 13A-13C shows an image analysis of MyrED88 mediated transduction of mCherry. FIG. 13A shows GFP transfected cells, FIG. 13B shows mCherry cross-corrected cells and FIG. 13C shows a merge image thereof. Combined analysis of FIGS. 9 and 13A-13C suggests that MyrED88 is an outlier in the set. The construction can be used for cross-correction, but there is a diffusion signal throughout the entire parenchyma. High magnification reveals small red puncta, likely secreted extracellularly.

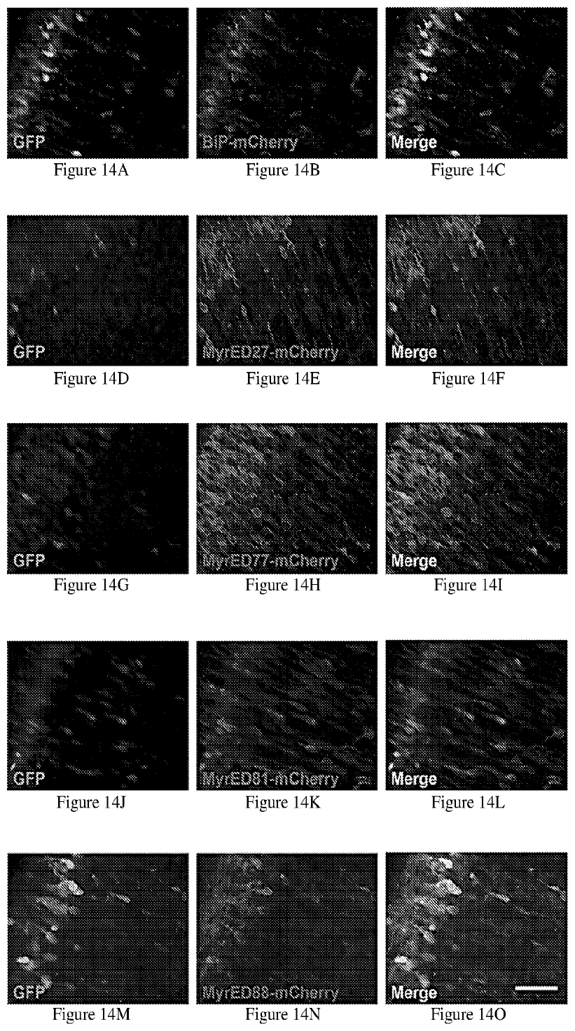

FIGS. 14A-140 shows comparative transduction efficiency of the fusion proteins, MyrED27-mCherry, MyrED77-mCherry, MyrED81-mCherry and MryED88-mCherry, against the positive control BiP-mCherry. FIGS. 14A, 14D, 14G, 14J and 14M show GFP transfected cells, FIGS. 14B, 14E, 14H, 14K and 14N show mCherry cross-corrected cells, and FIGS. 14C, 14F, 14I, 14L and 14O show merge images thereof. The image analysis also suggests that cross-correction is not evident with BiP-mCherry construct.

In conclusion, the MyrED27 construct is an axonal targeting and membrane associated construct. Presence of more red cells than green/yellow cells indicate MyrED27 mediated cross-correction. According to Western blot analysis, one or two embryos showed MyrED27 mediated mCherry expression in liver. The specificity of the technique was remarkable, such as, one brain hemisphere was transduced without affecting the other.

### Example-5

[A] CDKL5 Targeting with MyrED

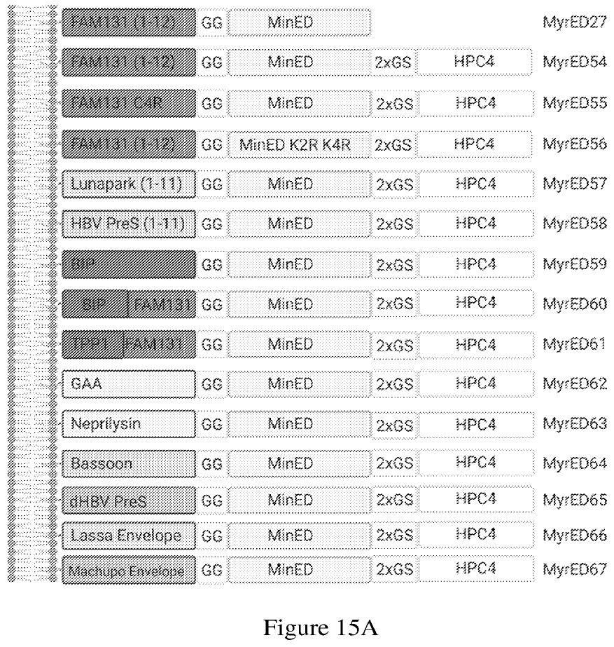

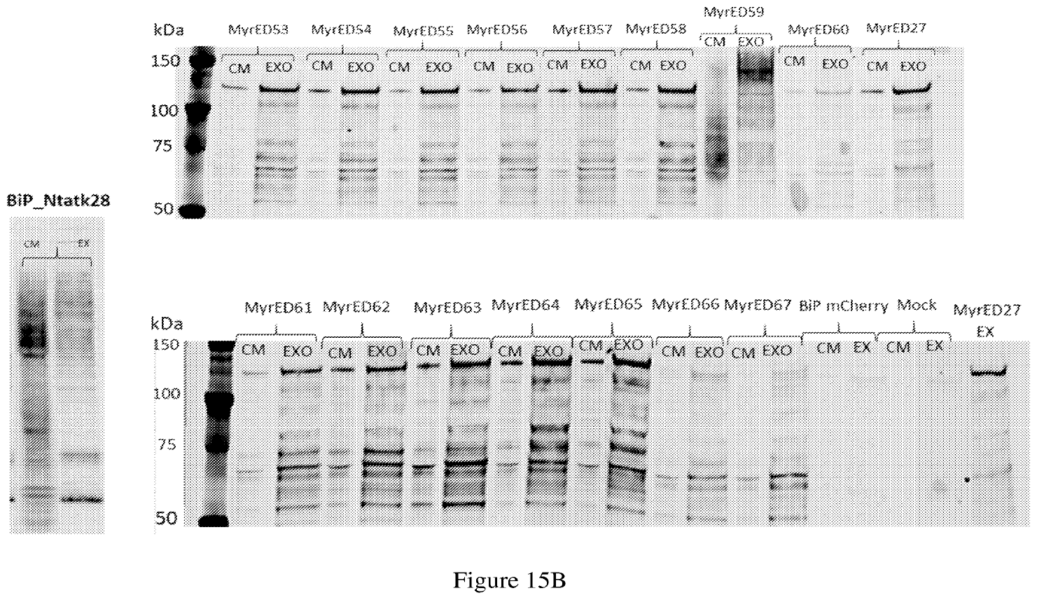

Cells were transfected with constructs encoding CDKL5 protein fusion with MyrED27 (SEQ ID NO: 57) and MyrED53-67 (SEQ ID NOS: 83-97). 9 μl of conditioned media (CM) was harvested 72 hours post transfection in Expi293F cells and 5 μg of exosomes (EXO) normalized based on total protein quantification were loaded on to respective lanes. Exosomes were enriched from 5 ml of conditioned media using Qiagen Exoeasy maxi kit. FIG. 15B is a western blot showing that the fusion protein was targeted to the exosomes.

### Example-6

[A] TPP1 Targeting with MyrED

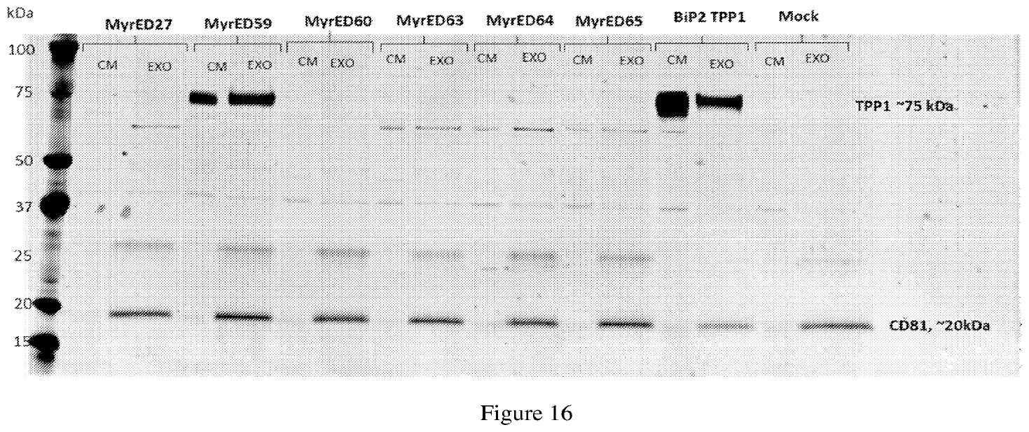

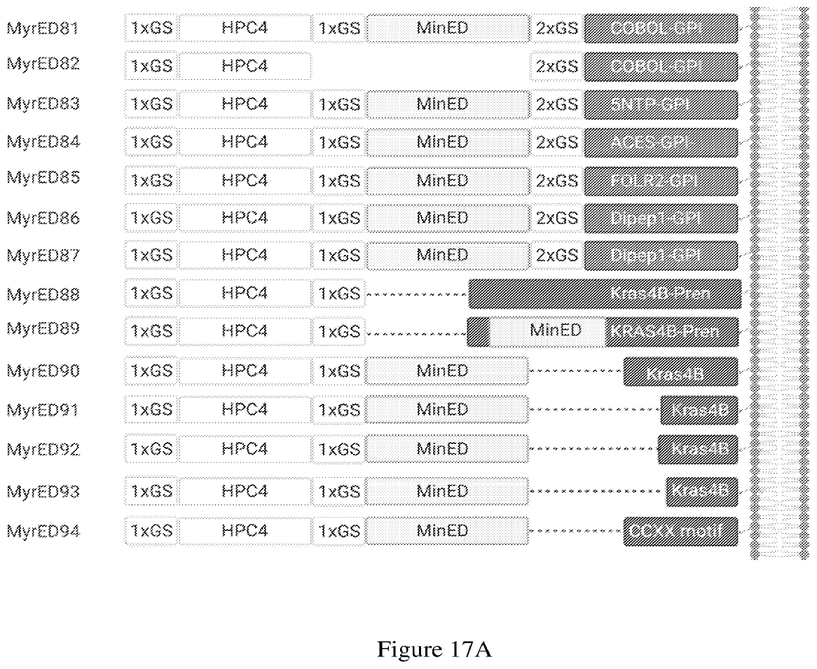

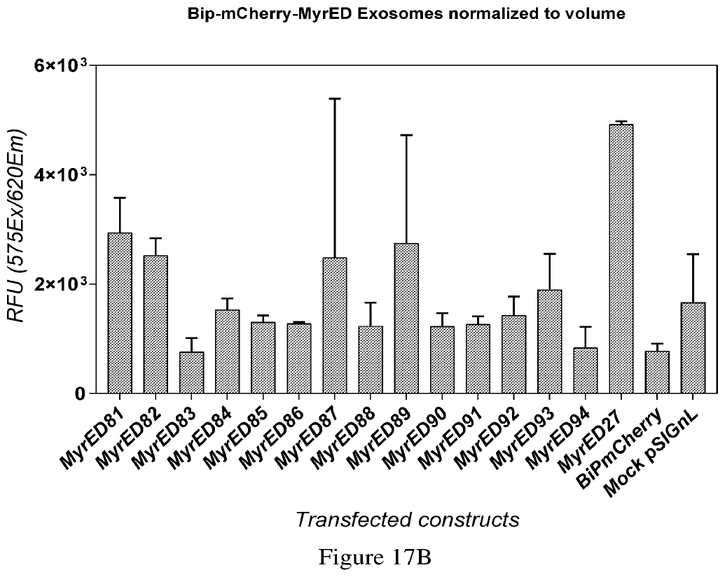

Cells were transfected with constructs encoding TPP1 protein fused with MyrED27 (SEQ ID NO: 57), MyrED 59 (SEQ ID NO: 89), MyrED60 (SEQ ID NO: 90), MyrED63 (SEQ ID NO: 93), MyrED64 (SEQ ID NO: 94) and MyrED65 (SEQ ID NO: 95). 9 μl of conditioned media (CM) harvested 72 hours post transfection in Expi293F cells and 4.5 μg of exosomes (EXO) normalized based on total protein quantification were loaded on to respective lanes. Exosomes were enriched from 5 ml of conditioned media using Qiagen Exoeasy maxi kit. FIG. 16 is a western blot showing that the fusion protein was targeted to the exosomes.

### Example-7

### [A] Testing of C-terminal Exosome Targeting Tags

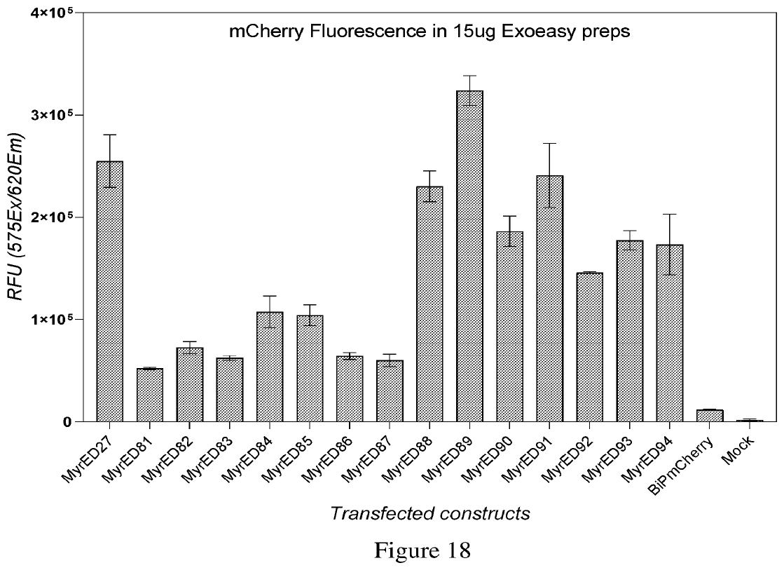

Cells were transfected with constructs encoding mCherry protein fused with MyrED27 (SEQ ID NO: 57) and MyrED81-94 (SEQ ID NOS: 111-124) and an mCherry fluorescence screen was performed using these various C terminally tagged exosome targeted variants. 15 μg of exosomes normalized based on total protein quantification were read at 575Ex/620 Em using Spectramax id5. Exosomes were enriched from 5 ml of conditioned media using Qiagen Exoeasy maxi kit. FIG. 18 shows that the fusion protein was targeted to the exosomes.

### Example-8

[A] GAA Targeting with MyrED

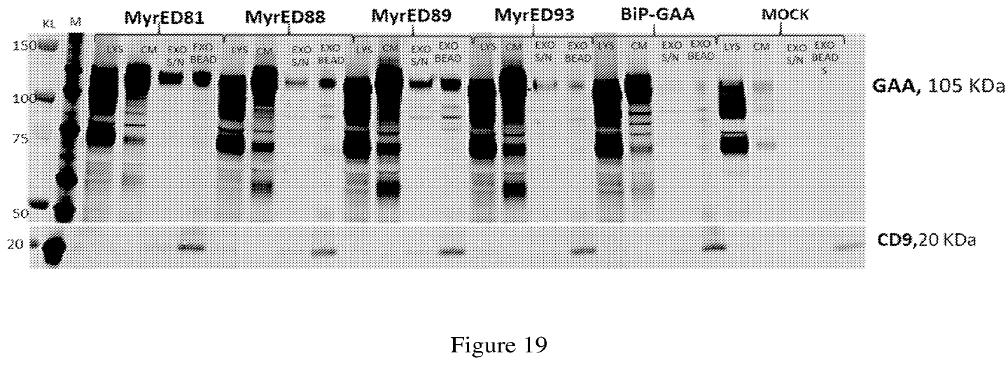

Cells were transfected with constructs encoding GAA protein fused with MyrED81 (SEQ ID NO: 111), MyrED88 (SEQ ID NO: 118), MyrED89 (SEQ ID NO: 119) and MyrED93 (SEQ ID NO: 123). BiP tagged GAA construct was regarded as negative control. 5 μg of total cell lysates (LYS) and 12 μl of conditioned media (CM) harvested 72 hours post transfection in Expi293F cells were loaded on to respective lanes. Exosomes were enriched from 2 ml of conditioned media using Miltenyi Biotec Exosome isolation kit pan, Human (MACS). 100 μl of isolated exosomes were spun down at 5000 g and both 12 μl of supernatant(EXO S/N) and 12 ul pelleted beads (EXO BEAD) were loaded on to respective lanes. FIG. 19 is a western blot showing GAA (˜105 kDa) and CD9 (˜20 kDa) Tetraspanin marker expression in these C-terminally tagged exosome targeted variants.

### Example-9

[A] NAGLU Targeting with MyrED

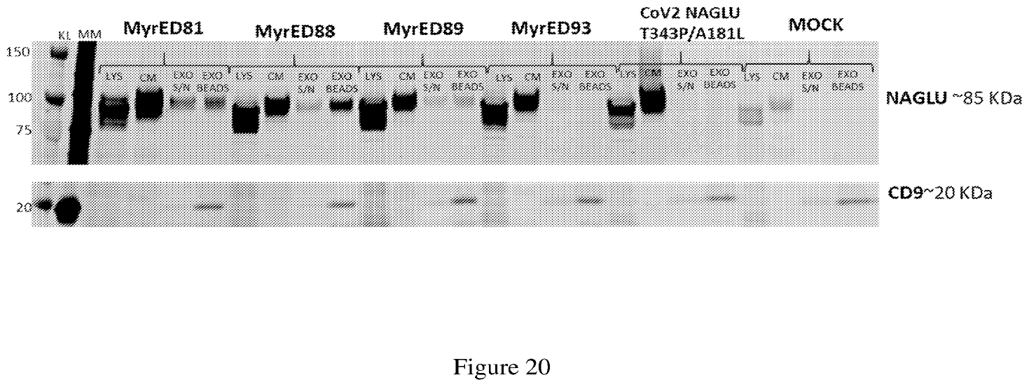

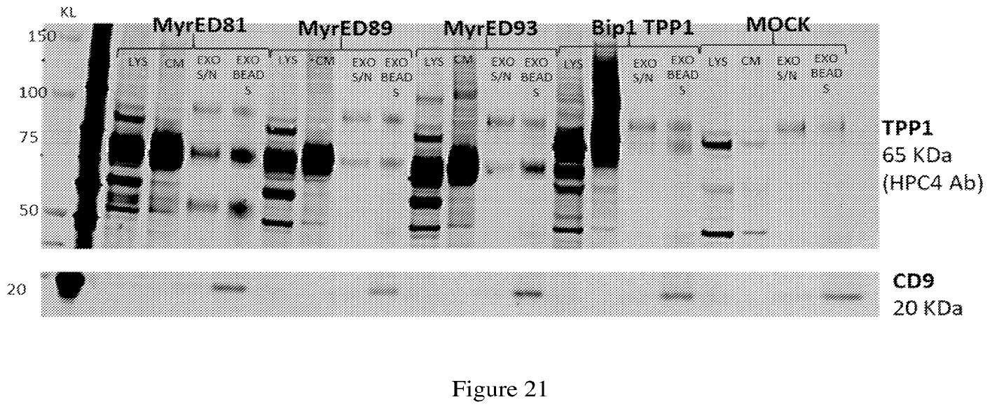

Cells were transfected with constructs encoding NAGLU protein fused with MyrED81 (SEQ ID NO: 111), MyrED88 (SEQ ID NO: 118), MyrED89 (SEQ ID NO: 119) and MyrED93 (SEQ ID NO: 123). COV2 NAGLU (T343P/A181L) was regarded as negative control. 5 μg of total cell lysates (LYS) and 12 μl of conditioned media (CM) harvested 72 hours post transfection in Expi293F cells were loaded on to respective lanes. Exosomes were enriched from 2 ml of conditioned media using Miltenyi Biotec Exosome isolation kit pan, Human (MACS). 100 μl of isolated exosomes were spun down at 5000 g and both 12 μl of supernatant(EXO S/N) and 12 μl pelleted beads (EXO BEAD) were loaded on to respective lanes. FIG. 20 is a western blot showing NAGLU (˜85 kDa) and CD9 (˜20 kDa) Tetraspanin marker expression in these C-terminally tagged exosome targeted variants.

### Example-10

[A] TPP1Targeting with MyrED

Cells were transfected with constructs encoding TPP1 protein fused with MyrED81 (SEQ ID NO: 111), MyrED89 (SEQ ID NO: 119) and MyrED93 (SEQ ID NO: 123). BiP1 tagged TPP1 construct was regarded as negative control. 5 μg of total cell lysates (LYS) and 12 μl of conditioned media (CM) harvested 72 hours post transfection in Expi293F cells were loaded on to respective lanes. Exosomes were enriched from 2 ml of conditioned media using Miltenyi Biotec Exosome isolation kit pan, Human (MACS). 100 μl of isolated exosomes were spun down at 5000 g and both 12 μl of supernatant (EXO S/N) and 12 μl pelleted beads (EXO BEAD) were loaded on to respective lanes. FIG. 20 is a western blot showing TPP1 (HPC4 Ab-˜65 kDa) and CD9 (˜20 kDa) Tetraspanin marker expression in these C-terminally tagged exosome targeted variants.

SEQUENCE LISTING

SEQ ID NO: 1 vMSP_001
MGARASVLSGG
SEQ ID NO: 2 vMSP_002
MGARASVLSG
SEQ ID NO: 3 vMSP_003
MGGKLS
SEQ ID NO: 4 vMSP_004
MGARASGG
SEQ ID NO: 5 vMSP_005
MGARAS
SEQ ID NO: 6 vMSP_006
MGARASVLS
SEQ ID NO: 7 vMSP_007
MGSKLTCCLGG
SEQ ID NO: 8 vMSP_008
MGNILTCCINS
SEQ ID NO: 9 vMSP_009
MGSCVSRDLFT
SEQ ID NO: 10 vMSP_0010
MGGNHSHKPP
SEQ ID NO: 11 vMSP_011
MGSCVSRDLFTS
SEQ ID NO: 12 vMSP_012
MGARASG
SEQ ID NO: 13 VED_001
KKKKKRFSFKKSFKLSGFSFKKNKKAS
SEQ ID NO: 14 VED_002
KKFSFKKFG
SEQ ID NO: 15 VED_003
KKFSFKKNKKG
SEQ ID NO: 16 VED_004
KKKKFSFKKFG
SEQ ID NO: 17 VED_005
KKKKFAFKKFG
SEQ ID NO: 18 VED_006
KKFAFKKFG
SEQ ID NO: 19 vED_007
KKKKKRFAFKKAFKLAGFAFKKNKKAG
SEQ ID NO: 20 VED_008
KKKKRFSFKKPFKLSGLSFKRNRKAS
SEQ ID NO: 21 VED_009
KKKKG
SEQ ID NO: 22 VED_010
KKKKKRFSFKKSFKLAGFSFKKNKKAS
SEQ ID NO: 23 VED_011
KKKKRFSFKKPFKLSGLSFKRNRKASG
SEQ ID NO: 24 VED_012
KKKKKFSFKKAS
SEQ ID NO: 25 VED_013
KKKKKFLSFKRNRKAS
SEQ ID NO: 26 VED_014
KKKKRFSFKKPFKLSGLSFKRNRKASGGGGG
SEQ ID NO: 27 VED_015
KKKKKFSFKKPFKLSGLSFKRNRKAS
SEQ ID NO: 28 VED_016
KKKKKRFSFKKPFKLSGLSFKRNRKAS
SEQ ID NO: 29 VED_017
KKKKRFSFKKAS
SEQ ID NO: 30 VED_018
KKKKKFSFKKGGGGSGGGGSGAGL
SEQ ID NO: 31 vMYRED_001
MGARASVLSGKKFSFKKFG
SEQ ID NO: 32 vMYRED_002
MGARASVLSGKKFSFKKNKKG
SEQ ID NO: 33 vMYRED_003
MGARASVLSGKKKKFSFKKFG
SEQ ID NO: 34 vMYRED_004
MGARASVLSGKKKKFAFKKFG
SEQ ID NO: 35 vMYRED_005
MGARASVLSGKKFAFKKFG
SEQ ID NO: 36 vMYRED_006
MGARASVLSGGKKKKKRFAFKKAFKLAGFAFKKNKKAG
SEQ ID NO: 37 vMYRED_007
MGARASVLSGGKKKKRFSFKKPFKLSGLSFKRNRKASG
SEQ ID NO: 38 vMYRED_008
MGGKLSKKKKG
SEQ ID NO: 39 vMYRED_009
MGARASVLSGGKKKKKRFSFKKSFKLAGFSFKKNKKASG
SEQ ID NO: 40 vMYRED_010
MGARASGGKKKKRFSFKKPFKLSGLSFKRNRKASG
SEQ ID NO: 41 vMYRED_011
MGARASKKKKRFSFKKPFKLSGLSFKRNRKASG
SEQ ID NO: 42 vMYRED_012
MGARASVLSKKKKRFSFKKPFKLSGLSFKRNRKASG
SEQ ID NO: 43 vMYRED_013
MGARASVLSGGKKKKKFSFKKASG
SEQ ID NO: 44 vMYRED_014
MGARASVLSGGKKKKKFLSFKRNRKASG
SEQ ID NO: 45 vMYRED_015
MGSKLTCCLGGKKKKRFSFKKPFKLSGLSFKRNRKASG
SEQ ID NO: 46 vMYRED_016
MGNILTCCINSGKKKKRFSFKKPFKLSGLSFKRNRKASG
SEQ ID NO: 47 vMYRED_017
MGSCVSRDLFTSGKKKKRFSFKKPFKLSGLSFKRNRKASG
SEQ ID NO: 48 vMYRED_018
MGGNHSHKPPGGKKKKRFSFKKPFKLSGLSFKRNRKASG
SEQ ID NO: 49 vMYRED_019
MGARASVLSGGKKKKKFSFKKPFKLSGLSFKRNRKASG
SEQ ID NO: 50 vMYRED_020
MGARASVLSGGGGSGGGGSKKKKKRFSFKKPFKLSGLSFKRNRKASGGGGG
SEQ ID NO: 51 vMYRED_021
MGARASGGGGSGGGGSKKKKKRFSFKKPFKLSGLSFKRNRKASGGGGG
SEQ ID NO: 52 vMYRED_022
MGARASVLSGGGGSGGGGSGGGGKKKKKRFSFKKPFKLSGLSFKRNRKASGGGGG
SEQ ID NO: 53 vMYRED_023
MGARASGGGGSGGGGSGGGGSKKKKKRFSFKKPFKLSGLSFKRNRKASGGGGG
SEQ ID NO: 54 vMYRED_024
MGARASVLSGGKKKKKRFSFKKSFKLSGFSFKKNKKASG
SEQ ID NO: 55 vMYRED_025
MGARASVLSGGGGSGAGLLKMFNKATDAVSKMGGGGSKKKKKRFSFKKSFKLSGFSFKKNKKASGGGGG
SEQ ID NO: 56 vMYRED_026
MGARASVLSGGGGSGAGLLKMFNKATDAVSKMGGGGSG
SEQ ID NO: 57 vMYRED_027
MGSCVSRDLFTSGGKKKKKFSFKKASG
SEQ ID NO: 58 vMYRED_028
MGARASGGKKKKKFSFKKASG
SEQ ID NO: 59 vMYRED_029
MGARASKKKKKFSFKKASG
SEQ ID NO: 60 vMYRED_030
MGARASGGKKKKRFSFKKASG
SEQ ID NO: 61 vMYRED_031
MGSCVSRDLFTSGGASG
SEQ ID NO: 62 vMYRED_032
MGARASGGGGSGGGGSGGGGSGKKKKKFSFKKASG
SEQ ID NO: 63 vMYRED_033
MGARASGKKKKKFSFKKGGGGSGGGGSGAGLLKMFNKATDAVSKM
SEQ ID NO: 64 vMYRED_034
MGARASGGGGSGKKKKKFSFKKGGGGSGGGGSGAGLLKMFNKATDAVSKM
SEQ ID NO: 65 vMYRED_035
MGARASVLSGGKKKKKFSFKKASGGGGRHRQPRGWEQLKSG
SEQ ID NO: 66 vMYRED_036
MGARASVLSGGKKKKKFSFKKASGGGGPRRARSVKSG
SEQ ID NO: 67 vMYRED_037
MGARASVLSGGKKKKKFSFKKASGGGGSRRKREVKSG
SEQ ID NO: 68 vMYRED_038
MGARASVLSGGKKKKKFSFKKASGGGKSRREVESG
SEQ ID NO: 69 vMYRED_039
MGARASVLSGGKKKKKFSFKKASGGGKKRKKRESG
SEQ ID NO: 70 vMYRED_040
MGARASVLSGGKKKKKFSFKKASGGGRARRESG
SEQ ID NO: 71 vMYRED_041
MGARASVLSGGKKKKKFSFKKRARRESG
SEQ ID NO: 72 vMYRED_042
MGARASVLSGGKKKKKFSFKKRKKRESG
SEQ ID NO: 73 vMYRED_043
MGSRVSRDLFTSGGASG
SEQ ID NO: 74 vMYRED_044
MGSAVSRDLFTSG
SEQ ID NO: 75 vMYRED_045
MGSCVSRDLFTSGGKKKKKFSFKKASGGGGRHRQPRGWEQLKSG
SEQ ID NO: 76 vMYRED_046
MGSCVSRDLFTSGGKKKKKFSFKKASGGGGPRRARSVKSG
SEQ ID NO: 77 vMYRED_047
MGSCVSRDLFTSGGKKKKKFSFKKASGGGGSRRKREVKSG
SEQ ID NO: 78 vMYRED_048
MGSCVSRDLFTSGGKKKKKFSFKKASGGGKSRREVESG
SEQ ID NO: 79 vMYRED_049
MGSCVSRDLFTSGGKKKKKFSFKKASGGGKKRKKRESG
SEQ ID NO: 80 vMYRED_050
MGSCVSRDLFTSGGKKKKKFSFKKASGGGRARRESG
SEQ ID NO: 81 vMYRED_051
MGSCVSRDLFTSGGKKKKKFSFKKRARRESG
SEQ ID NO: 82 vMYRED_052
MGSCVSRDLFTSGGKKKKKFSFKKRKKRESG
SEQ ID NO: 83 vMYRED_053
MGSCVSRDLFTSGGKKKKKFSFKKASGGGRARRESG
SEQ ID NO: 84 vMYRED_054
MGSCVSRDLFTSGGKKKKKFSFKKASGGGSGGGGSEDQVDPRLIDGKG
SEQ ID NO: 85 vMYRED_055
MGSRVSRDLFTSGGKKKKKFSFKKASGGGSGGGGSEDQVDPRLIDGKG
SEQ ID NO: 86 vMYRED_056
MGSCVSRDLFTSGGKRKRKFSFKRASGGGSGGGGSEDQVDPRLIDGKG
SEQ ID NO: 87 vMYRED_057
MGGLFSRWRTSGGKKKKKFSFKKASGGGSGGGGSEDQVDPRLIDGKG
SEQ ID NO: 88 vMYRED_058
MGQNLSTSNPLGGKKKKKFSFKKASGGGSGGGGSEDQVDPRLIDGKG
SEQ ID NO: 89 vMYRED_059
MKLSLVAAMLLLLSAARAGGKKKKKFSFKKASGGGSGGGGSEDQVDPRLIDGKG
SEQ ID NO: 90 vMYRED_060
MKLSLVAAMLLLLSAARAGSCVSRDLFTSGGKKKKKFSFKKASGGGSGGGGSEDQVDPRLIDGKG
SEQ ID NO: 91 vMYRED_061
MGLQACLLGLFALILSGKCGSCVSRDLFTSGGKKKKKFSFKKASGGGSGGGGSEDQVDPRLIDGKG
SEQ ID NO: 92 vMYRED_062
MGVRHPLCSRRLLAVCALVSLATAALLGGKKKKKFSFKKASGGGSGGGGSEDQVDPRLIDGKG
SEQ ID NO: 93 vMYRED_063
MGKSESQMDITDINTGGKKKKKFSFKKASGGGSGGGGSEDQVDPRLIDGKG
SEQ ID NO: 94 vMYRED_064
MGNEASLEGGAGEGPLPPGGSGGKKKKKFSFKKASGGGSGGGGSEDQVDPRLIDGKG
SEQ ID NO: 95 vMYRED_065
MGQQPAKSMDVRRIEGGELLLNQLAGRGGKKKKKFSFKKASGGGSGGGGSEDQVDPRLIDGKG
SEQ ID NO: 96 vMYRED_066
MGQIVTFFQEVPHVIEEVMNIVLIALSVLAVLKGLYNFATCGLVGLVTFLLLCGRSCTGGKKKKKFSFKKASGGGSGGG
GSEDQVDPRLIDGKG
SEQ ID NO: 97 vMYRED_067
MGQLISFFQEIPVFLQEALNIALVAVSLIAVIKGIINLYKSGLFQFIFFLLLAGRSCSGGKKKKKFSFKKASGGGSGGGGSE
DQVDPRLIDGKG
SEQ ID NO: 98 vMYRED_068
MGSCVSRDLFTSGGKKKKKFSFKKASGGGGRRARSVKSG
SEQ ID NO: 99 vMYRED_069
MGSCVSRDLFTSGGKKKKKFSFKKASGGGGPRRARSVSG
SEQ ID NO: 100 vMYRED_070
MGSCVSRDLFTSGGKKKKKFSFKKASGGGGPRRARSVKSEDQVDPRLIDGKG
SEQ ID NO: 101 vMYRED_071
MGNEASLEGGAGGGKKKKKFSFKKASGGGSGGGGSEDQVDPRLIDGKG
SEQ ID NO: 102 vMYRED_072
MGNEVSLEGGAGDGPLPPGGAGPGPGPGPGSHPQLPAAKKRKSLASRKPPVEDPAAEDLRTR
SEQ ID NO: 103 vMYRED_073
MGNEASLEGGAGEGPLPPGGSGGKKKKKFSFKKASGGGGPRRARSVKSGGGGSEDQVDPRLIDGKG
SEQ ID NO: 104 vMYRED_074
MGQQPAKSMDGGKKKKKFSFKKASGGGSGGGGSEDQVDPRLIDGKG
SEQ ID NO: 105 vMYRED_075
MKKKKKFSFKKASGGGSGGGGSEDQVDPRLIDGKG
SEQ ID NO: 106 vMYRED_076
MGCKWSKSSVIGWPAVRERMRRAEPAADGVGAVSRDLEKHGAITSSNTAANNAACAWLEAQEEEEVGFPGGGS
GGGGSEDQVDPRLIDGKG
SEQ ID NO: 107 vMYRED_077
MGCKWSKSSVIGWPAVRERMRRAEPAADGVGAVSRDLEKHGAITSSNTAANNAACAWLEAQEEEEVGFPGGGSK
KKKKFSFKKASGGGSGGGGSEDQVDPRLIDGKG
SEQ ID NO: 108 vMYRED_078
ASGGGGSEDQVDPRLIDGKSGGGKKKKKFSFKK
SEQ ID NO: 109 vMYRED_079
MKLSLVAAMLLLLSAARAGARASVLSGGGGKKKKKFSFKKASGGGSGGGGSEDQVDPRLIDGKG
SEQ ID NO: 110 vMYRED_080
MKLSLVAAMLLLLSAARAGNEASLEGGAGEGPLPPGGSGGGGKKKKKFSFKKASGGGSGGGGSEDQVDPRLIDGKG
SEQ ID NO: 111 vMYRED_081
ASGGGGSEDQVDPRLIDGKSGGGKKKKKFSFKKGGGGSGGGGAITCLSFITFYVAAFMVLL
SEQ ID NO: 112 vMYRED_082
ASGGGGSEDQVDPRLIDGKSGGGSGGGGAITCLSFITFYVAAFMVLL
SEQ ID NO: 113 vMYRED_083
ASGGGGSEDQVDPRLIDGKSGGGKKKKKFSFKKGGGGSGGGGARIKFSTGSHCHGSFSLIFLSLWAVIFVLYQ
SEQ ID NO: 114 vMYRED_084
ASGGGGSEDQVDPRLIDGKSGGGKKKKKFSFKKGGGGSGGGGKLLNATACDGELSSSGTSSSKGIIFYVLFSILYLIF
SEQ ID NO: 115 vMYRED_085
ASGGGGSEDQVDPRLIDGKSGGGKKKKKFSFKKGGGGSGGGGRRNVARYGRTVVVSGSHAAAVAPG
SEQ ID NO: 116 vMYRED_086
ASGGGGSEDQVDPRLIDGKSGGGKKKKKFSFKKGGGGSGGGGLGQLEASCRTNYGYSAAPSLHLPPGSLLASLVPLLL
LSLP
SEQ ID NO: 117 vMYRED_087
ASGGGGSEDQVDPRLIDGKSGGGKKKKKFSFKKGGGGSGGGGRTNYGYSAAPSLHLPPGSLLASLVPLLLLSLP
SEQ ID NO: 118 vMYRED_088
ASGGGGSEDQVDPRLIDGKSGGGKHKEKMSKDGKKKKKKSKTKCVIM
SEQ ID NO: 119 vMYRED_089
ASGGGGSEDQVDPRLIDGKSGGGKHKEKMSKDGKKKKKFSFKKSKTKCVIM
SEQ ID NO: 120 vMYRED_090
ASGGGGSEDQVDPRLIDGKSGGGKKKKKFSFKKSKTKCVIM
SEQ ID NO: 121 vMYRED_091
ASGGGGSEDQVDPRLIDGKSGGGKKKKKFSFKKCVIM
SEQ ID NO: 122 vMYRED_092
ASGGGGSEDQVDPRLIDGKSGGGKKKKKFSFKKRCVIM
SEQ ID NO: 123 vMYRED_093
ASGGGGSEDQVDPRLIDGKSGGGKKKKKFSFKKGGGSGGGGSCVIM
SEQ ID NO: 124 vMYRED_094
ASGGGGSEDQVDPRLIDGKSGGGKKKKKFSFKKGGGSGGGGSCCLL
SEQ ID NO: 125 vMYRED_095
MGNEASLEGGAGEGPLPPGGSGGKKKKKFSFKKASGGGKSRREVESGGGGDYKDDDDKG
SEQ ID NO: 126 vMYRED_096
MGNEASLEGGGGKKKKKFSFKKASGGGKSRREVESGGGGDYKDDDDKG
SEQ ID NO: 127 vMYRED_097
MGNEASLEGGGGKKKKKFSFKKASGGGG
SEQ ID NO: 128 vMYRED_098
MGQQPAKSMDVRRIEGGELLLNQLAGRGGKKKKKFSFKKASGGGKSRREVESGGGGDYKDDDDKG
SEQ ID NO: 129 vMYRED_099
MGQQPAKSMDVRRIEGGGKKKKKFSFKKASGGGKSRREVESGGGGDYKDDDDKG
SEQ ID NO: 130 vMYRED_100
MGQQPAKSMDVRRIEGGGKKKKKFSFKKASGGGG
SEQ ID NO: 131 vMYRED_101
MGNEASLEGGAGEGPLPPGGSGGKKKKKFSFKKASGGGKSRREVESGGGG
SEQ ID NO: 132 vMYRED_27b
MGSCVSRDLFTSGGKKKKKFSFKKASPRRARSVKSSGGGGDYKDDDDKG
SEQ ID NO: 133 vMYRED_27c
MGSCVSRDLFTSGGKKKKKFSFKKASGGGKSRREVESGGGGDYKDDDDKA
SEQ ID NO: 134 vMYRED_64b
MGNEASLEGGAGEGPLPPGGSGGKKKKKFSFKKASGGGGPRRARSVKSSGGGGDYKDDDDKA
SEQ ID NO: 135 vMYRED_64c
MGNEASLEGGAGEGPLPPGGSGGKKKKKFSFKKASGGGKSRREVESGGGGDYKDDDDKA
SEQ ID NO: 136 vMYRED_64d
MGNEASLEGGAGEGPLPPGGSGGKKKKKFSFKKASGGGKSRREVEA
SEQ ID NO: 137 vMYRED_65c
MGQQPAKSMDVRRIEGGELLLNQLAGRGGKKKKKFSFKKASGGGKSRREVESGGGGDYKDDDDKA
SEQ ID NO: 138 vMYRED_65d
MGQQPAKSMDVRRIEGGELLLNQLAGRGGKKKKKFSFKKASGGGKSRREVEA
SEQ ID NO: 139 vMYRED_81b
ASGGGGSDYKDDDDKRPRAVPTQSGGGSGGGGKKKKKFSFKKGGGGSGGGGAITCLSFITFYVAAFMVLL
SEQ ID NO: 140 vMYRED_88b
ASGGGGSDYKDDDDKRPRAVPTQSGGGSGGGGKHKEKMSKDGKKKKKKSKTKCVIM
SEQ ID NO: 141 vMYRED_89b
ASGGGGSDYKDDDDKRPRAVPTQSGGGSGGGGKHKEKMSKDGKKKKKFSFKKSKTKCVIM
SEQ ID NO: 142 vMYRED_93b
AGGGGSDYKDDDDKRPRAVPTQSGGGSGGGGKKKKKFSFKKGGGSGGGGSCVIM
SEQ ID NO: 143 vLINKER_01
G
SEQ ID NO: 144 vLINKER_02
SG
SEQ ID NO: 145 vLINKER_03
GG
SEQ ID NO: 146 vLINKER_04
GGSGGGGS
SEQ ID NO: 147 vLINKER_05
GGSGGGGSGGGG
SEQ ID NO: 148 vLINKER_06
GGSGGGGSGGGGS
SEQ ID NO: 149 vLINKER_07
GGSGAGLLKMFNKATDAVSKMGGGGS
SEQ ID NO: 150 vLINKER_08
GGSGAGLLKMFNKATDAVSKMGGGGSG
SEQ ID NO: 151 vLINKER_09
SSG
SEQ ID NO: 152 vLINKER_10
GGSGGGGSGGGGSG
SEQ ID NO: 153 vLINKER_11
GGSG
SEQ ID NO: 154 vSNX1_01
LLKMFNKATDAVSKM
SEQ ID NO: 155 vBIP_01
MKLSLVAAMLLLLSAARA
SEQ ID NO: 156 MBIP
MKLSLVAAMLLLLSLVAAMLLLLSAARA
SEQ ID NO: 157 MBIP2
MKLSLVAAMLLLLWVALLLLSAARA
SEQ ID NO: 158 MBIP3
MKLSLVAAMLLLLSLVALLLLSAARA
SEQ ID NO: 159 MBIP4
MKLSLVAAMLLLLALVALLLLSAARA
SEQ ID NO: 160 vMyrED27-mCherry
MGSCVSRDLFTSGGKKKKKFSFKKASGGGGGSGGGGGSAGSVSKGEEDNMAIIKEFMRFKVHMEGSVNGHEFEIE
GEGEGRPYEGTQTAKLKVTKGGPLPFAWDILSPQFMYGSKAYVKHPADIPDYLKLSFPEGFKWERVMNFEDGGVVT
VTQDSSLQDGEFIYKVKLRGTNFPSDGPVMQKKTMGWEASSERMYPEDGALKGEIKQRLKLKDGGHYDAEVKTTYK
AKKPVQLPGAYNVNIKLDITSHNEDYTIVEQYERAEGRHSTGGMDELYK
SEQ ID NO: 161 vMyrED81-mCherry
MKLSLVAAMLLLLSAARAVSKGEEDNMAIIKEFMRFKVHMEGSVNGHEFEIEGEGEGRPYEGTQTAKLKVTKGGPLP
FAWDILSPQFMYGSKAYVKHPADIPDYLKLSFPEGFKWERVMNFEDGGVVTVTQDSSLQDGEFIYKVKLRGTNFPSD
GPVMQKKTMGWEASSERMYPEDGALKGEIKQRLKLKDGGHYDAEVKTTYKAKKPVQLPGAYNVNIKLDITSHNED
YTIVEQYERAEGRHSTGGMDELYKGGGGSGGGGGSAASGGGGSEDQVDPRLIDGKSGGGKKKKKFSFKKGGGGSG
GGGAITCLSFITFYVAAFMVLL
SEQ ID NO: 162 v MyrED88-mCherry
MKLSLVAAMLLLLSAARAVSKGEEDNMAIIKEFMRFKVHMEGSVNGHEFEIEGEGEGRPYEGTQTAKLKVTKGGPLP
FAWDILSPQFMYGSKAYVKHPADIPDYLKLSFPEGFKWERVMNFEDGGVVTVTQDSSLODGEFIYKVKLRGTNFPSD
GPVMQKKTMGWEASSERMYPEDGALKGEIKQRLKLKDGGHYDAEVKTTYKAKKPVQLPGAYNVNIKLDITSHNED
YTIVEQYERAEGRHSTGGMDELYKGGGGSGGGGGSAASGGGGSEDQVDPRLIDGKSGGGKHKEKMSKDGKKKKK
KSKTKCVIM
SEQ ID NO: 163 vMyrEd64-mCherry
MGNEASLEGGAGEGPLPPGGSGGKKKKKFSFKKASGGGSGGGGSEDQVDPRLIDGKGGGGGSGGGGGSAGSVSK
GEEDNMAIIKEFMRFKVHMEGSVNGHEFEIEGEGEGRPYEGTQTAKLKVTKGGPLPFAWDILSPQFMYGSKAYVKH
PADIPDYLKLSFPEGFKWERVMNFEDGGVVTVTQDSSLQDGEFIYKVKLRGTNFPSDGPVMQKKTMGWEASSERM
YPEDGALKGEIKQRLKLKDGGHYDAEVKTTYKAKKPVQLPGAYNVNIKLDITSHNEDYTIVEQYERAEGRHSTGGMD
ELYK
SEQ ID NO: 164 v MyrED89-mcherry
MKLSLVAAMLLLLSAARAVSKGEEDNMAIIKEFMRFKVHMEGSVNGHEFEIEGEGEGRPYEGTQTAKLKVTKGGPLP
FAWDILSPQFMYGSKAYVKHPADIPDYLKLSFPEGFKWERVMNFEDGGVVTVTQDSSLODGEFIYKVKLRGTNFPSD
GPVMQKKTMGWEASSERMYPEDGALKGEIKQRLKLKDGGHYDAEVKTTYKAKKPVQLPGAYNVNIKLDITSHNED
YTIVEQYERAEGRHSTGGMDELYKGGGGSGGGGGSAASGGGGSEDQVDPRLIDGKSGGGKHKEKMSKDGKKKKK
FSFKKSKTKCVIM
SEQ ID NO: 165: KRAS4b
CVIM

1. A membrane associated-ED protein comprising a signal domain linked to an effector domain (ED) domain,
a. wherein the signal domain is directly linked to the effector domain; or
b. wherein the signal domain is connected to the effector domain by at least a first linker peptide, wherein the first linker peptide has an amino acid sequence comprising at least 70% sequence similarity SEQ ID NO: 143, SEQ ID NO: 144, SEQ ID NO: 145, SEQ ID NO: 146, SEQ ID NO: 147, SEQ ID NO: 148, SEQ ID NO: 149, SEQ ID NO: 150, SEQ ID NO: 151, SEQ ID NO: 152 or SEQ ID NO: 153.

2. The membrane associated-ED protein of claim 1, wherein the signal domain has an amino acid sequence comprising at least 70% sequence similarity to SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 155, SEQ ID NO: 156, SEQ ID NO: 157, SEQ ID NO: 158, SEQ ID NO: 159 or SEQ ID NO: 165, with the proviso that the resulting amino acid sequence of the membrane associated-ED protein is not SEQ ID NO: 38.

3. The membrane associated-ED protein of claim 1, wherein the signal domain has an amino acid sequence comprising at least 90% sequence similarity to SEQ ID NO:1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 155, SEQ ID NO: 156, SEQ ID NO: 157, SEQ ID NO: 158, SEQ ID NO: 159 or SEQ ID NO: 165, with the proviso that the resulting amino acid sequence of the membrane associated-ED protein is not SEQ ID NO: 38.

4. The membrane associated-ED protein of claim 1, wherein the signal domain has an amino acid sequence comprising SEQ ID NO:1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 155, SEQ ID NO: 156, SEQ ID NO: 157, SEQ ID NO: 158, SEQ ID NO: 159 or SEQ ID NO: 165, with the proviso that the resulting amino acid sequence of the membrane associated-ED protein is not SEQ ID NO: 38.

5. The membrane associated-ED protein of claim 1, wherein the ED domain has an amino acid sequence comprising at least 70% sequence similarity to SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29 or SEQ ID NO: 30, with the proviso that the resulting amino acid sequence of the membrane associated-ED protein is not SEQ ID NO: 38.

6. The membrane associated-ED protein of claim 1, wherein the ED domain has an amino acid sequence comprising at least 90% sequence similarity to SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29 or SEQ ID NO: 30, with the proviso that the resulting amino acid sequence of the membrane associated-ED protein is not SEQ ID NO: 38.

7. The membrane associated-ED protein of claim 1, wherein the ED domain has an amino acid sequence comprising SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29 or SEQ ID NO: 30, with the proviso that the resulting amino acid sequence of the membrane associated-ED protein is not SEQ ID NO: 38.

8. The membrane associated-ED protein of claim 1, wherein the membrane associated-ED protein has an amino acid sequence comprising at least 70% sequence similarity to SEQ ID NO:31, SEQ ID NO: 32, SEQ ID NO: 33, SEQ ID NO: 34, SEQ ID NO: 35, SEQ ID NO: 36, SEQ ID NO: 37, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 121, SEQ ID NO: 122, SEQ ID NO: 123, SEQ ID NO: 124, SEQ ID NO: 125, SEQ ID NO: 126, SEQ ID NO: 127, SEQ ID NO: 128, SEQ ID NO: 129, SEQ ID NO: 130, SEQ ID NO: 131, SEQ ID NO: 132, SEQ ID NO: 133, SEQ ID NO: 134, SEQ ID NO: 135, SEQ ID NO: 136, SEQ ID NO: 137, SEQ ID NO: 138, SEQ ID NO: 139, SEQ ID NO: 140, SEQ ID NO: 141 or SEQ ID NO: 142.

9. The membrane associated-ED protein of claim 1, wherein the membrane associated-ED protein has an amino acid sequence comprising at least 90% sequence similarity to SEQ ID NO:31, SEQ ID NO: 32, SEQ ID NO: 33, SEQ ID NO: 34, SEQ ID NO: 35, SEQ ID NO: 36, SEQ ID NO: 37, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 121, SEQ ID NO: 122, SEQ ID NO: 123, SEQ ID NO: 124, SEQ ID NO: 125, SEQ ID NO: 126, SEQ ID NO: 127, SEQ ID NO: 128, SEQ ID NO: 129, SEQ ID NO: 130, SEQ ID NO: 131, SEQ ID NO: 132, SEQ ID NO: 133, SEQ ID NO: 134, SEQ ID NO: 135, SEQ ID NO: 136, SEQ ID NO: 137, SEQ ID NO: 138, SEQ ID NO: 139, SEQ ID NO: 140, SEQ ID NO: 141 or SEQ ID NO: 142.

10. The membrane associated-ED protein of claim 1, wherein the membrane associated-ED protein has an amino acid sequence comprising SEQ ID NO:31, SEQ ID NO: 32, SEQ ID NO: 33, SEQ ID NO: 34, SEQ ID NO: 35, SEQ ID NO: 36, SEQ ID NO: 37, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 121, SEQ ID NO: 122, SEQ ID NO: 123, SEQ ID NO: 124, SEQ ID NO: 125, SEQ ID NO: 126, SEQ ID NO: 127, SEQ ID NO: 128, SEQ ID NO: 129, SEQ ID NO: 130, SEQ ID NO: 131, SEQ ID NO: 132, SEQ ID NO: 133, SEQ ID NO: 134, SEQ ID NO: 135, SEQ ID NO: 136, SEQ ID NO: 137, SEQ ID NO: 138, SEQ ID NO: 139, SEQ ID NO: 140, SEQ ID NO: 141 or SEQ ID NO: 142.

11. The membrane associated-ED protein of claim 1 further comprising a curvature sensing domain.

12. The membrane associated-ED protein of claim 1, wherein the N-terminus of membrane associated-ED protein does not have methionine.

13. A polynucleotide comprising a nucleotide sequence encoding the membrane associated-ED protein of claim 1.

14. A fusion protein comprising a vesicle targeting protein and a protein of interest, wherein the vesicle targeting protein comprises a lipid conjugating domain and an effector domain (ED); and
(a) wherein the vesicle targeting protein comprises the lipid conjugating domain directly linked to the effector domain (ED); or
(b) wherein the vesicle targeting protein comprises the lipid conjugating domain linked to the effector domain (ED) by a second linker peptide, wherein the second linker peptide has an amino acid sequence comprising at least 70% sequence similarity to SEQ ID NO: 143, SEQ ID NO: 144, SEQ ID NO: 145, SEQ ID NO: 146, SEQ ID NO: 147, SEQ ID NO: 148, SEQ ID NO: 149, SEQ ID NO: 150, SEQ ID NO: 151, SEQ ID NO: 152 or SEQ ID NO: 153.

15. The fusion protein of claim 14, wherein the vesicle targeting protein has an amino acid sequence comprising at least 70% sequence similarity to SEQ ID NO:31, SEQ ID NO: 32, SEQ ID NO: 33, SEQ ID NO: 34, SEQ ID NO: 35, SEQ ID NO: 36, SEQ ID NO: 37, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 121, SEQ ID NO: 122, SEQ ID NO: 123, SEQ ID NO: 124, SEQ ID NO: 125, SEQ ID NO: 126, SEQ ID NO: 127, SEQ ID NO: 128, SEQ ID NO: 129, SEQ ID NO: 130, SEQ ID NO: 131, SEQ ID NO: 132, SEQ ID NO: 133, SEQ ID NO: 134, SEQ ID NO: 135, SEQ ID NO: 136, SEQ ID NO: 137, SEQ ID NO: 138, SEQ ID NO: 139, SEQ ID NO: 140, SEQ ID NO: 141 or SEQ ID NO: 142.

16. The fusion protein of claim 14, wherein the vesicle targeting protein has an amino acid sequence comprising at least 90% sequence similarity to SEQ ID NO:31, SEQ ID NO: 32, SEQ ID NO: 33, SEQ ID NO: 34, SEQ ID NO: 35, SEQ ID NO: 36, SEQ ID NO: 37, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 121, SEQ ID NO: 122, SEQ ID NO: 123, SEQ ID NO: 124, SEQ ID NO: 125, SEQ ID NO: 126, SEQ ID NO: 127, SEQ ID NO: 128, SEQ ID NO: 129, SEQ ID NO: 130, SEQ ID NO: 131, SEQ ID NO: 132, SEQ ID NO: 133, SEQ ID NO: 134, SEQ ID NO: 135, SEQ ID NO: 136, SEQ ID NO: 137, SEQ ID NO: 138, SEQ ID NO: 139, SEQ ID NO: 140, SEQ ID NO: 141 or SEQ ID NO: 142.

17. The fusion protein of claim 14, wherein the vesicle targeting protein has an amino acid sequence comprising SEQ ID NO:31, SEQ ID NO: 32, SEQ ID NO: 33, SEQ ID NO: 34, SEQ ID NO: 35, SEQ ID NO: 36, SEQ ID NO: 37SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 121, SEQ ID NO: 122, SEQ ID NO: 123, SEQ ID NO: 124, SEQ ID NO: 125, SEQ ID NO: 126, SEQ ID NO: 127, SEQ ID NO: 128, SEQ ID NO: 129, SEQ ID NO: 130, SEQ ID NO: 131, SEQ ID NO: 132, SEQ ID NO: 133, SEQ ID NO: 134, SEQ ID NO: 135, SEQ ID NO: 136, SEQ ID NO: 137, SEQ ID NO: 138, SEQ ID NO: 139, SEQ ID NO: 140, SEQ ID NO: 141 or SEQ ID NO: 142.

18. The fusion protein of claim 14, wherein the lipid conjugating domain comprises one or more signal domains, the signal domain comprises one or more of a myristoylation signal domain, a palmitoylation signal domain, a prenylation signal domain a glycosylphosphatidylinsiton anchor protein and BiP secretion signal domain.

19. The fusion protein of claim 18, wherein the lipid conjugating domain is the myristoylation signal domain.

20. The vesicle targeting protein of claim 18, wherein the amino acid sequence of the signal domain has at least 70% similarity to the myristoylation signal domain of MARCKS, MARCKSL1 or BASP1 family of proteins.

21-94. (canceled)
